(19) United States
(12) Patent Application Publication
(10) Pub. No.: US 2025/0107438 A1
(43) Pub. Date: Mar. 27, 2025

(54) ORGANIC ELECTROLUMINESCENT COMPOUND, A PLURALITY OF HOST MATERIALS, AND ORGANIC ELECTROLUMINESCENT DEVICE COMPRISING THE SAME

(71) Applicant: DuPont Specialty Materials Korea Ltd., Chungcheongnam-do (KR)

(72) Inventors: Ji-Won UM, Gyeonggi-do (KR); Sang-Hee CHO, Gyeonggi-do (KR)

(21) Appl. No.: 18/782,191

(22) Filed: Jul. 24, 2024

(30) Foreign Application Priority Data
Sep. 22, 2023 (KR) 10-2023-0127033
Jul. 12, 2024 (KR) 10-2024-0092441

(51) Int. Cl. H10K 85/60; C07D 498/06; C09K 11/06; H10K 50/11; H10K 101/00

(52) U.S. Cl. CPC H10K 85/657; C07D 498/06; C09K 11/06; H10K 85/636; H10K 85/654; H10K 85/6574; C09K 2211/1018; H10K 50/11; H10K 2101/90

(57) ABSTRACT

The present disclosure relates to an organic electroluminescent compound, a plurality of host materials, and an organic electroluminescent device comprising the same. An organic electroluminescent device with improved luminous efficiency and/or lifespan properties can be provided by comprising compounds according to the present disclosure, or by comprising the specific combination of compounds according to the present disclosure as a plurality of host materials.

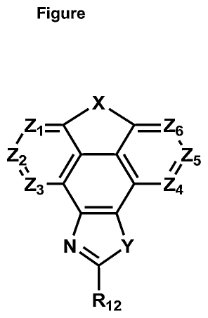

### TECHNICAL FIELD

The present disclosure relates to an organic electroluminescent compound, a plurality of host materials, and an organic electroluminescent device comprising the same.

### BACKGROUND ART

A small molecular green organic electroluminescent device (OLED) was first developed by Tang, et al., of Eastman Kodak in 1987, utilizing a TPD/ALq3 bi-layer consisting of a light-emitting layer and a charge transport layer. Thereafter, OLED development progressed rapidly, leading to commercialization. Currently, OLEDs primarily use phosphorescent materials with excellent luminous efficiency in panel implementation. However, in various applications such as TVs and lighting, the lifespan of OLEDs is often insufficient, and higher efficiency of OLEDs is still required. Generally, the lifespan of an OLED decreases as its luminance increases. Thus, OLEDs with high luminous efficiency and/or extended lifespan are essential for long-term use and high-resolution displays.

In order to improve luminous efficiency, driving voltage, and/or lifespan, various materials or concepts for an organic layer of an organic electroluminescent device have been proposed, but these did not prove satisfactory in practical use. In addition, there is a continuous demand for the development of light-emitting materials with enhanced performance, such as improved driving voltage, luminous efficiency, power efficiency, and/or lifetime properties, as compared to combinations of previously disclosed specific compounds.

In contrast, Korean Patent Application Laid-Open No. 10-2020-0013600 discloses phenanthrooxazole derivatives as compounds comprised in a plurality of host materials. Nevertheless, the aforementioned reference fails to specifically disclose a compound with a particular structure and a plurality of host materials comprising the same as claimed in the present disclosure. In addition, there is a continuous demand for the development of light-emitting materials with enhanced performance, such as improved driving voltage, luminous efficiency, and/or lifetime properties, as compared to combinations of previously disclosed specific compounds.

### DISCLOSURE OF INVENTION

### Technical Problem

The objective of the present disclosure is to provide an organic electroluminescent compound with a new structure suitable for application to an organic electroluminescent device. Another objective of the present disclosure is to provide an improved plurality of host materials capable of providing an organic electroluminescent device having improved luminous efficiency and/or lifetime properties. Further still, another objective of the present disclosure is to provide an organic electroluminescent device with improved luminous efficiency and/or lifetime properties by comprising a compound or a specific combination of compounds of the present disclosure.

### Solution to Problem

As a result of intensive study to solve the technical problems, the present inventors found that the above objective can be achieved by an organic electroluminescent compound represented by the following formula 1; or a plurality of host materials comprising at least one first host material comprising an organic electroluminescent compound according to the following formula 1, and at least one second host material different from the first host material.

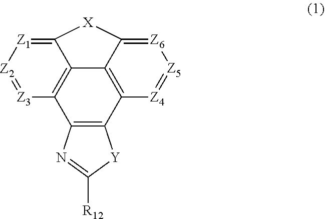

In formula 1,

X represents NR₁, O, S, Se, CR₂R₃, or SiR₄R₅;

R₁ to R₅ each independently represent a substituted or unsubstituted (C1-C30)alkyl, a substituted or unsubstituted (C6-C30)aryl, or a substituted or unsubstituted (3- to 30-membered)heteroaryl;

Y represents O, S, Se, or Te;

Z₁ to Z₆ each independently represent N or CR₁₁;

R₁₁ represents hydrogen, deuterium, or -L₁—R₁₃;

R₁₂ represents -L₂—R₁₄;

L₁ and L₂ each independently represent a single bond, a substituted or unsubstituted (C6-C30)arylene, or a substituted or unsubstituted (3- to 30-membered)heteroarylene;

R₁₃ and R₁₄ each independently represent a substituted or unsubstituted (C1-C30)alkyl, a substituted or unsubstituted (C6-C30)aryl, a substituted or unsubstituted (3- to 30-membered) heteroaryl, a substituted or unsubstituted (C3-C30)cycloalkyl, -L′₁—SiR′₁R′₂R′₃, -L′₂—GeR′₄R′₅R′₆, -L′₃—NR′₇R′₈, -L′₄(NR′₉R′₁₀)(NR′₁₁R′₁₂), -L′₅—NR′₁₃R′₁₄, or -L′₆—(NR′₁₅)—L′₇(NR′₁₆R′₁₇);

L′₁ to L′₇ each independently represent a single bond, a substituted or unsubstituted (C6-C30)arylene, or a substituted or unsubstituted (3- to 30-membered)heteroarylene; and

R′₁ to R′₁₇ each independently represent a substituted or unsubstituted (C1-C30)alkyl, a substituted or unsubstituted (C6-C30)aryl, a substituted or unsubstituted (3- to 30-membered) heteroaryl, or a substituted or unsubstituted (C3-C30)cycloalkyl.

### Advantageous Effects of Invention

The organic electroluminescent compound according to the present disclosure exhibits performances suitable for using it in an organic electroluminescent device. In addition, an organic electroluminescent device having improved driving voltage, luminous efficiency and/or lifetime properties compared to conventional organic electroluminescent devices is provided by comprising the compound according to the present disclosure as a single host material, or a plurality of compounds as host materials, and it is possible to produce a display system or a lighting system using the same.

### BRIEF DESCRIPTION OF THE FIGURE

The Figure illustrates representative formula for the organic electroluminescent compound according to the present disclosure.

### MODE FOR THE INVENTION

Hereinafter, the present disclosure will be described in detail. However, the following description is intended to explain the present disclosure and is not meant in any way to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of host materials” in the present disclosure means a host material comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, the plurality of host materials of the present disclosure is a combination of at least two host materials, and may selectively further comprise conventional materials comprised in an organic electroluminescent material. At least two compounds comprised in the plurality of host materials of the present disclosure may be comprised together in one light-emitting layer or may each be comprised in different light-emitting layers. For example, the at least two host materials may be mixture-evaporated or co-evaporated, or may be individually evaporated.

The term “a hole transport zone” in the present disclosure means a region where holes move between the first electrode and the light-emitting layer, and may include, for example, at least one of a hole injection layer, a hole transport layer, a hole auxiliary layer, a light-emitting auxiliary layer, and an electron blocking layer. The hole injection layer, the hole transport layer, the hole auxiliary layer, the light-emitting auxiliary layer, and the electron blocking layer may each be a single layer or a plurality of layers in which two or more layers or three or more layers are stacked. According to one embodiment of the present disclosure, the hole transport zone may include a first hole transport layer and a second hole transport layer, and may additionally include a third hole transport layer. The second hole transport layer and the third hole transport layer may be at least one of a plurality of hole transport layers and may include at least one of a hole auxiliary layer, a light-emitting auxiliary layer, and an electron blocking layer. In addition, according to another embodiment of the present disclosure, the hole transport zone includes a first hole transport layer and a second hole transport layer, wherein the first hole transport layer can be placed between a first electrode and a light-emitting layer, the second hole transport layer can be placed between a first hole transport layer and a light-emitting layer, and wherein the second hole transport layer may be a layer that functions as a hole transport layer, a light-emitting auxiliary layer, a hole auxiliary layer, and/or an electron blocking layer. According to another embodiment of the present disclosure, the hole transport zone includes a first hole transport layer, a second hole transport layer, and a third hole transport layer, wherein the first hole transport layer can be placed between a first electrode and a light-emitting layer, the second hole transport layer can be placed between a first hole transport layer and a light-emitting layer, the third hole transport layer can be placed between a second hole transport layer and a light-emitting layer, and wherein the third hole transport layer may be a layer that functions as a hole transport layer, a light-emitting auxiliary layer, a hole auxiliary layer, and/or an electron blocking layer.

Herein, the term “(C1-C30)alkyl” is meant to be a linear or branched alkyl having 1 to 30 carbon atoms constituting the chain, in which the number of carbon atoms is preferably 1 to 10, and more preferably 1 to 6. The above alkyl may include methyl, ethyl, n-propyl, isopropyl, n-butyl, isobutyl, tert-butyl, sec-butyl, etc. The term “(C3-C30)cycloalkyl” is meant to be a mono-or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3-to 7-membered)heterocycloalkyl” is meant to be a cycloalkyl having 3 to 7 ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl”, “(C6-C30)arylene”, and “(C6-C30)arentriyl” are meant to be a monocyclic or fused ring radical derived from an aromatic hydrocarbon having 6 to 30 ring backbone carbon atoms. The above aryl, arylene, and arentriyl may be partially saturated, and may comprise a spiro structure. The above aryl may include phenyl, biphenyl, terphenyl, quinquephenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, benzophenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluoren]yl, spiro[cyclopentene-fluoren]yl, spiro[dihydroindene-fluoren]yl, azulenyl, tetramethyldihydrophenanthrenyl, etc. Specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenyl, 3-biphenyl, 4-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl) phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl”, “(3- to 30-membered)heteroarylene”, and “(3- to 30-membered)heteroarentriyl” are meant to be an aryl group having 3 to 30 ring backbone atoms, and including at least one, preferably 1 to 4 heteroatoms selected from the group consisting of B, N, O, S, Si, P, Se, Te, and Ge. The above heteroaryl may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, naphthooxazolyl, benzofuroquinolinyl, benzofuroquinazolinyl, benzofuronaphthyridinyl, benzofuropyrimidinyl, naphthofuropyrimidinyl, benzothienoquinolyl, benzothienoquinazolinyl, naphthyridinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, phenanthrooxazolyl, phenanthrothiazolyl, phenanthrobenzofuranyl, benzophenanthrothiophenyl, pyrazinoindolyl, benzopyrazinoindolyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, benzoquinazolinyl, quinoxalinyl, benzoquinoxalinyl, carbazolyl, benzocarbazolyl, dibenzocarbazolyl, phenoxazinyl, phenanthridinyl, benzodioxolyl, dihydroacridinyl, benzotriazolyl, phenazinyl, imidazopyridyl, chromenoquinazolinyl, thiochromenoquinazolinyl, dimethylbenzoperimidinyl, indolocarbazolyl, indenocarbazolyl, etc. More specifically, the above heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl) pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2, 1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2, 1-b]-benzothiophenyl, 3-naphtho-[2,1-b]-benzothiophenyl, 4-naphtho-[2, 1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2, 1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio [3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. “Heteroaryl(ene)” can be classified into heteroaryl(ene) with electronic properties and heteroaryl(ene) with hole properties. Heteroaryl(ene) with electronic properties is a substituent which is relatively rich in electrons in the parent nucleus, for example, a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, or a substituted or unsubstituted quinolyl, etc. Heteroaryl(ene) with hole properties is a substituent which is relatively poor in electrons in the parent nucleus, for example, a substituted or unsubstituted carbazolyl, a substituted or unsubstituted dibenzofuranyl, a substituted or unsubstituted dibenzothiophenyl, etc. Furthermore, “halogen” includes F, Cl, Br, and I.

In addition, “ortho (o-),” “meta (m-),” and “para (p-)” are prefixes, which represent the relative positions of substituents respectively. Ortho indicates that two substituents are adjacent to each other, and for example, when two substituents in a benzene derivative occupy positions 1 and 2 or positions 2 and 3, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, “substituted” in the expression “substituted or unsubstituted” means that a hydrogen atom in a certain functional group is replaced with another atom or another functional group, i.e., a substituent. Unless otherwise specified, the substituent may replace hydrogen at a position where the substituent can be substituted without limitation, and when two or more hydrogen atoms in a certain functional group are each replaced with a substituent, each substituent may be the same or different from each other. The maximum number of substituents that can be substituted for a certain functional group may be the total number of valences that can be substituted for each atom forming the functional group. Herein, the substituted alkyl, the substituted aryl, the substituted heteroaryl, the substituted arylene, the substituted heteroarylene, and the substituted cycloalkyl each independently are substituted with at least one selected from the group consisting of deuterium; a halogen; a cyano; a carboxyl; a nitro; a hydroxy; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered) heteroaryl unsubstituted or substituted with at least one of a (C6-C30)aryl(s); a (C6-C30)aryl unsubstituted or substituted with at least one of a (C1-C30)alkyl(s) and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C2-C30)alkenylamino; a mono- or di-(C6-C30)arylamino unsubstituted or substituted with a (C1-C30)alkyl(s); a mono- or di-(3- to 30-membered)heteroarylamino; a (C1-C30)alkyl(C2-C30)alkenylamino; a (C1-C30)alkyl (C6-C30)arylamino; a (C1-C30)alkyl(3- to 30-membered)heteroarylamino; a (C2-C30)alkenyl(C6-C30)arylamino; a (C2-C30)alkenyl (3- to 30-membered)heteroarylamino; a (C6-C30)aryl(3-to 30-membered)heteroarylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituted alkyl, etc. each independently may be substituted with at least one selected from the group consisting of deuterium; a substituted or unsubstituted (3- to 30-membered)heteroaryl; a substituted or unsubstituted (C6-C30)aryl; an amino; a mono- or di-(C6-C30)arylamino; (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino. According to another embodiment of the present disclosure, the substituted alkyl, etc. each independently may be substituted with at least one selected from the group consisting of a substituted or unsubstituted (3- to 20-membered)heteroaryl; a substituted or unsubstituted (C6-C20)aryl; a mono- or di-(C6-C25)arylamino. For example, the substituted alkyl, etc. each independently may be substituted with a phenyl, a naphthyl, a dibenzofuranyl, a diphenylamino, or a phenylnaphthylamino, and these can be further substituted with deuterium.

In the present disclosure, if a substituent is not indicated in the chemical formula or compound structure, it may mean that all possible positions for the substituent are hydrogen or deuterium. That is, in the case of deuterium, it is an isotope of hydrogen, and some hydrogen atoms may be the isotope deuterium, and in this case, the content of deuterium may be 0% to 100%. In the present disclosure, in cases where a substituent is not indicated in the chemical formula or compound structure, if the deuteriumis not explicitly excluded, such as 0% deuterium, 100% hydrogen, and all substituents are hydrogen, hydrogen and deuterium may be used intermixed in a compound. The deuterium is one of the isotopes of hydrogen and an element with a deuteron consisting of one proton and one neutron as its nucleus. It can be represented as hydrogen-2, whose element symbol can also be written as D or 2H. The isotopes are atoms with the same atomic number (Z) but different mass numbers (A), it can also be interpreted as elements with the same number of protons but different numbers of neutrons.

In the present disclosure, “a combination thereof” refers to a combination of one or more elements from the corresponding list to form a known or chemically stable arrangement that can be envisioned by a person skilled in the art from the corresponding list. For example, alkyl and deuterium can be combined to form a partially or fully deuterated alkyl group; halogen and alkyl can be combined to form a halogenated alkyl substituent; halogen, alkyl, and aryl can be combined to form a halogenated arylalkyl. For example, a preferred combination of substituents include up to 50 atoms that are not hydrogen or deuterium, or up to 40 atoms that are not hydrogen or deuterium, or up to 30 atoms that are not hydrogen or deuterium, or in many cases, a preferred combination of substituents may comprise up to 20 atoms that are not hydrogen or deuterium.

In the formulas of the present disclosure, when a ring is formed by a linkage of adjacent substituents, the ring may be a substituted or unsubstituted, mono- or polycyclic, (3- to 30-membered) alicyclic or aromatic ring, or a combination thereof, which is formed by linkage of at least two adjacent substituents. In addition, the formed ring may contain at least one heteroatom selected from B, N, O, S, Si, and P, preferably at least one heteroatom selected from N, O, and S. According to one embodiment of the present disclosure, the number of the ring backbone atoms is 5 to 20, and according to another embodiment of the present disclosure, the number of the ring backbone atoms is 5 to 15.

The present disclosure provides the organic electroluminescent compound represented by formula 1.

Hereinafter, the compound represented by formula 1 will be described in more detail.

In formula 1, X represents NR₁, O, S, Se, CR₂R₃, or SiR₄R₅. According to one embodiment of the present disclosure, X represents O or S. For example, X may represent O.

In formula 1, R₁ to R₅ each independently represent a substituted or unsubstituted (C1-C30)alkyl, a substituted or unsubstituted (C6-C30)aryl, or a substituted or unsubstituted (3- to 30-membered) heteroaryl. According to one embodiment of the present disclosure, R₁ to R₅ each independently represent a substituted or unsubstituted (C1-C30)alkyl, or a substituted or unsubstituted (C6-C30)aryl.

In formula 1, Y represents O, S, Se, or Te. According to one embodiment of the present disclosure, Y represents O, S, or Se. For example, Y may represent O or S.

In formula 1, Z₁ to Z₆ each independently represent N or CR₁₁. For example, Z₁ to Z₆ each independently may represent CR₁₁.

In formula 1, R₁₁ represents hydrogen, deuterium, or -L₁—R₁₃. For example, R₁₁ may represent hydrogen or -L₁—R₁₃.

In formula 1, R₁₂ represents -L₂—R₁₄.

In formula 1, L₁ and L₂ each independently represent a single bond, a substituted or unsubstituted (C6-C30)arylene, or a substituted or unsubstituted (3- to 30-membered)heteroarylene. According to one embodiment of the present disclosure, L₁ and L₂ each independently represent a single bond, a substituted or unsubstituted (C6-C20)arylene, or a substituted or unsubstituted (3- to 20-membered)heteroarylene. According to another embodiment of the present disclosure, L₁ and L₂ each independently represent a single bond, or a substituted or unsubstituted (C6-C20)arylene. For example, L₁ and L₂ each independently may be a single bond or a phenylene.

In formula 1, R₁₃ and R₁₄ each independently represent a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, -L′₁—SiR′₁R′₂R′₃, -L′₂—GeR′₄R′₅R′₆, -L′₃—NR′₇R′₈, -L′₄(NR′₉R′₁₀)(NR′₁₁R′₁₂), -L′₅—NR′₁₃R′₁₄, or -L′₆—(NR′₁₅), -L′₇(NR′₁₆R′₁₇). According to one embodiment of the present disclosure, R₁₃ and R₁₄ each independently represent a substituted or unsubstituted (C1-C20)alkyl, a substituted or unsubstituted (C6-C20)aryl, a substituted or unsubstituted (3- to 20-membered)heteroaryl, a substituted or unsubstituted (C3-C20)cycloalkyl, -L′₃—NR′₇R′₈, -L′₄(NR′₉R′₁₀)(NR′₁₁R′₁₂), —L′₅—NR′₁₃R′₁₄, or -L′₆—(NR′₁₅)—L′₇(NR′₁₆R′₁₇). According to another embodiment of the present disclosure, R₁₃ and R₁₄ each independently represent a substituted or unsubstituted (C6-C20)aryl, a substituted or unsubstituted (3- to 20-membered)heteroaryl, -L′₃—NR′₇R′₈, -L′₄(NR′₉R′₁₀)(NR′₁₁R′₁₂), -L′₅—NR′₁₃R′₁₄, or -L′₆—(NR′₁₅)—L′₇(NR′₁₆R′₁₇). For example, R₁₃ and R₁₄ each independently may be a phenyl, -L′₃—NR′₇R′₈, -L′₄(NR′₉R′₁₀)(NR′₁₁R′₁₂), -L′₅—NR′₁₃R′₁₄, or -L′₆—(NR′₁₅)—L′₇(NR′₁₆R′₁₇).

In formula 1, L′₁ to L′₇ each independently represent a single bond, a substituted or unsubstituted (C6-C30)arylene, or a substituted or unsubstituted (3- to 30-membered) heteroarylene. According to one embodiment of the present disclosure, L′₁ to L′₇ each independently represent a single bond, or a substituted or unsubstituted (C6-C30)arylene. According to another embodiment of the present disclosure, L′₁ to L′₇ each independently represent a single bond, or a substituted or unsubstituted (C6-C25)arylene. For example, L′₁ to L′₇ each independently may be a single bond, a phenylene, or dimethylfluorenylene.

In formula 1, R′₁ to R′₁₇ each independently represent a substituted or unsubstituted (C1-C30)alkyl, a substituted or unsubstituted (C6-C30)aryl, a substituted or unsubstituted (3- to 30-membered) heteroaryl, or a substituted or unsubstituted (C3-C30)cycloalkyl. According to one embodiment of the present disclosure, R′₁ to R′₁₇ each independently represent a substituted or unsubstituted (C1-C25)alkyl, a substituted or unsubstituted (C6-C25)aryl, a substituted or unsubstituted (3- to 25-membered)heteroaryl, or a substituted or unsubstituted (C3-C25)cycloalkyl. For example, R′₁ to R′₁₇ each independently may be a phenyl unsubstituted or substituted with a naphthyl(s), a diphenylamino(s), or a phenylnaphthylamino(s); a biphenyl; a dibenzofuranyl; a dibenzothiophenyl; a dimethylfluorenyl unsubstituted or substituted with a diphenylamino(s); a phenanthrenyl; a terphenyl; a naphthyl; or a carbazolyl substituted with a phenyl(s).

According to one embodiment of the present disclosure, formula 1 may be represented by one of the following formulas 1-1 to 1-3.

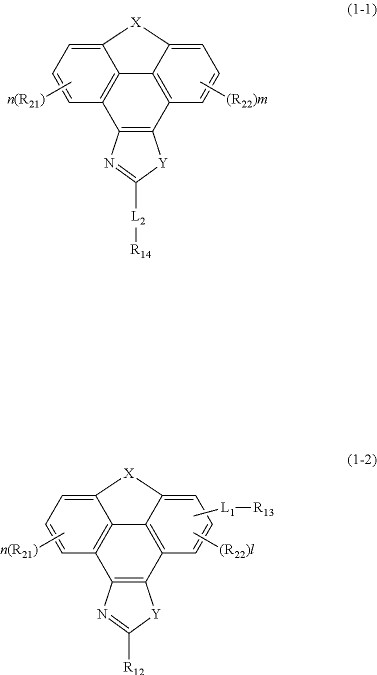

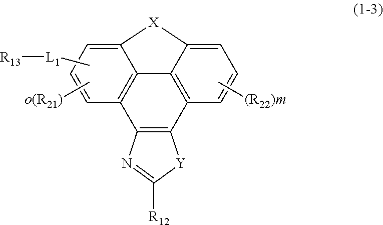

In formulas 1-1 to 1-3,

R₂₁ and R₂₂ each independently represent hydrogen, deuterium, or a (C6-C30)aryl unsubstituted or substituted with deuterium. According to one embodiment of the present disclosure, R₂₁ and R₂₂ each independently represent hydrogen, deuterium, or a (C6-C25)aryl unsubstituted or substituted with deuterium. For example, R₂₁ and R₂₂ may be hydrogen.

n and m each independently represent an integer of 3, l and o each independently represent an integer of 2, and each of R₂₁ and each of R₂₂ may be the same as or different from each other.

L₁, L₂, X, Y, and R₁₂ to R₁₄ are as defined in formula 1.

According to one embodiment of the present disclosure, formula 1 may be represented by one of the following formulas 1-11 to 1-21.

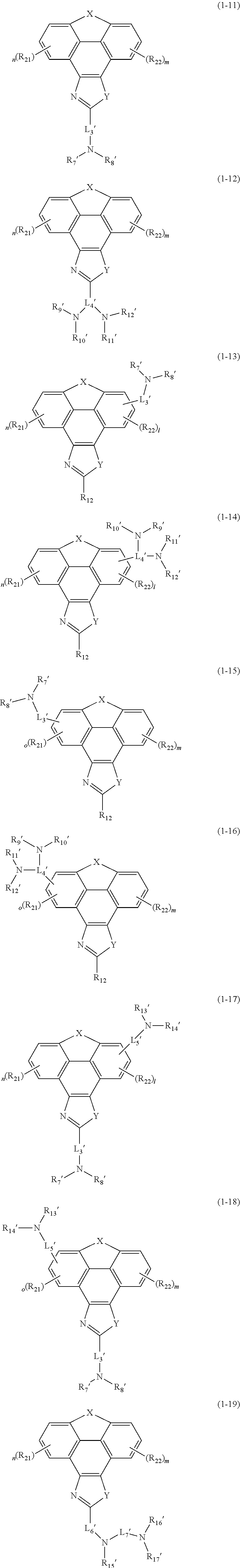

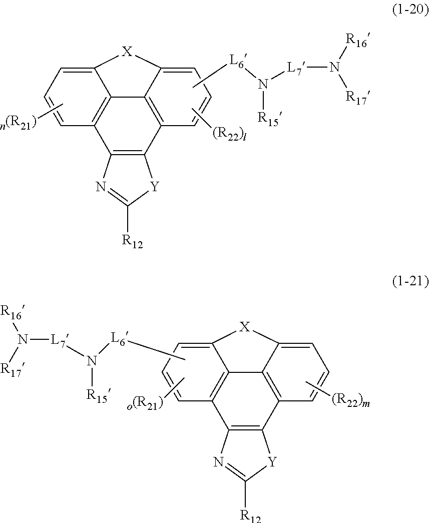

In formulas 1-11 to 1-21,

R₂₁ and R₂₂ each independently represent hydrogen, deuterium, or a (C6-C30)aryl unsubstituted or substituted with deuterium. According to one embodiment of the present disclosure, R₂₁ and R₂₂ each independently represent hydrogen, deuterium, or a (C6-C25) aryl unsubstituted or substituted with deuterium. For example, R₂₁ and R₂₂ may be hydrogen.

n and m each independently represent an integer of 3, l and o each independently represent an integer of 2, and each of R₂₁ and each of R₂₂ may be the same as or different from each other.

X, Y, L′₂ to L′₇, R′₇ to R′₁₇, and R₁₂ are as defined in formula 1.

According to one embodiment of the present disclosure, R′₇ to R′₁₇ each independently may be selected from a phenyl, a biphenyl, a terphenyl, a quinquiphenyl, a naphthyl, a binaphthyl, a phenylnaphthyl, a naphthylphenyl, a chrysenyi, a fluorenyi, a dimethylfluorenyl, a diethylfluorenyl, a methylphenyifluorenyl, a diphenylfluorenyl, a phenanthrenyl, a dibenzofuranyl, a dibenzothiophenyl, a pyridyi, a carbazolyl, or a combination thereof, and the selected substituents may be partially or fully substituted with deuterium.

According to one embodiment of the present disclosure, formula 1 may be represented by one of the following formulas 1-31 to 1-33.

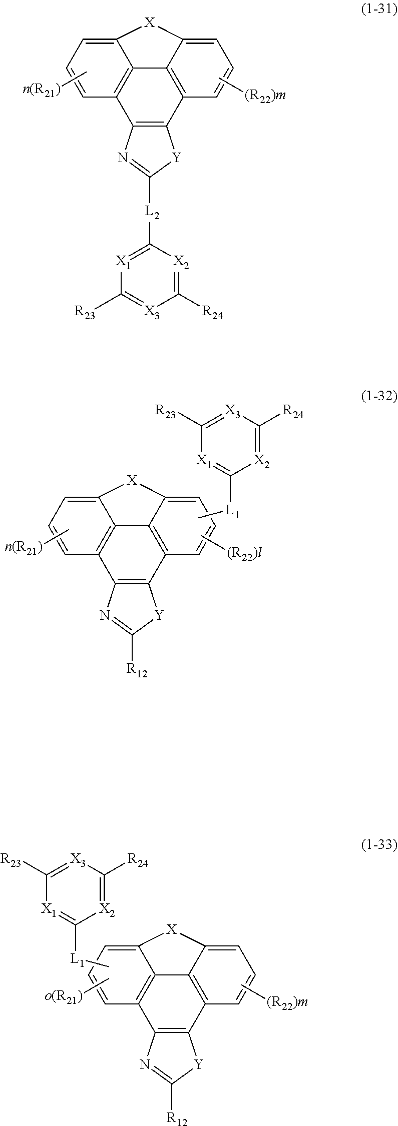

In formulas 1-31 to 1-33,

X₁ to X₃ each independently represent N, CH, or CD, and at least one of these is N. For example, X₁ to X₃ each independently may be N.

R₂₃ and R₂₄ each independently represent a substituted or unsubstituted (C6-C30)aryl, or a substituted or unsubstituted (3- to 30-membered) heteroaryl. According to one embodiment of the present disclosure, R₂₃ and R₂₄ each independently represent a substituted or unsubstituted (C6-C25)aryl, or a substituted or unsubstituted (3- to 25-membered) heteroaryl. According to another embodiment of the present disclosure, R₂₃ and R₂₄ each independently are selected from a phenyl, a biphenyl, a terphenyl, a quinquiphenyl, a naphthyl, a binaphthyl, a phenylnaphthyl, a naphthylphenyl, a chrysenyl, a fluorenyl, a dimethylfluorenyl, a methylphenylfluorenyl, a diphenylfluorenyl, a phenanthrenyl, a dibenzofuranyl, a dibenzothiophenyl, a pyridyl, a carbazolyl, or a combination thereof, and the selected substituents may be partially or fully substituted with deuterium. For example, R₂₃ and R₂₄ each independently may be a phenyl, a biphenyl, a naphthyl, a phenanthrenyl, a chrysenyl, a dibenzofuranyl, or a dibenzothiophenyl.

R₂₁ and R₂₂ each independently represent hydrogen, deuterium, or a (C6-C30)aryl. According to one embodiment of the present disclosure, R₂₁ and R₂₂ each independently represent hydrogen, deuterium, or a (C6-C25)aryl. For example, R₂₁ and R₂₂ may be hydrogen.

n and m each independently represent an integer of 3, l and o each independently represent an integer of 2, and each of R₂₁ and each of R₂₂ may be the same as or different from each other.

X, Y, L₁, L₂, and R₁₂ are as defined in formula 1.

The compound represented by formula 1 may be selected from the group consisting of the following compounds, but is not limited thereto.

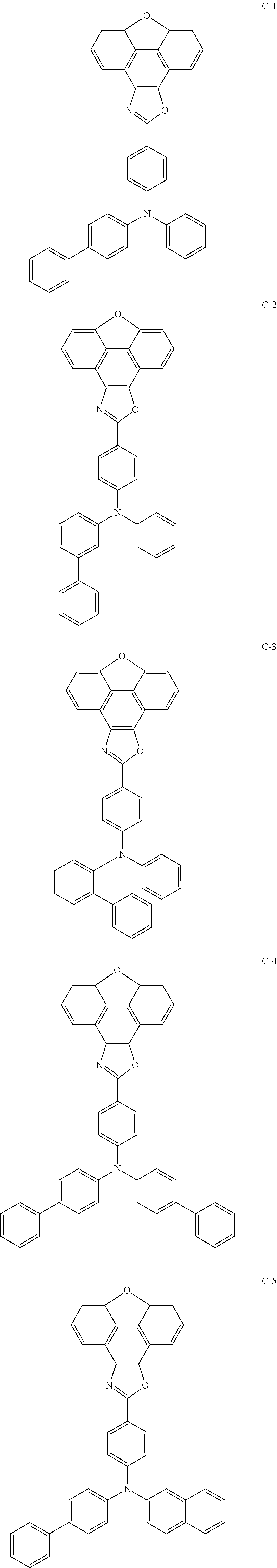 [SMILES: c1ccc(-c2ccc(N(c3ccccc3)c3ccc(-c4nc5c6cccc7oc8cccc(c5o4)c8c76)cc3)cc2)cc1 ; c1ccc(-c2cccc(N(c3ccccc3)c3ccc(-c4nc5c6cccc7oc8cccc(c5o4)c8c76)cc3)c2)cc1 ; c1ccc(-c2ccccc2N(c2ccccc2)c2ccc(-c3nc4c5cccc6oc7cccc(c4o3)c7c65)cc2)cc1 ; c1ccc(-c2ccc(N(c3ccc(-c4ccccc4)cc3)c3ccc(-c4nc5c6cccc7oc8cccc(c5o4)c8c76)cc3)cc2)cc1 ; c1ccc(-c2ccc(N(c3ccc(-c4nc5c6cccc7oc8cccc(c5o4)c8c76)cc3)c3ccc4ccccc4c3)cc2)cc1]

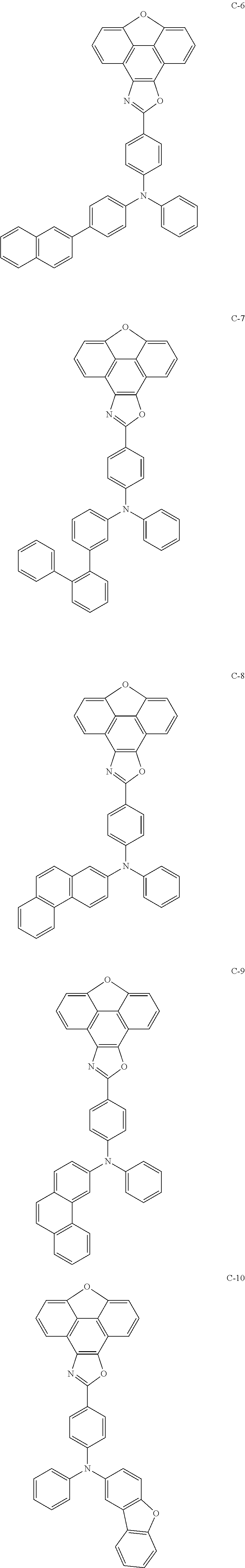

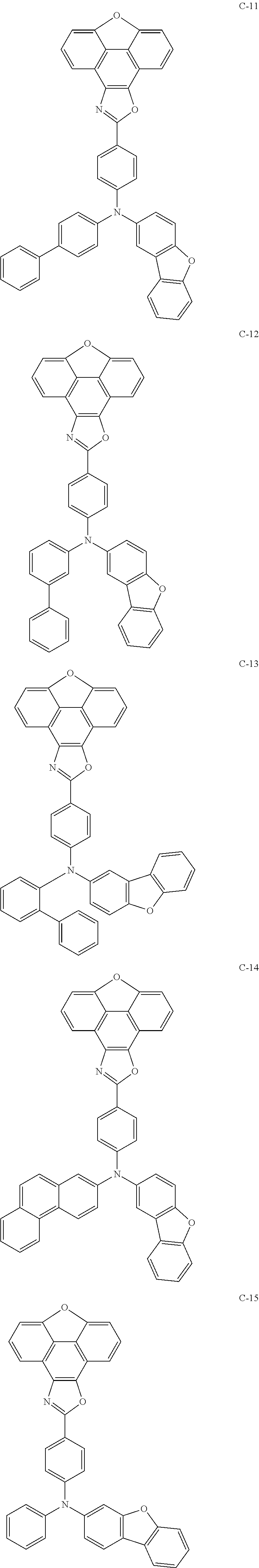

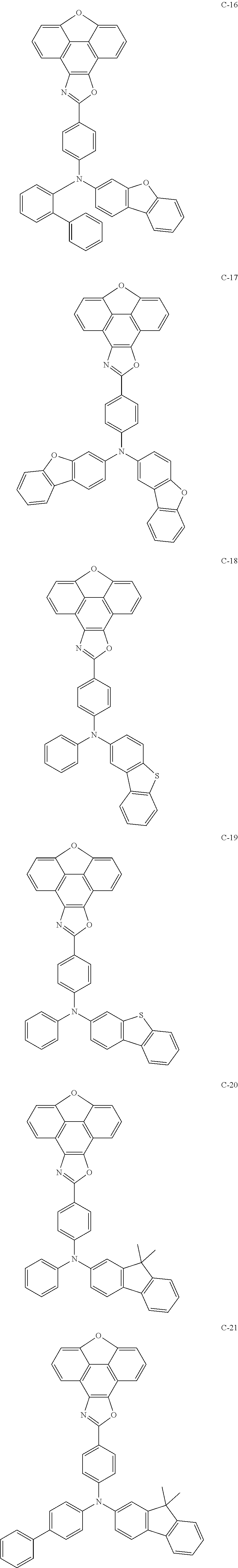 [SMILES: c1ccc(-c2ccccc2N(c2ccc(-c3nc4c5cccc6oc7cccc(c4o3)c7c65)cc2)c2ccc3c(c2)oc2ccccc23)cc1 ; c1ccc2c(c1)oc1cc(N(c3ccc(-c4nc5c6cccc7oc8cccc(c5o4)c8c76)cc3)c3ccc4oc5ccccc5c4c3)ccc12 ; c1ccc(N(c2ccc(-c3nc4c5cccc6oc7cccc(c4o3)c7c65)cc2)c2ccc3sc4ccccc4c3c2)cc1 ; c1ccc(N(c2ccc(-c3nc4c5cccc6oc7cccc(c4o3)c7c65)cc2)c2ccc3c(c2)sc2ccccc23)cc1 ; CC1(C)c2ccccc2-c2ccc(N(c3ccccc3)c3ccc(-c4nc5c6cccc7oc8cccc(c5o4)c8c76)cc3)cc21 ; CC1(C)c2ccccc2-c2ccc(N(c3ccc(-c4ccccc4)cc3)c3ccc(-c4nc5c6cccc7oc8cccc(c5o4)c8c76)cc3)cc21]

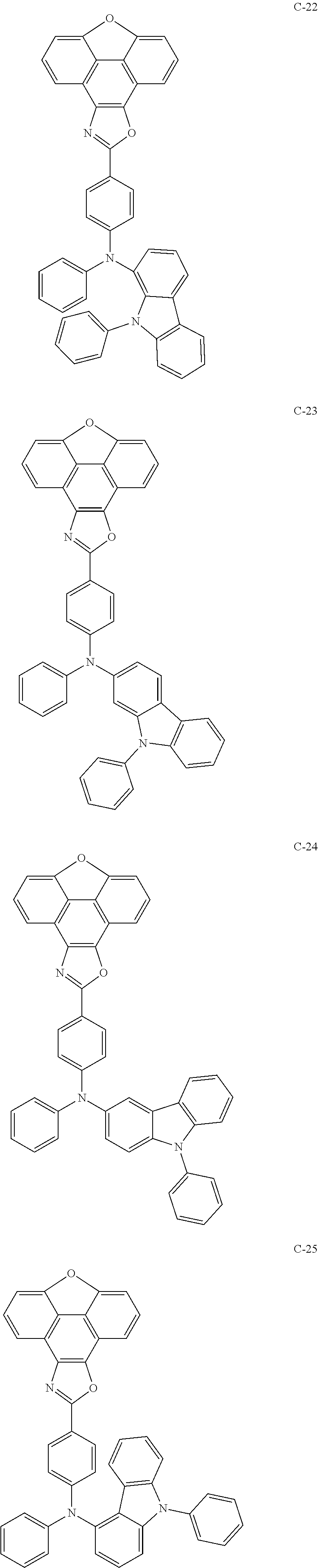 [SMILES: c1ccc(N(c2ccc(-c3nc4c5cccc6oc7cccc(c4o3)c7c65)cc2)c2cccc3c4ccccc4n(-c4ccccc4)c23)cc1 ; c1ccc(N(c2ccc(-c3nc4c5cccc6oc7cccc(c4o3)c7c65)cc2)c2ccc3c4ccccc4n(-c4ccccc4)c3c2)cc1 ; c1ccc(N(c2ccc(-c3nc4c5cccc6oc7cccc(c4o3)c7c65)cc2)c2ccc3c(c2)c2ccccc2n3-c2ccccc2)cc1 ; c1ccc(N(c2ccc(-c3nc4c5cccc6oc7cccc(c4o3)c7c65)cc2)c2cccc3c2c2ccccc2n3-c2ccccc2)cc1]

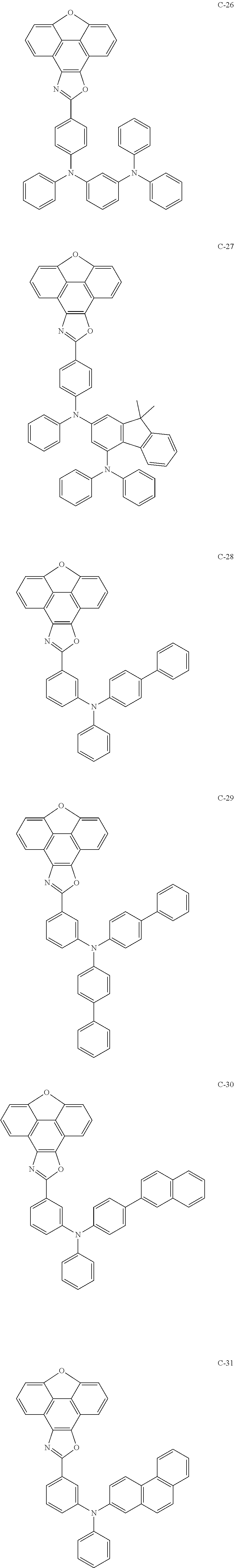

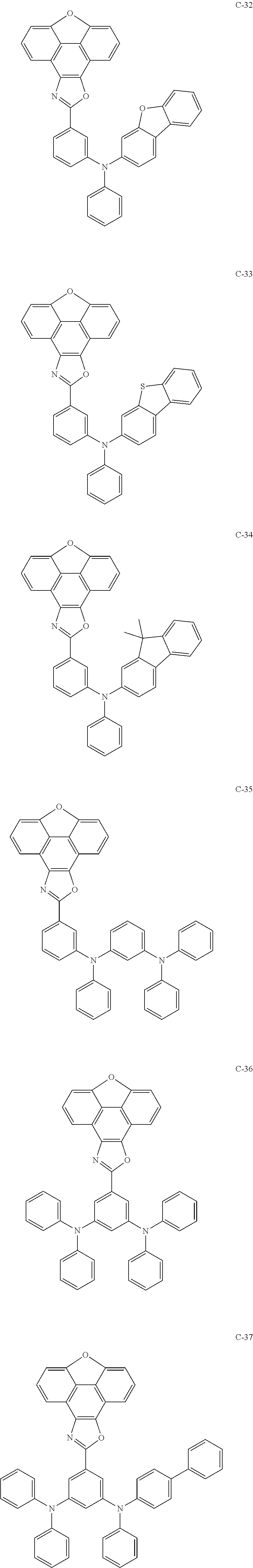

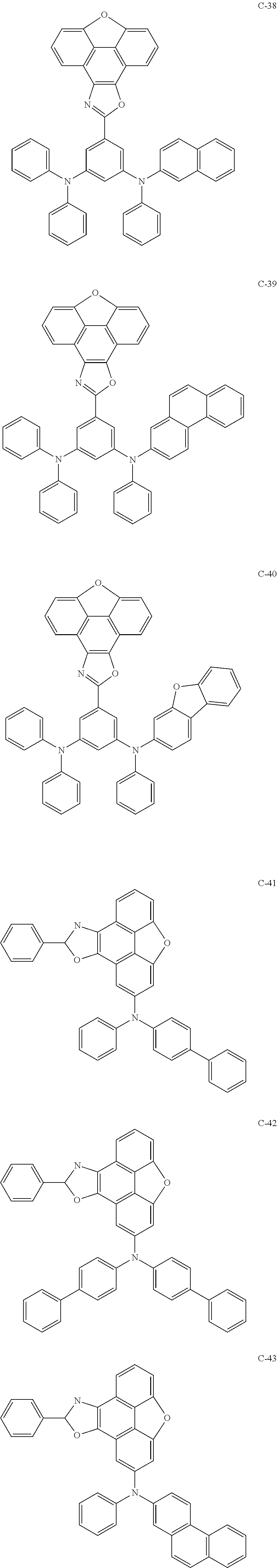

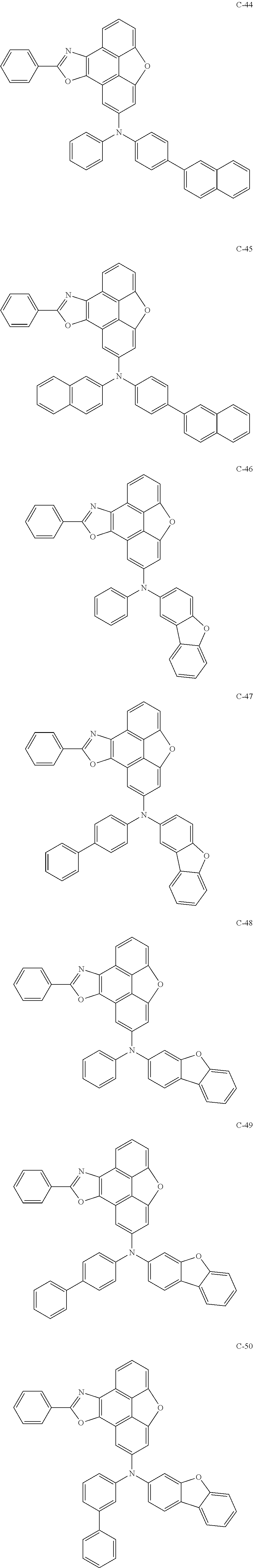

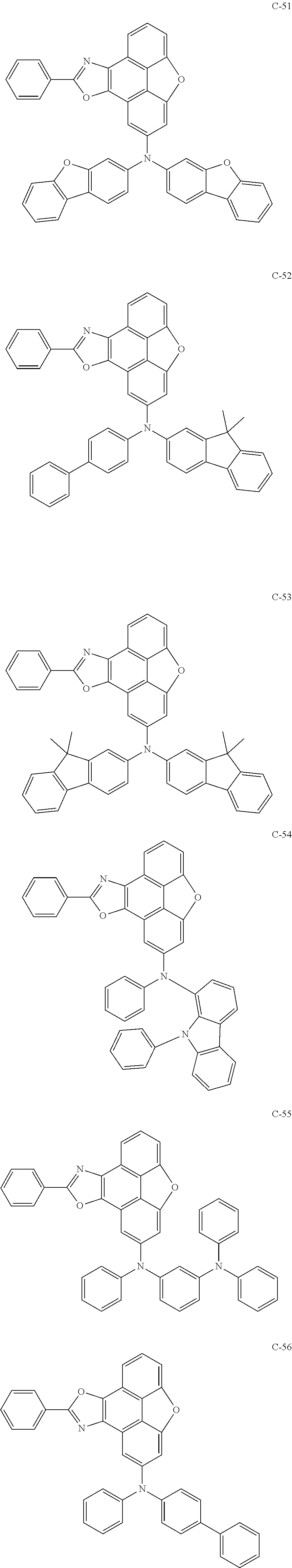

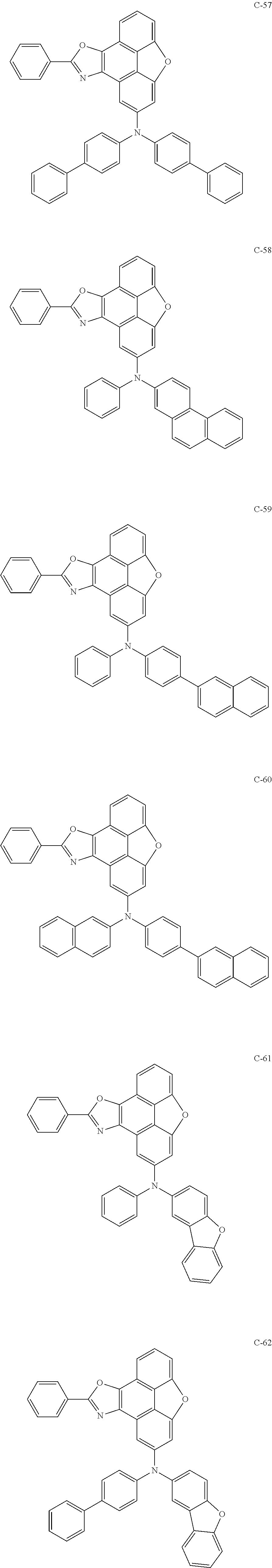 [SMILES: c1ccc(-c2ccc(N(c3ccc(-c4ccccc4)cc3)c3cc4oc5cccc6c7oc(-c8ccccc8)nc7c(c3)c4c56)cc2)cc1 ; c1ccc(-c2nc3c4cc(N(c5ccccc5)c5ccc6c(ccc7ccccc76)c5)cc5oc6cccc(c3o2)c6c54)cc1 ; c1ccc(-c2nc3c4cc(N(c5ccccc5)c5ccc(-c6ccc7ccccc7c6)cc5)cc5oc6cccc(c3o2)c6c54)cc1 ; c1ccc(-c2nc3c4cc(N(c5ccc(-c6ccc7ccccc7c6)cc5)c5ccc6ccccc6c5)cc5oc6cccc(c3o2)c6c54)cc1 ; c1ccc(-c2nc3c4cc(N(c5ccccc5)c5ccc6oc7ccccc7c6c5)cc5oc6cccc(c3o2)c6c54)cc1 ; c1ccc(-c2ccc(N(c3ccc4oc5ccccc5c4c3)c3cc4oc5cccc6c7oc(-c8ccccc8)nc7c(c3)c4c56)cc2)cc1]

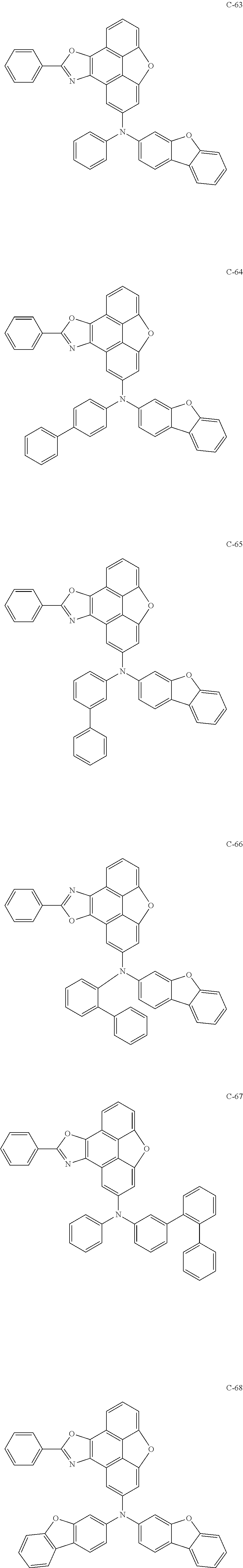

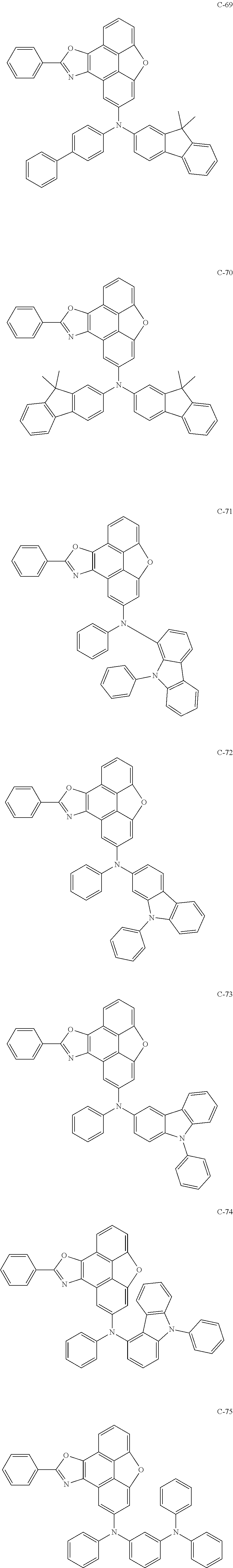

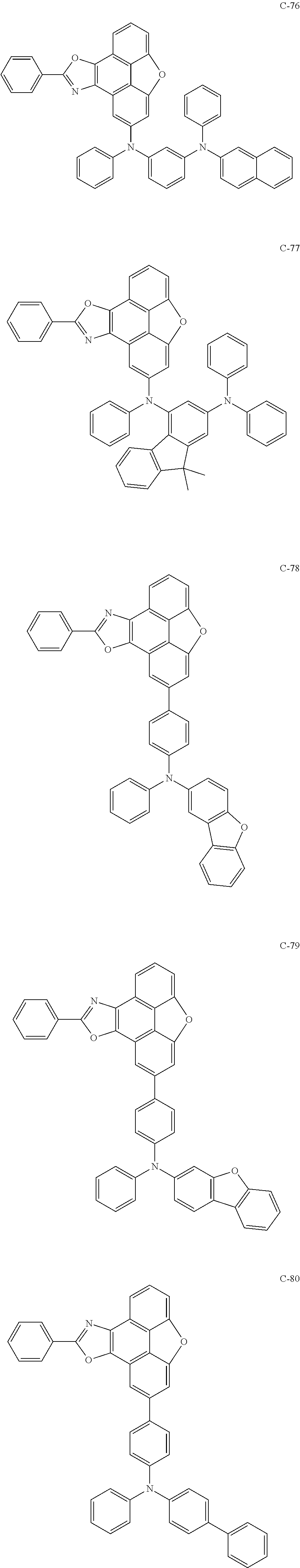 [SMILES: c1ccc(-c2nc3c4cc(N(c5ccccc5)c5cccc(N(c6ccccc6)c6ccc7ccccc7c6)c5)cc5oc6cccc(c3o2)c6c54)cc1 ; CC1(C)c2ccccc2-c2c(N(c3ccccc3)c3cc4oc5cccc6c7oc(-c8ccccc8)nc7c(c3)c4c56)cc(N(c3ccccc3)c3ccccc3)cc21 ; c1ccc(-c2nc3c4cccc5oc6cc(-c7ccc(N(c8ccccc8)c8ccc9oc%10ccccc%10c9c8)cc7)cc(c3o2)c6c54)cc1 ; c1ccc(-c2nc3c4cccc5oc6cc(-c7ccc(N(c8ccccc8)c8ccc9c(c8)oc8ccccc89)cc7)cc(c3o2)c6c54)cc1 ; c1ccc(-c2ccc(N(c3ccccc3)c3ccc(-c4cc5oc6cccc7c8nc(-c9ccccc9)oc8c(c4)c5c67)cc3)cc2)cc1]

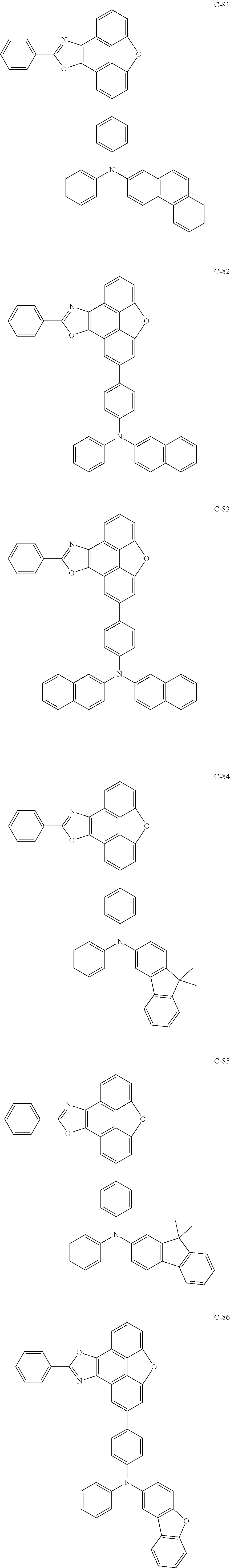

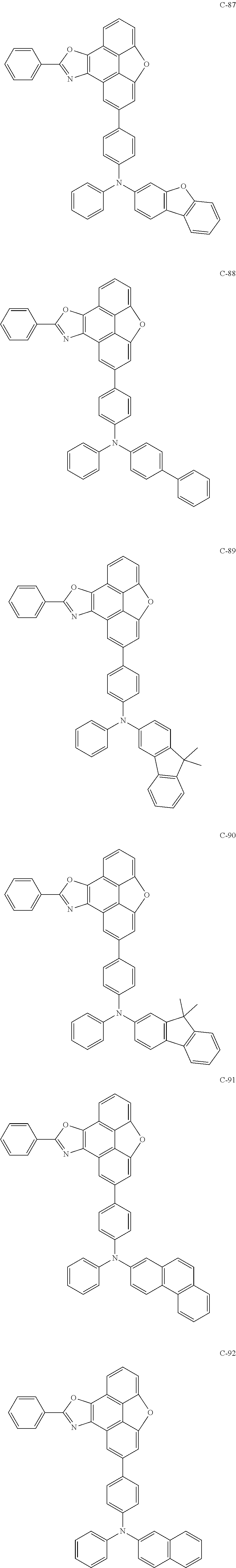

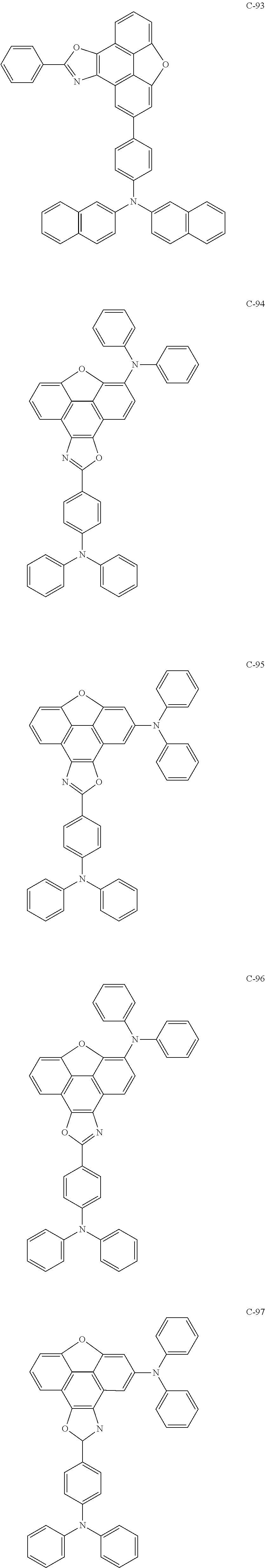

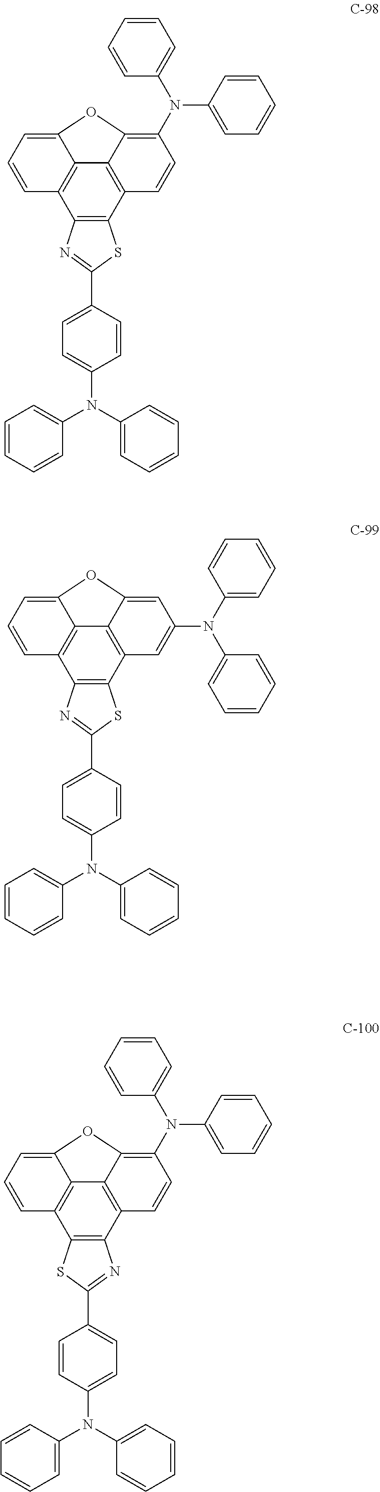 [SMILES: c1ccc(N(c2ccccc2)c2ccc(-c3nc4c5cccc6oc7c(N(c8ccccc8)c8ccccc8)ccc(c4s3)c7c65)cc2)cc1 ; c1ccc(N(c2ccccc2)c2ccc(-c3nc4c5cccc6oc7cc(N(c8ccccc8)c8ccccc8)cc(c4s3)c7c65)cc2)cc1 ; c1ccc(N(c2ccccc2)c2ccc(-c3nc4c5ccc(N(c6ccccc6)c6ccccc6)c6oc7cccc(c4s3)c7c65)cc2)cc1]

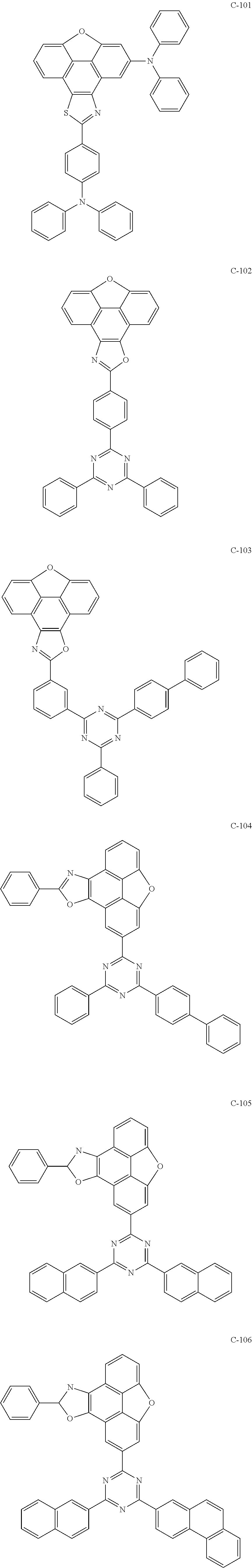

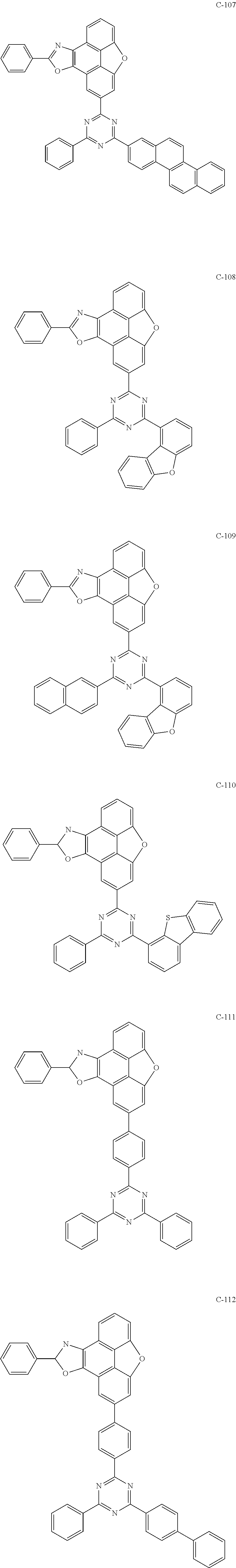

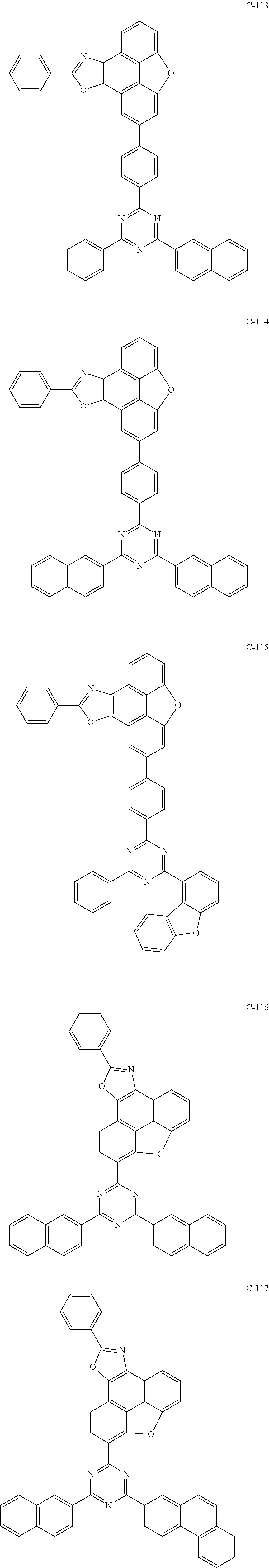 [SMILES: c1ccc(-c2nc(-c3ccc(-c4cc5oc6cccc7c8nc(-c9ccccc9)oc8c(c4)c5c67)cc3)nc(-c3ccc4ccccc4c3)n2)cc1 ; c1ccc(-c2nc3c4cccc5oc6cc(-c7ccc(-c8nc(-c9ccc%10ccccc%10c9)nc(-c9ccc%10ccccc%10c9)n8)cc7)cc(c3o2)c6c54)cc1 ; c1ccc(-c2nc(-c3ccc(-c4cc5oc6cccc7c8nc(-c9ccccc9)oc8c(c4)c5c67)cc3)nc(-c3cccc4oc5ccccc5c34)n2)cc1 ; c1ccc(-c2nc3c4cccc5oc6c(-c7nc(-c8ccc9ccccc9c8)nc(-c8ccc9ccccc9c8)n7)ccc(c3o2)c6c54)cc1 ; c1ccc(-c2nc3c4cccc5oc6c(-c7nc(-c8ccc9ccccc9c8)nc(-c8ccc9c(ccc%10ccccc%109)c8)n7)ccc(c3o2)c6c54)cc1]

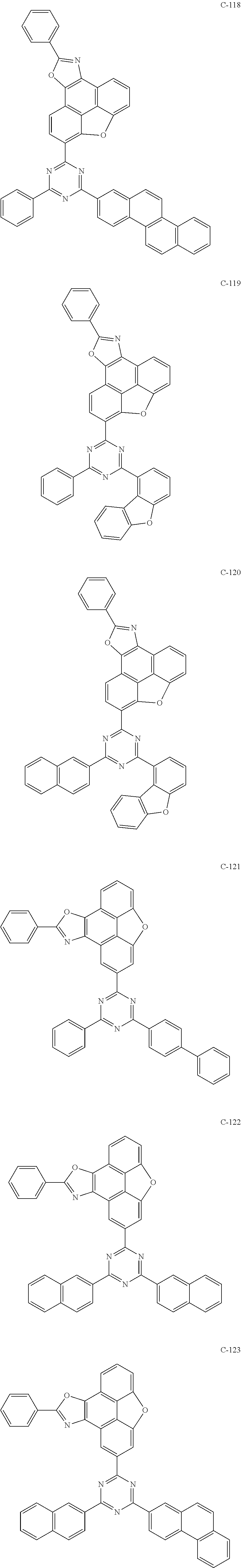

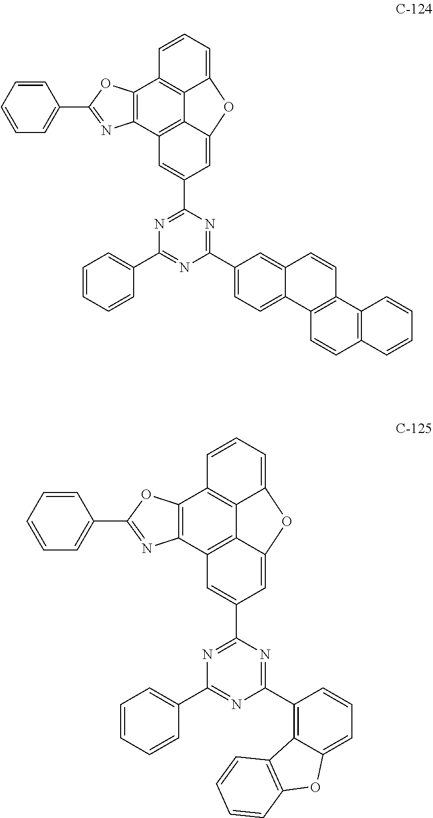 [SMILES: c1ccc(-c2nc(-c3ccc4c(ccc5c6ccccc6ccc45)c3)nc(-c3cc4oc5cccc6c7oc(-c8ccccc8)nc7c(c3)c4c56)n2)cc1 ; c1ccc(-c2nc(-c3cc4oc5cccc6c7oc(-c8ccccc8)nc7c(c3)c4c56)nc(-c3cccc4oc5ccccc5c34)n2)cc1]

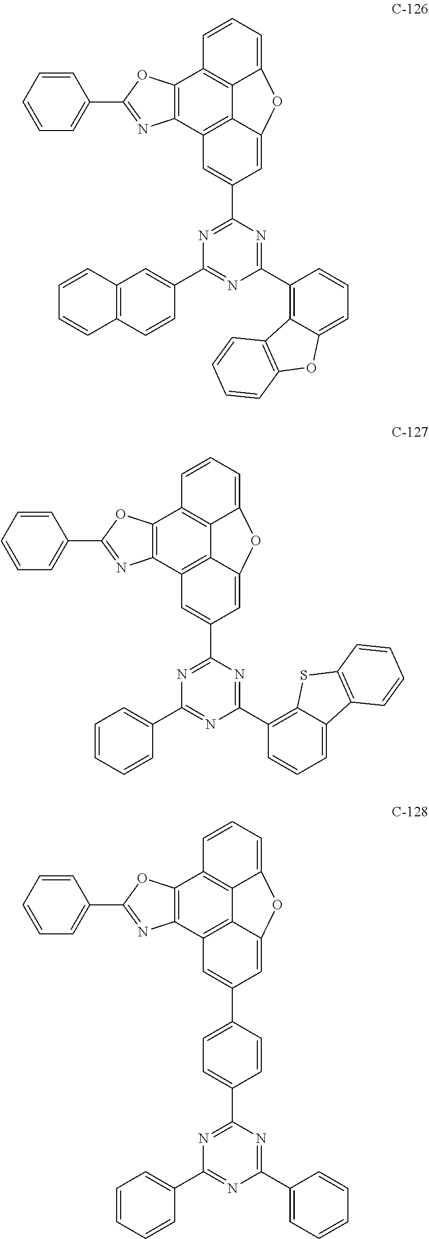 [SMILES: c1ccc(-c2nc3c4cc(-c5nc(-c6ccc7ccccc7c6)nc(-c6cccc7oc8ccccc8c67)n5)cc5oc6cccc(c3o2)c6c54)cc1 ; c1ccc(-c2nc(-c3cc4oc5cccc6c7oc(-c8ccccc8)nc7c(c3)c4c56)nc(-c3cccc4c3sc3ccccc34)n2)cc1 ; c1ccc(-c2nc(-c3ccccc3)nc(-c3ccc(-c4cc5oc6cccc7c8oc(-c9ccccc9)nc8c(c4)c5c67)cc3)n2)cc1]

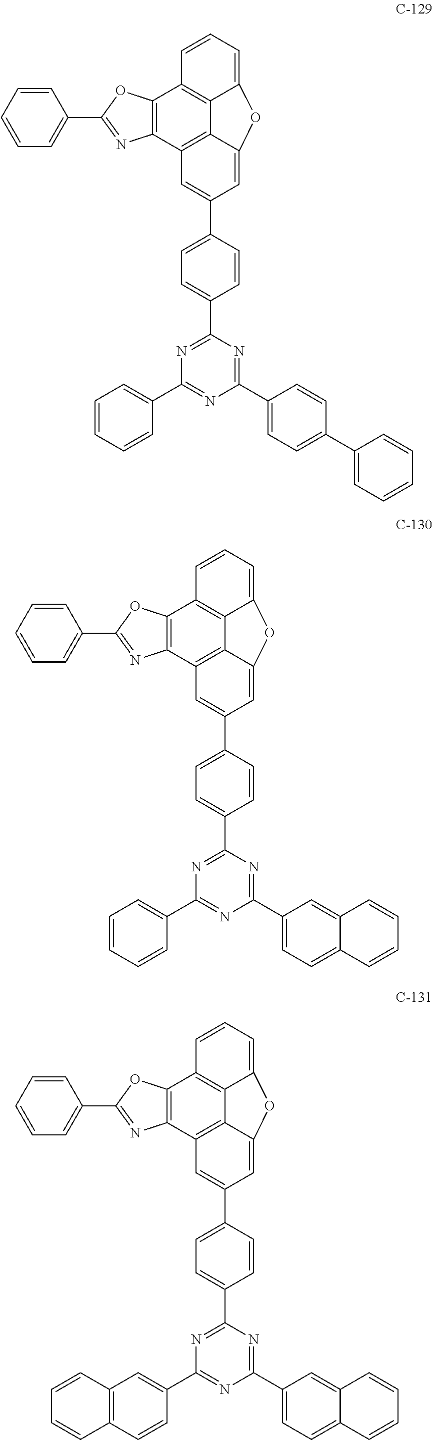 [SMILES: c1ccc(-c2ccc(-c3nc(-c4ccccc4)nc(-c4ccc(-c5cc6oc7cccc8c9oc(-c%10ccccc%10)nc9c(c5)c6c78)cc4)n3)cc2)cc1 ; c1ccc(-c2nc(-c3ccc(-c4cc5oc6cccc7c8oc(-c9ccccc9)nc8c(c4)c5c67)cc3)nc(-c3ccc4ccccc4c3)n2)cc1 ; c1ccc(-c2nc3c4cc(-c5ccc(-c6nc(-c7ccc8ccccc8c7)nc(-c7ccc8ccccc8c7)n6)cc5)cc5oc6cccc(c3o2)c6c54)cc1]

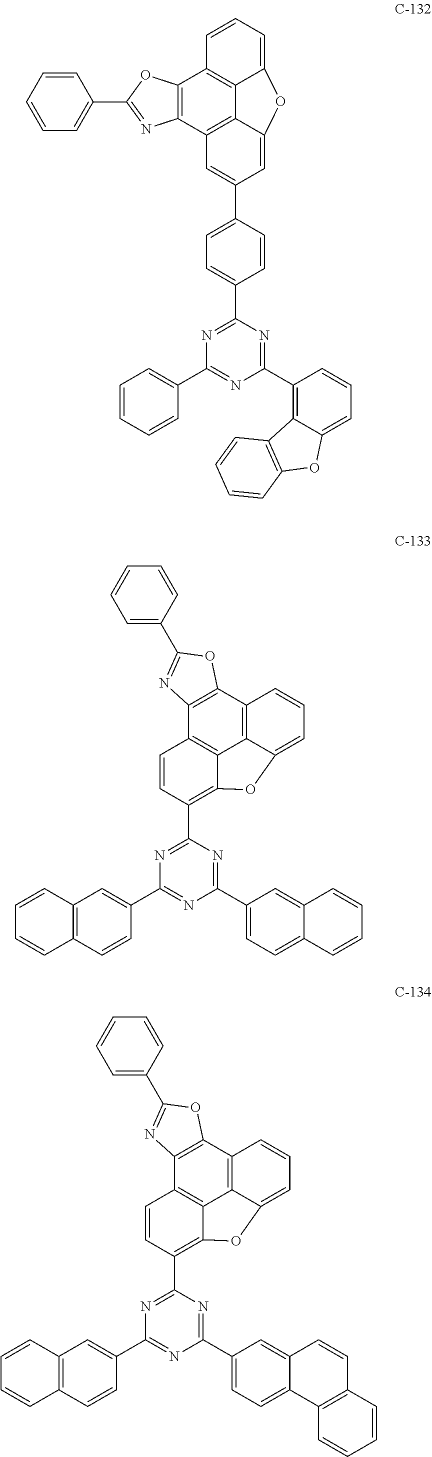 [SMILES: c1ccc(-c2nc(-c3ccc(-c4cc5oc6cccc7c8oc(-c9ccccc9)nc8c(c4)c5c67)cc3)nc(-c3cccc4oc5ccccc5c34)n2)cc1 ; c1ccc(-c2nc3c4ccc(-c5nc(-c6ccc7ccccc7c6)nc(-c6ccc7ccccc7c6)n5)c5oc6cccc(c3o2)c6c54)cc1 ; c1ccc(-c2nc3c4ccc(-c5nc(-c6ccc7ccccc7c6)nc(-c6ccc7c(ccc8ccccc87)c6)n5)c5oc6cccc(c3o2)c6c54)cc1]

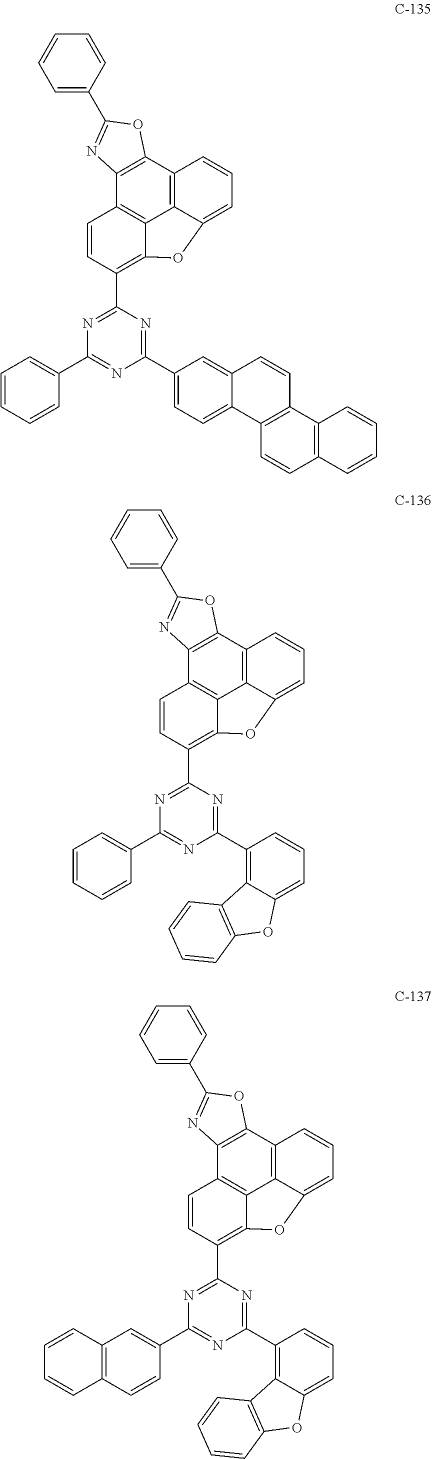 [SMILES: c1ccc(-c2nc(-c3ccc4c(ccc5c6ccccc6ccc45)c3)nc(-c3ccc4c5nc(-c6ccccc6)oc5c5cccc6oc3c4c65)n2)cc1 ; c1ccc(-c2nc(-c3ccc4c5nc(-c6ccccc6)oc5c5cccc6oc3c4c65)nc(-c3cccc4oc5ccccc5c34)n2)cc1 ; c1ccc(-c2nc3c4ccc(-c5nc(-c6ccc7ccccc7c6)nc(-c6cccc7oc8ccccc8c67)n5)c5oc6cccc(c3o2)c6c54)cc1]

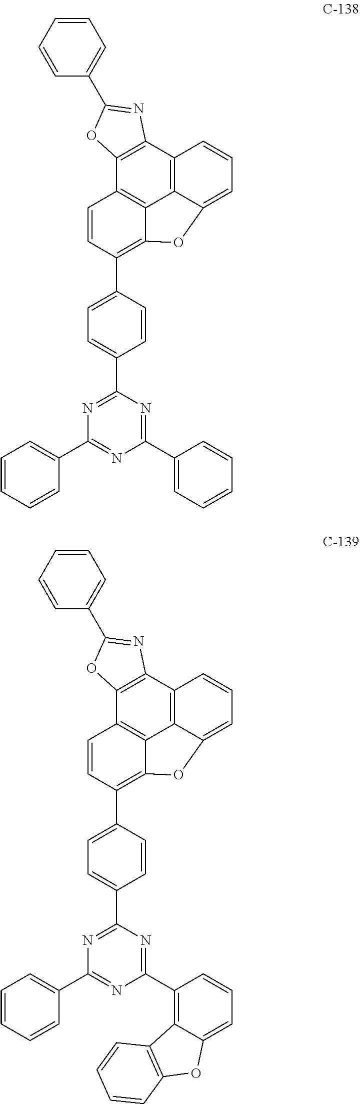 [SMILES: c1ccc(-c2nc(-c3ccccc3)nc(-c3ccc(-c4ccc5c6oc(-c7ccccc7)nc6c6cccc7oc4c5c76)cc3)n2)cc1 ; c1ccc(-c2nc(-c3ccc(-c4ccc5c6oc(-c7ccccc7)nc6c6cccc7oc4c5c76)cc3)nc(-c3cccc4oc5ccccc5c34)n2)cc1]

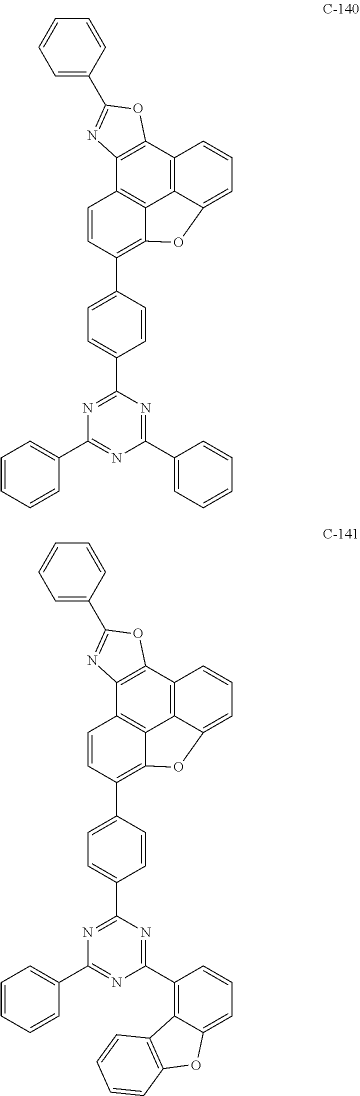

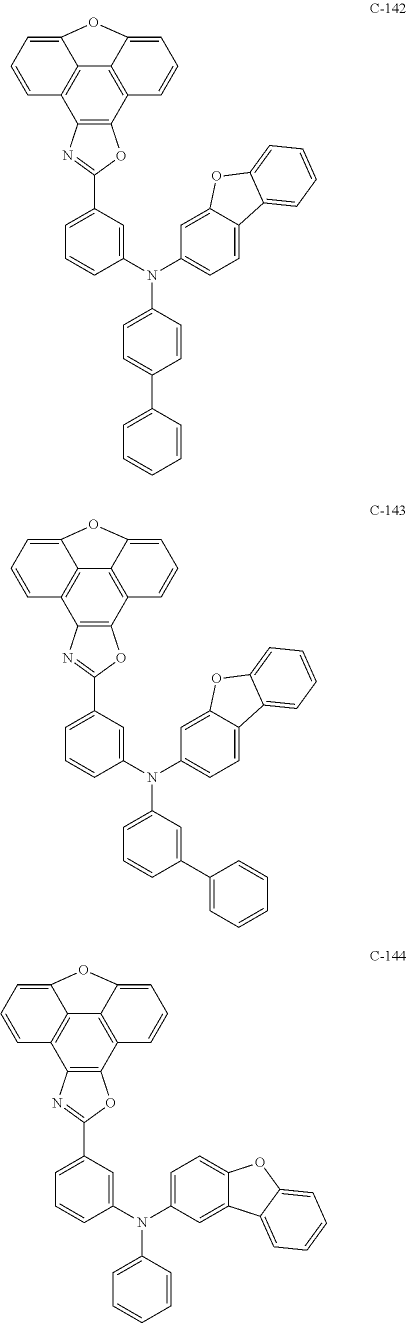 [SMILES: c1ccc(-c2ccc(N(c3cccc(-c4nc5c6cccc7oc8cccc(c5o4)c8c76)c3)c3ccc4c(c3)oc3ccccc34)cc2)cc1 ; c1ccc(-c2cccc(N(c3cccc(-c4nc5c6cccc7oc8cccc(c5o4)c8c76)c3)c3ccc4c(c3)oc3ccccc34)c2)cc1 ; c1ccc(N(c2cccc(-c3nc4c5cccc6oc7cccc(c4o3)c7c65)c2)c2ccc3oc4ccccc4c3c2)cc1]

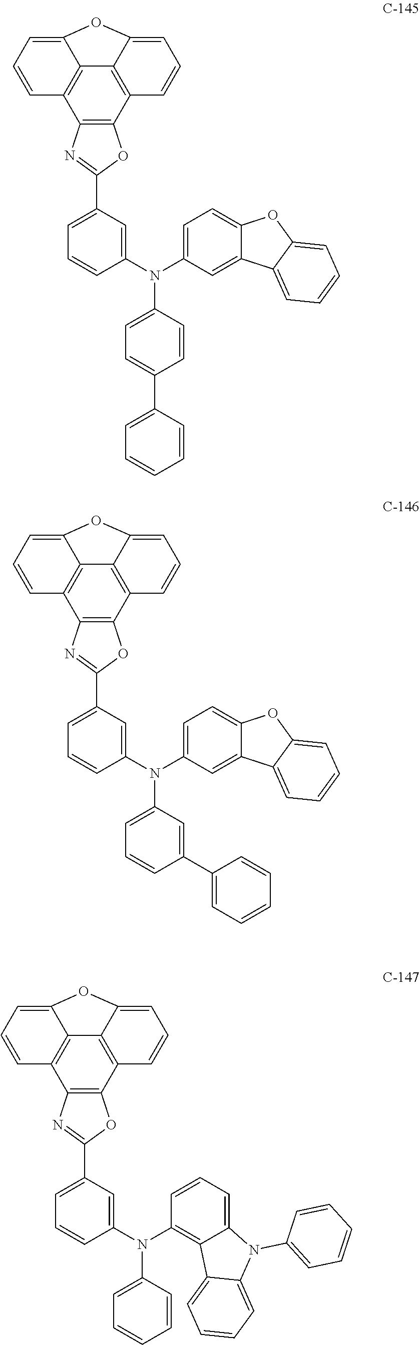 [SMILES: c1ccc(-c2ccc(N(c3cccc(-c4nc5c6cccc7oc8cccc(c5o4)c8c76)c3)c3ccc4oc5ccccc5c4c3)cc2)cc1 ; c1ccc(-c2cccc(N(c3cccc(-c4nc5c6cccc7oc8cccc(c5o4)c8c76)c3)c3ccc4oc5ccccc5c4c3)c2)cc1 ; c1ccc(N(c2cccc(-c3nc4c5cccc6oc7cccc(c4o3)c7c65)c2)c2cccc3c2c2ccccc2n3-c2ccccc2)cc1]

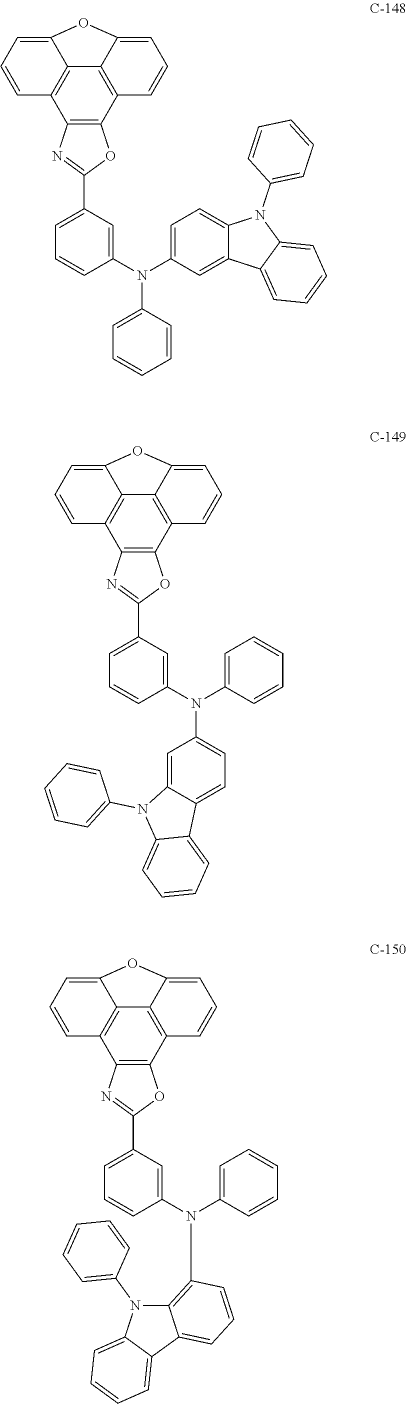 [SMILES: c1ccc(N(c2cccc(-c3nc4c5cccc6oc7cccc(c4o3)c7c65)c2)c2ccc3c(c2)c2ccccc2n3-c2ccccc2)cc1 ; c1ccc(N(c2cccc(-c3nc4c5cccc6oc7cccc(c4o3)c7c65)c2)c2ccc3c4ccccc4n(-c4ccccc4)c3c2)cc1 ; c1ccc(N(c2cccc(-c3nc4c5cccc6oc7cccc(c4o3)c7c65)c2)c2cccc3c4ccccc4n(-c4ccccc4)c23)cc1]

The present disclosure provides an organic electroluminescent material comprising the organic electroluminescent compound represented by formula 1, and an organic electroluminescent device comprising the same.

The organic electroluminescent material may consist of the organic electroluminescent compound of the present disclosure alone, and may further comprise conventional materials included in an organic electroluminescent material.

The organic electroluminescent compound represented by formula 1 may comprised in at least one layer selected from the group consisting of a light-emitting layer, a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer, and preferably in at least one layer of a light-emitting layer (host material), a hole transport zone, and an electron transport zone.

In addition, the present disclosure provides a plurality of host materials comprising at least one first host material comprising an organic electroluminescent compound according to claim 1 and at least one second host material different from the first host material. According to one embodiment of the present disclosure, the second host material may comprise an organic electroluminescent compound represented by the following formula 2.

In formula 2, X₁₁ to X₁₃ each independently represent N, CH, or CD. According to one embodiment of the present disclosure, X₁₁ to X₁₃ each independently represent N or CH. For example, X₁₁ to X₁₃ each independently represent N.

In formula 2, L₂₁ to L₂₃ each independently represent a single bond, a substituted or unsubstituted (C6-C30)arylene, or a substituted or unsubstituted (3- to 30-membered)heteroarylene. According to one embodiment of the present disclosure, L₂₁ to L₂₃ each independently represent a single bond, a substituted or unsubstituted (C6-C25)arylene, or a substituted or unsubstituted (3- to 25-membered) heteroarylene. According to another embodiment of the present disclosure, L₂₁ to L₂₃ each independently represent a single bond, a substituted or unsubstituted (C6-C20)arylene, or a substituted or unsubstituted (3- to 20-membered)heteroarylene. For example, L₂₁ to L₂₃ each independently may be a single bond, a phenylene, a biphenylene, a naphthylene, or a dibenzofuranylene.

In formula 2, Ar₂₁ to Ar₂₃ each independently represent a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s). According to one embodiment of the present disclosure, Ar₂₁ to Ar₂₃ each independently represent a substituted or unsubstituted (C6-C25)aryl, a substituted or unsubstituted (3- to 25-membered)heteroaryl, a substituted or unsubstituted (C3-C30)cycloalkyl, a substituted or unsubstituted (C1-C25)alkoxy, a substituted or unsubstituted tri(C1-C30)alkylsilyl, or a substituted or unsubstituted fused ring group of a (C3-C30) aliphatic ring(s) and a (C6-C30) aromatic ring(s). According to another embodiment of the present disclosure, Ar₂₁ to Ar₂₃ each independently represent a substituted or unsubstituted (C6-C25)aryl, a substituted or unsubstituted (3- to 25-membered)heteroaryl, a substituted or unsubstituted (C3-C25)cycloalkyl, a substituted or unsubstituted (C1-C20)alkoxy, or a substituted or unsubstituted fused ring group of a (C3-C25) aliphatic ring(s) and a (C6-C25) aromatic ring(s). According to another embodiment of the present disclosure, Ar₂₁ to Ar₂₃ each independently represent a substituted or unsubstituted phenylnaphthyl, a substituted or unsubstituted naphthylphenyl, a substituted or unsubstituted phenyl, a substituted or unsubstituted biphenyl, a substituted or unsubstituted terphenyl, a substituted or unsubstituted triphenylenyl, a substituted or unsubstituted dimethylbenzofluorenyl, a substituted or unsubstituted dimethylfluorenyl, a substituted or unsubstituted phenanthrenyl, a substituted or unsubstituted benzonaphthofuranyl, a substituted or unsubstituted chrysenyl, a substituted or unsubstituted diphenylbenzofluorenyl, a substituted or unsubstituted binaphthyl, a substituted or unsubstituted dibenzofuranyl, a substituted or unsubstituted carbazolyl, a substituted or unsubstituted benzocarbazolyl, or a substituted or unsubstituted dibenzothiophenyl. For example, Ar₂₁ to Ar₂₃ each independently may be a phenyl unsubstituted or substituted with a naphthyl(s) or a dibenzofuranyl(s), a biphenyl, a terphenyl, a triphenyleneyl, a dibenzofuranyl unsubstituted or substituted with a phenyl(s), a dibenzothiophenyl, a dimethylbenzofluorenyl, a dimethylfluorenyl, a naphthyl unsubstituted or substituted with a phenyl(s), a phenanthrenyl, a benzonaphthofuranyl unsubstituted or substituted with a naphthyl(s), a chrysenyl, or a diphenylbenzofluorenyl.

The compound represented by formula 2 may be selected from the group consisting of the following compounds, but is not limited thereto.

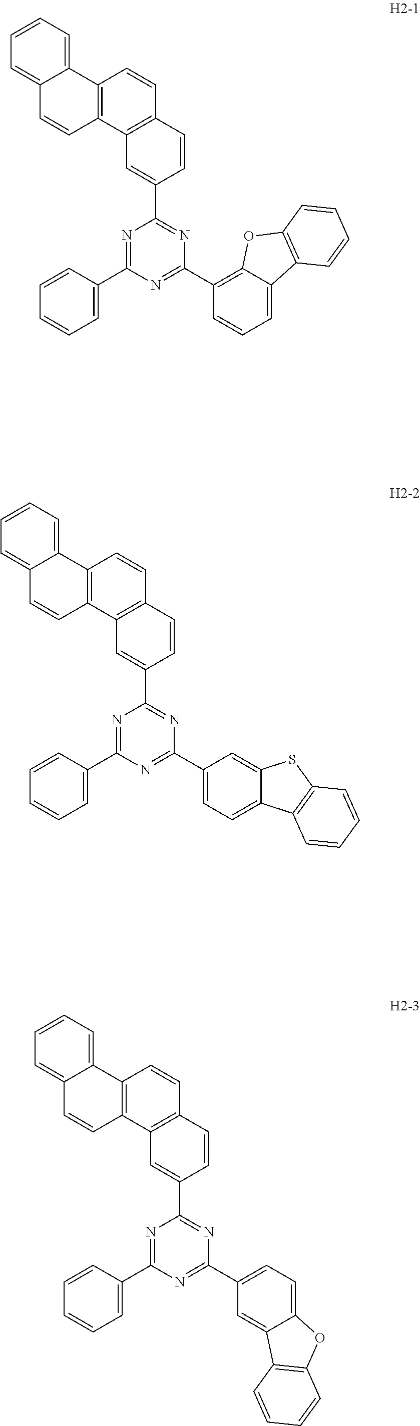 [SMILES: c1ccc(-c2nc(-c3ccc4ccc5c6ccccc6ccc5c4c3)nc(-c3cccc4c3oc3ccccc34)n2)cc1 ; c1ccc(-c2nc(-c3ccc4c(c3)sc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3ccc4oc5ccccc5c4c3)n2)cc1]

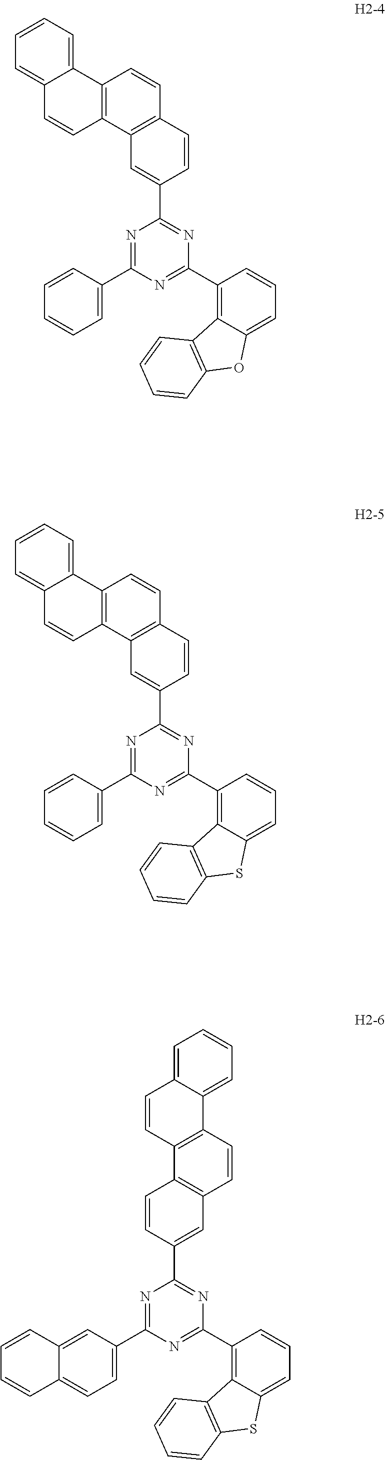 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1 ; c1ccc2cc(-c3nc(-c4ccc5c(ccc6c7ccccc7ccc56)c4)nc(-c4cccc5sc6ccccc6c45)n3)ccc2c1]

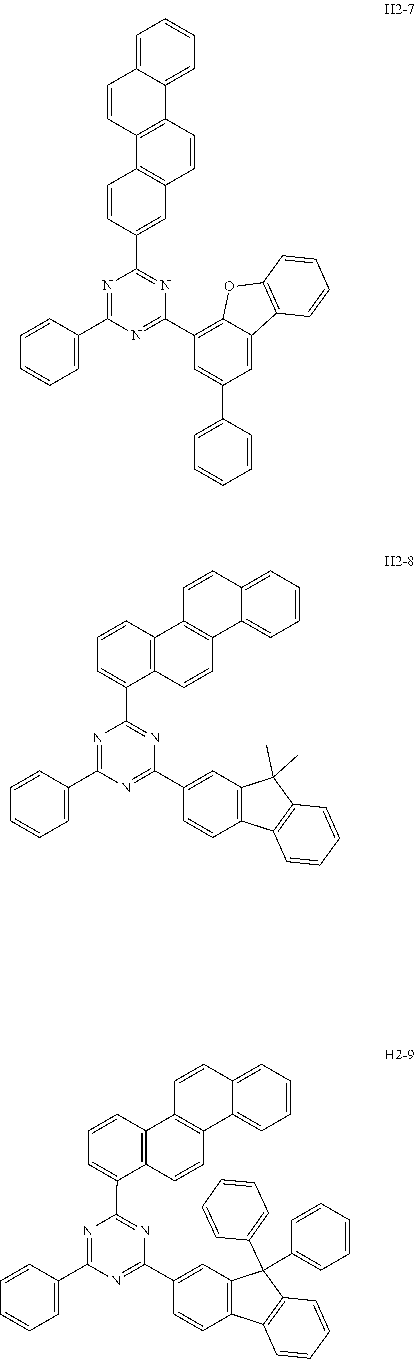 [SMILES: c1ccc(-c2cc(-c3nc(-c4ccccc4)nc(-c4ccc5c(ccc6c7ccccc7ccc56)c4)n3)c3oc4ccccc4c3c2)cc1 ; CC1(C)c2ccccc2-c2ccc(-c3nc(-c4ccccc4)nc(-c4cccc5c4ccc4c6ccccc6ccc54)n3)cc21 ; c1ccc(-c2nc(-c3ccc4c(c3)C(c3ccccc3)(c3ccccc3)c3ccccc3-4)nc(-c3cccc4c3ccc3c5ccccc5ccc43)n2)cc1]

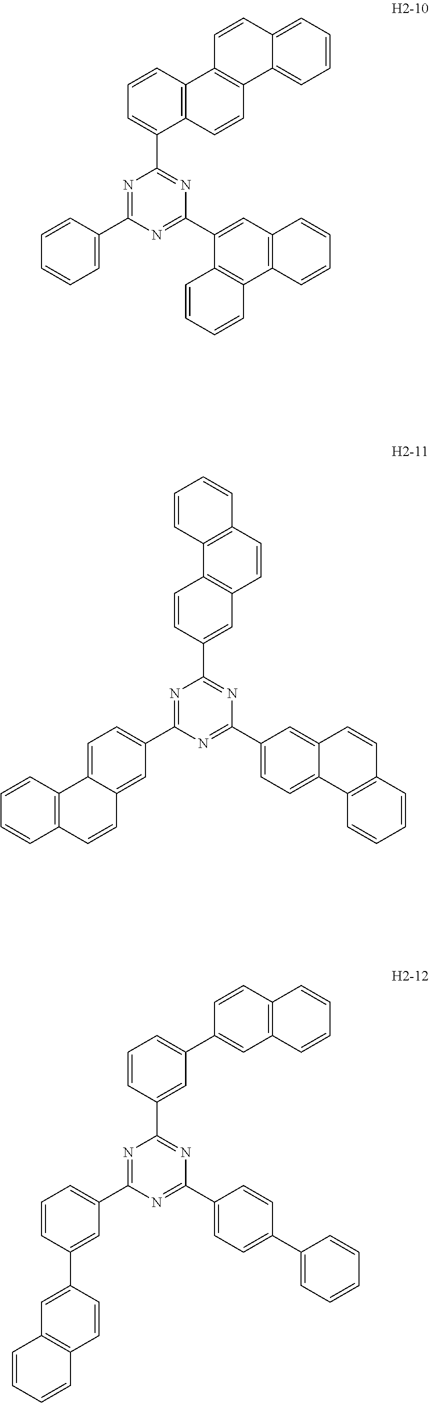 [SMILES: c1ccc(-c2nc(-c3cc4ccccc4c4ccccc34)nc(-c3cccc4c3ccc3c5ccccc5ccc43)n2)cc1 ; c1ccc2c(c1)ccc1cc(-c3nc(-c4ccc5c(ccc6ccccc65)c4)nc(-c4ccc5c(ccc6ccccc65)c4)n3)ccc12 ; c1ccc(-c2ccc(-c3nc(-c4cccc(-c5ccc6ccccc6c5)c4)nc(-c4cccc(-c5ccc6ccccc6c5)c4)n3)cc2)cc1]

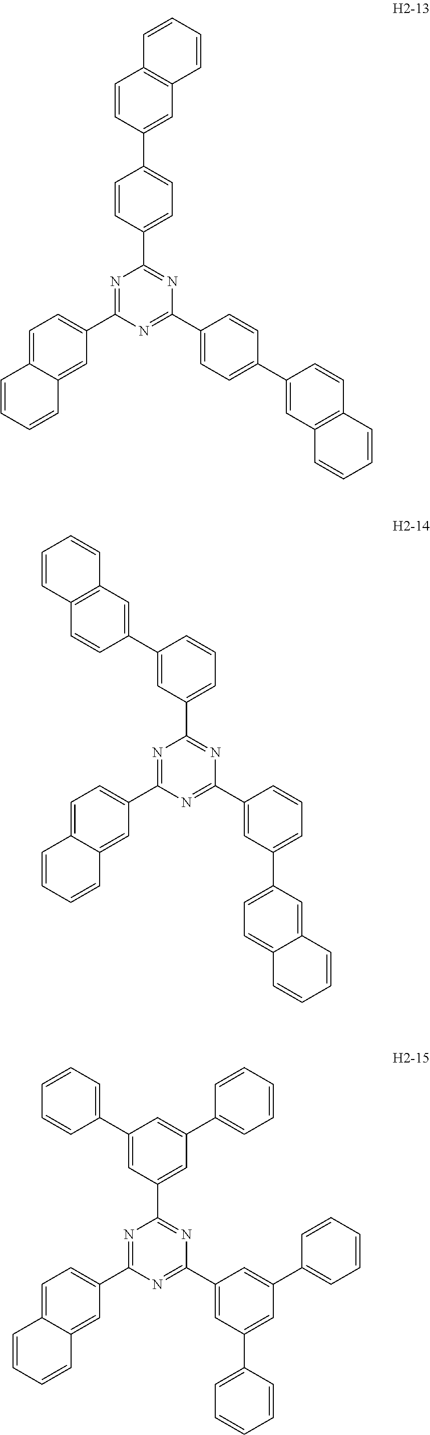 [SMILES: c1ccc2cc(-c3ccc(-c4nc(-c5ccc(-c6ccc7ccccc7c6)cc5)nc(-c5ccc6ccccc6c5)n4)cc3)ccc2c1 ; c1cc(-c2ccc3ccccc3c2)cc(-c2nc(-c3cccc(-c4ccc5ccccc5c4)c3)nc(-c3ccc4ccccc4c3)n2)c1 ; c1ccc(-c2cc(-c3ccccc3)cc(-c3nc(-c4cc(-c5ccccc5)cc(-c5ccccc5)c4)nc(-c4ccc5ccccc5c4)n3)c2)cc1]

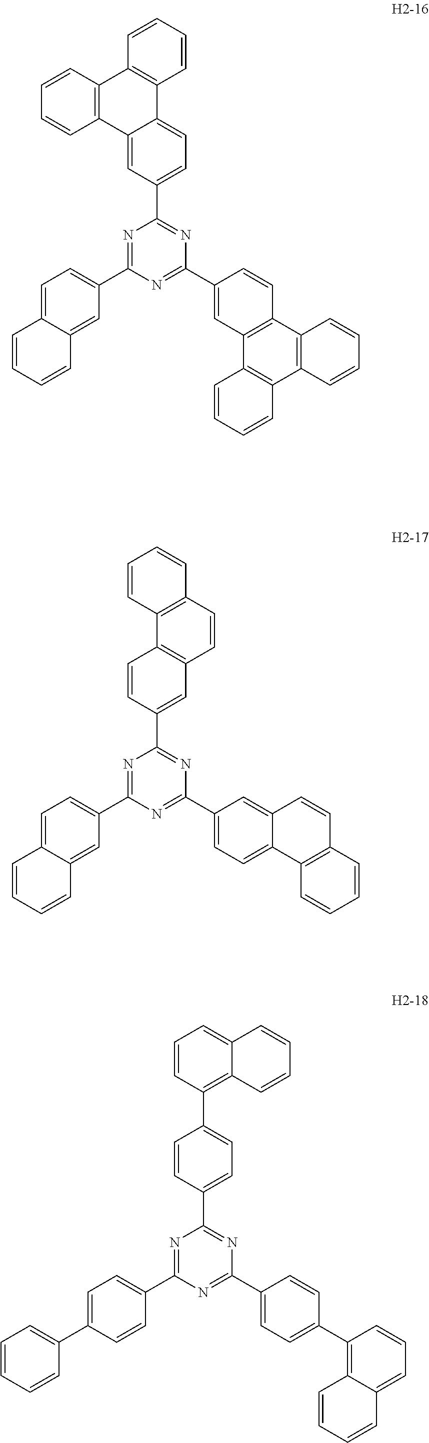 [SMILES: c1ccc2cc(-c3nc(-c4ccc5c6ccccc6c6ccccc6c5c4)nc(-c4ccc5c6ccccc6c6ccccc6c5c4)n3)ccc2c1 ; c1ccc2cc(-c3nc(-c4ccc5c(ccc6ccccc65)c4)nc(-c4ccc5c(ccc6ccccc65)c4)n3)ccc2c1 ; c1ccc(-c2ccc(-c3nc(-c4ccc(-c5cccc6ccccc56)cc4)nc(-c4ccc(-c5cccc6ccccc56)cc4)n3)cc2)cc1]

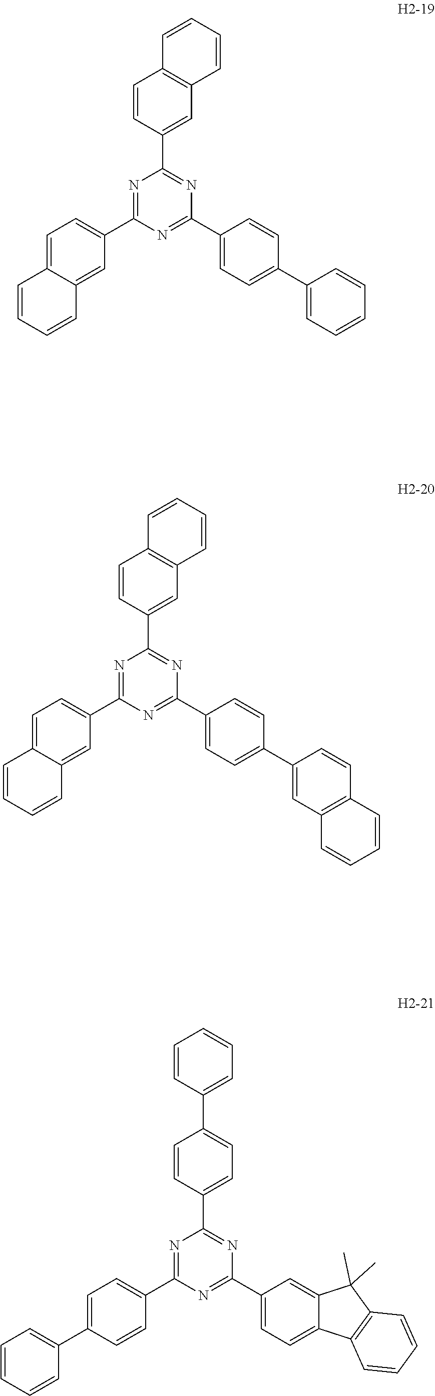 [SMILES: c1ccc(-c2ccc(-c3nc(-c4ccc5ccccc5c4)nc(-c4ccc5ccccc5c4)n3)cc2)cc1 ; c1ccc2cc(-c3ccc(-c4nc(-c5ccc6ccccc6c5)nc(-c5ccc6ccccc6c5)n4)cc3)ccc2c1 ; CC1(C)c2ccccc2-c2ccc(-c3nc(-c4ccc(-c5ccccc5)cc4)nc(-c4ccc(-c5ccccc5)cc4)n3)cc21]

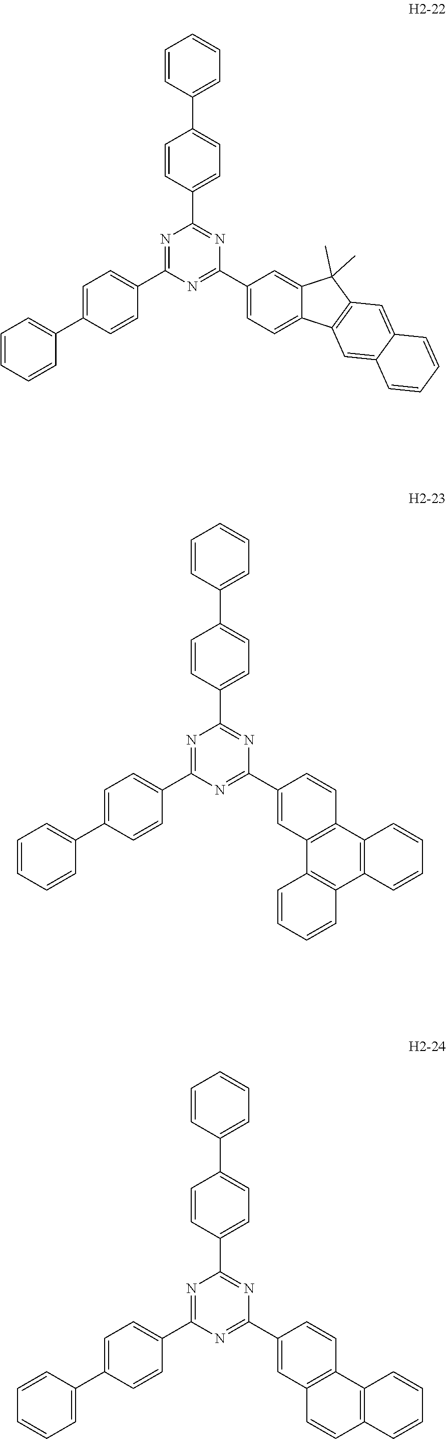

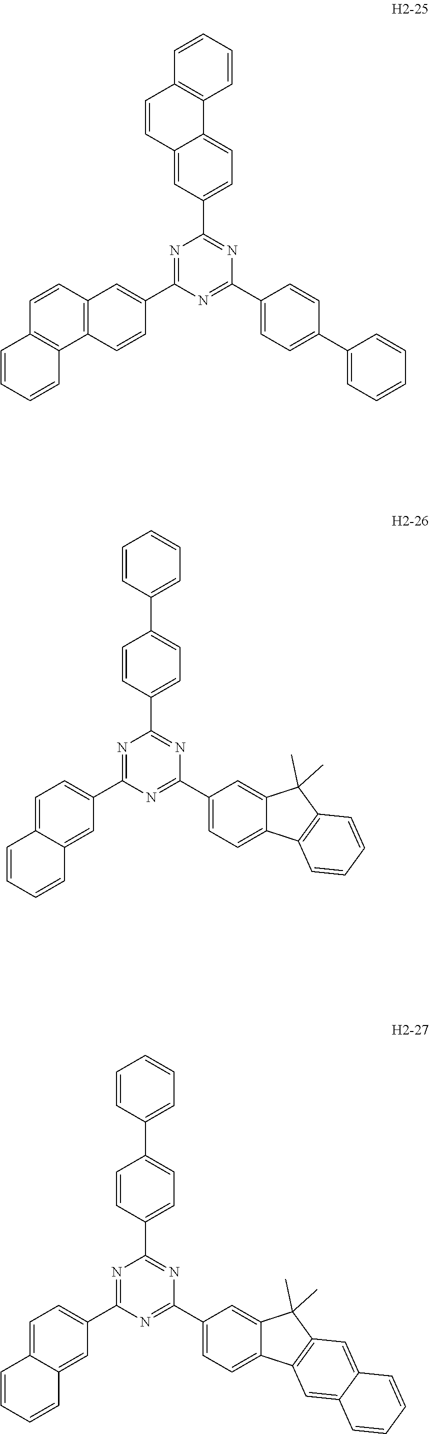 [SMILES: c1ccc(-c2ccc(-c3nc(-c4ccc5c(ccc6ccccc65)c4)nc(-c4ccc5c(ccc6ccccc65)c4)n3)cc2)cc1 ; CC1(C)c2ccccc2-c2ccc(-c3nc(-c4ccc(-c5ccccc5)cc4)nc(-c4ccc5ccccc5c4)n3)cc21 ; CC1(C)c2cc(-c3nc(-c4ccc(-c5ccccc5)cc4)nc(-c4ccc5ccccc5c4)n3)ccc2-c2cc3ccccc3cc21]

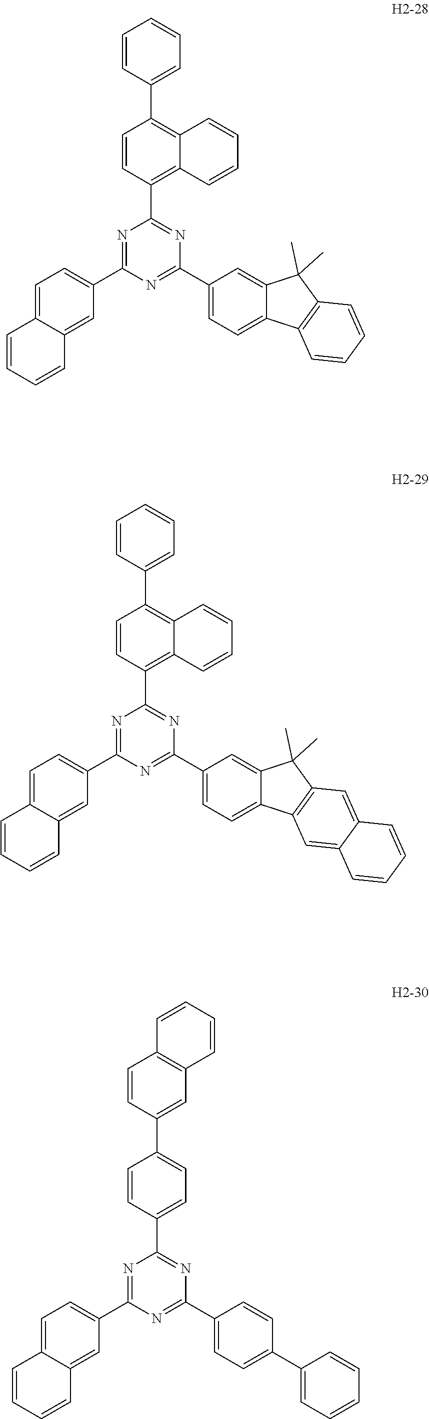 [SMILES: CC1(C)c2ccccc2-c2ccc(-c3nc(-c4ccc5ccccc5c4)nc(-c4ccc(-c5ccccc5)c5ccccc45)n3)cc21 ; CC1(C)c2cc(-c3nc(-c4ccc5ccccc5c4)nc(-c4ccc(-c5ccccc5)c5ccccc45)n3)ccc2-c2cc3ccccc3cc21 ; c1ccc(-c2ccc(-c3nc(-c4ccc(-c5ccc6ccccc6c5)cc4)nc(-c4ccc5ccccc5c4)n3)cc2)cc1]

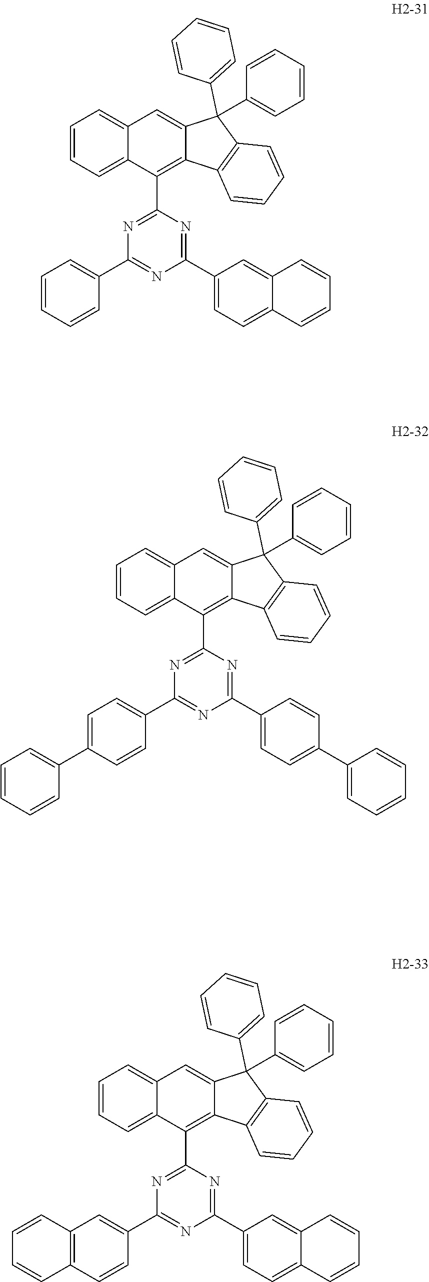 [SMILES: c1ccc(-c2nc(-c3ccc4ccccc4c3)nc(-c3c4c(cc5ccccc35)C(c3ccccc3)(c3ccccc3)c3ccccc3-4)n2)cc1 ; c1ccc(-c2ccc(-c3nc(-c4ccc(-c5ccccc5)cc4)nc(-c4c5c(cc6ccccc46)C(c4ccccc4)(c4ccccc4)c4ccccc4-5)n3)cc2)cc1 ; c1ccc(C2(c3ccccc3)c3ccccc3-c3c2cc2ccccc2c3-c2nc(-c3ccc4ccccc4c3)nc(-c3ccc4ccccc4c3)n2)cc1]

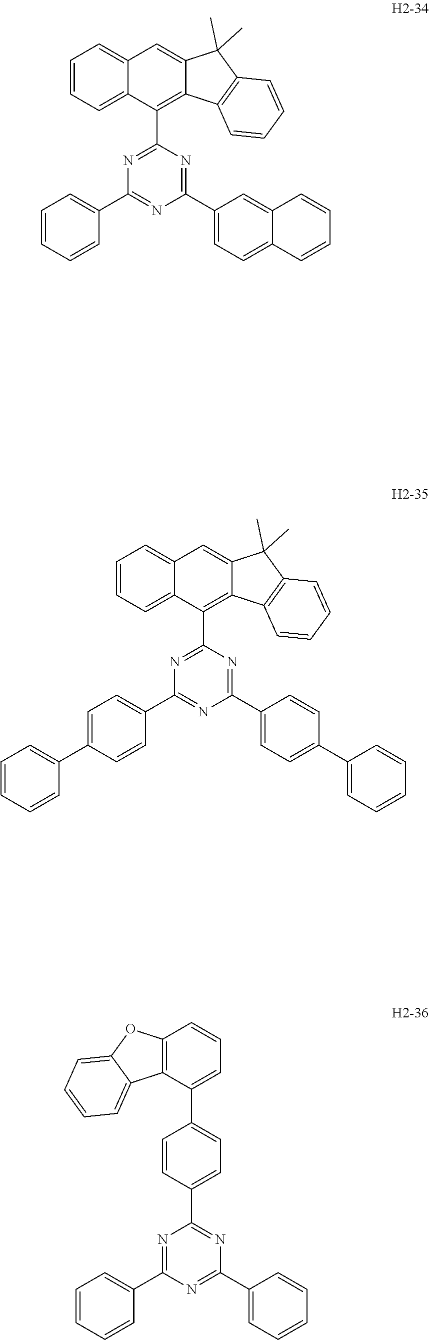 [SMILES: CC1(C)c2ccccc2-c2c1cc1ccccc1c2-c1nc(-c2ccccc2)nc(-c2ccc3ccccc3c2)n1 ; CC1(C)c2ccccc2-c2c1cc1ccccc1c2-c1nc(-c2ccc(-c3ccccc3)cc2)nc(-c2ccc(-c3ccccc3)cc2)n1 ; c1ccc(-c2nc(-c3ccccc3)nc(-c3ccc(-c4cccc5oc6ccccc6c45)cc3)n2)cc1]

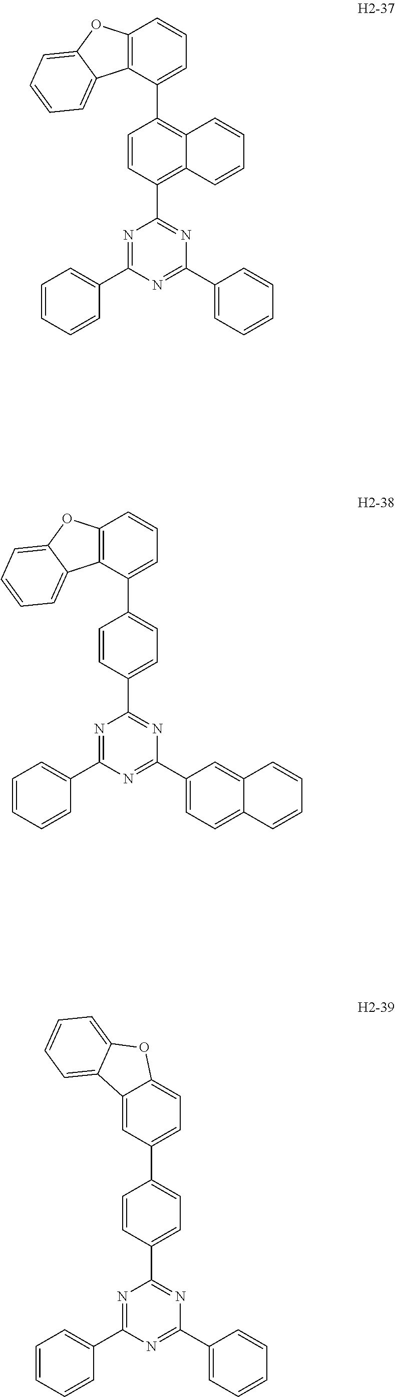 [SMILES: c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1 ; c1ccc(-c2nc(-c3ccccc3)nc(-c3ccc(-c4ccc5oc6ccccc6c5c4)cc3)n2)cc1]

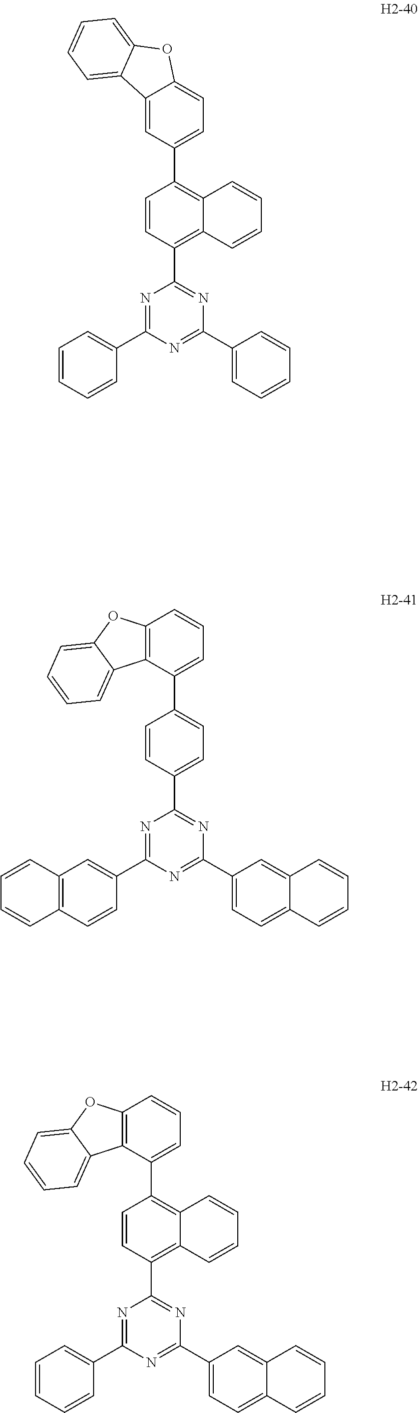 [SMILES: c1ccc(-c2nc(-c3ccccc3)nc(-c3ccc(-c4ccc5oc6ccccc6c5c4)c4ccccc34)n2)cc1 ; c1ccc2cc(-c3nc(-c4ccc(-c5cccc6oc7ccccc7c56)cc4)nc(-c4ccc5ccccc5c4)n3)ccc2c1 ; c1ccc(-c2nc(-c3ccc4ccccc4c3)nc(-c3ccc(-c4cccc5oc6ccccc6c45)c4ccccc34)n2)cc1]

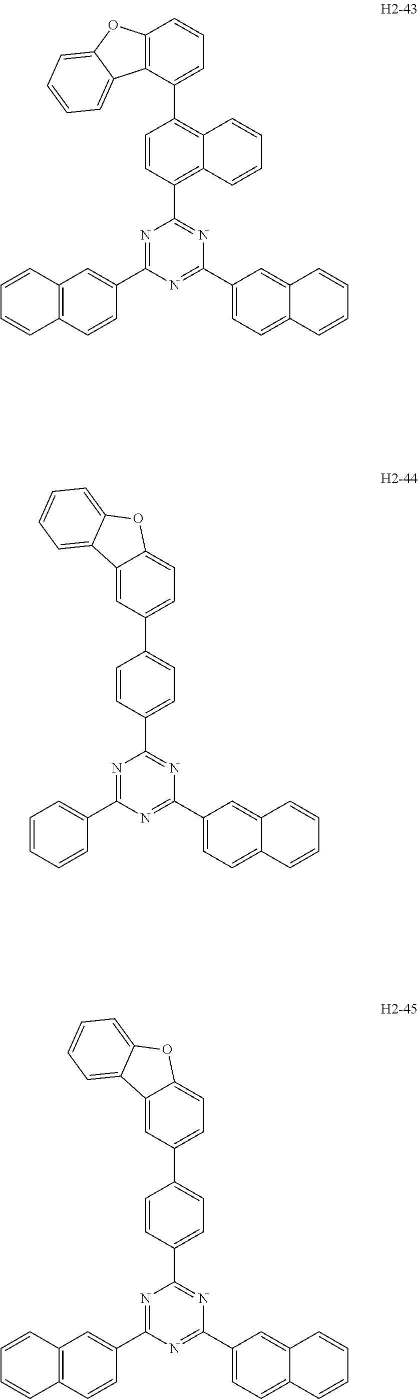 [SMILES: c1ccc2cc(-c3nc(-c4ccc5ccccc5c4)nc(-c4ccc(-c5cccc6oc7ccccc7c56)c5ccccc45)n3)ccc2c1 ; c1ccc(-c2nc(-c3ccc(-c4ccc5oc6ccccc6c5c4)cc3)nc(-c3ccc4ccccc4c3)n2)cc1 ; c1ccc2cc(-c3nc(-c4ccc(-c5ccc6oc7ccccc7c6c5)cc4)nc(-c4ccc5ccccc5c4)n3)ccc2c1]

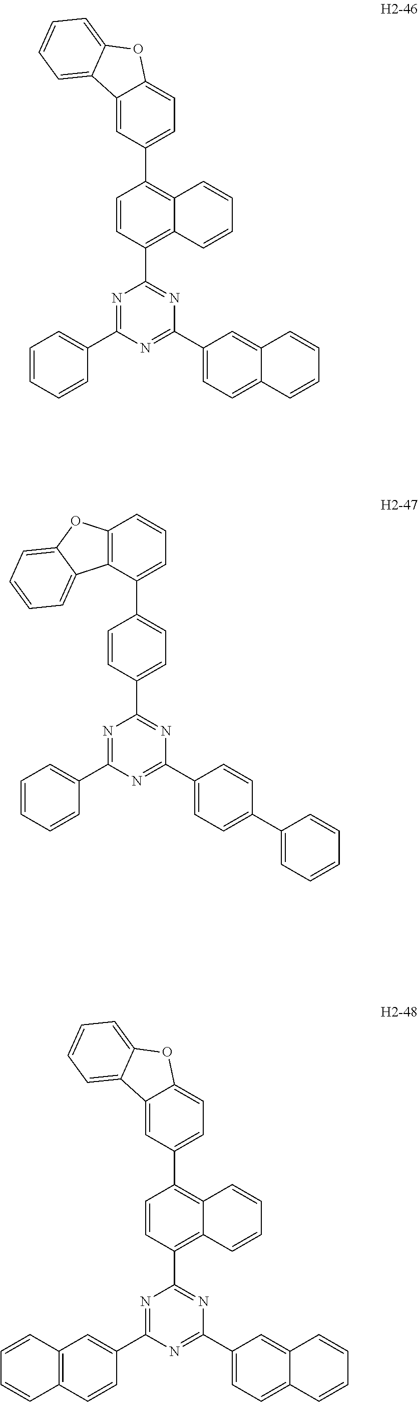 [SMILES: c1ccc(-c2nc(-c3ccc4ccccc4c3)nc(-c3ccc(-c4ccc5oc6ccccc6c5c4)c4ccccc34)n2)cc1 ; c1ccc(-c2ccc(-c3nc(-c4ccccc4)nc(-c4ccc(-c5cccc6oc7ccccc7c56)cc4)n3)cc2)cc1 ; c1ccc2cc(-c3nc(-c4ccc5ccccc5c4)nc(-c4ccc(-c5ccc6oc7ccccc7c6c5)c5ccccc45)n3)ccc2c1]

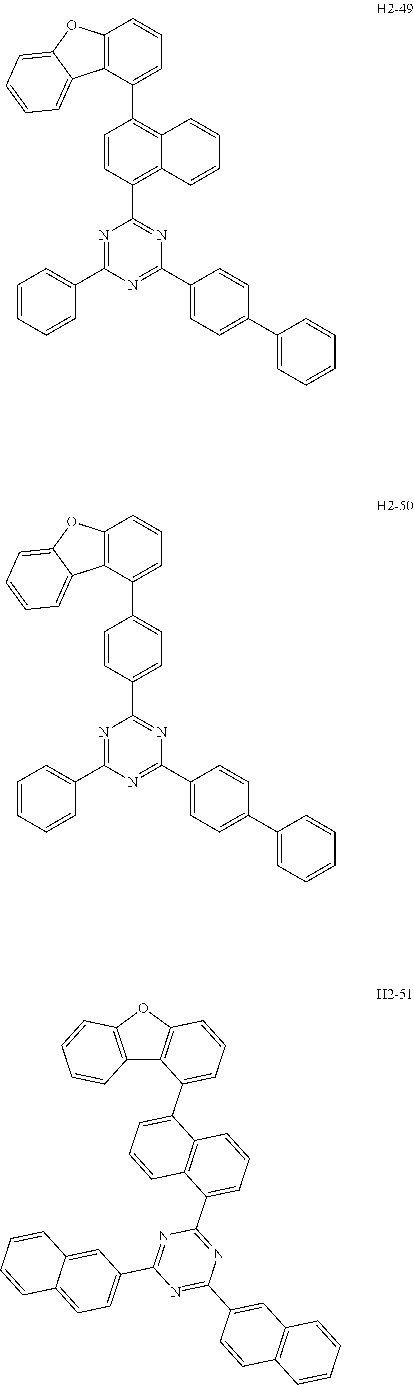 [SMILES: c1ccc(-c2ccc(-c3nc(-c4ccccc4)nc(-c4ccc(-c5cccc6oc7ccccc7c56)c5ccccc45)n3)cc2)cc1 ; c1ccc(-c2ccc(-c3nc(-c4ccccc4)nc(-c4ccc(-c5cccc6oc7ccccc7c56)cc4)n3)cc2)cc1 ; c1ccc2cc(-c3nc(-c4ccc5ccccc5c4)nc(-c4cccc5c(-c6cccc7oc8ccccc8c67)cccc45)n3)ccc2c1]

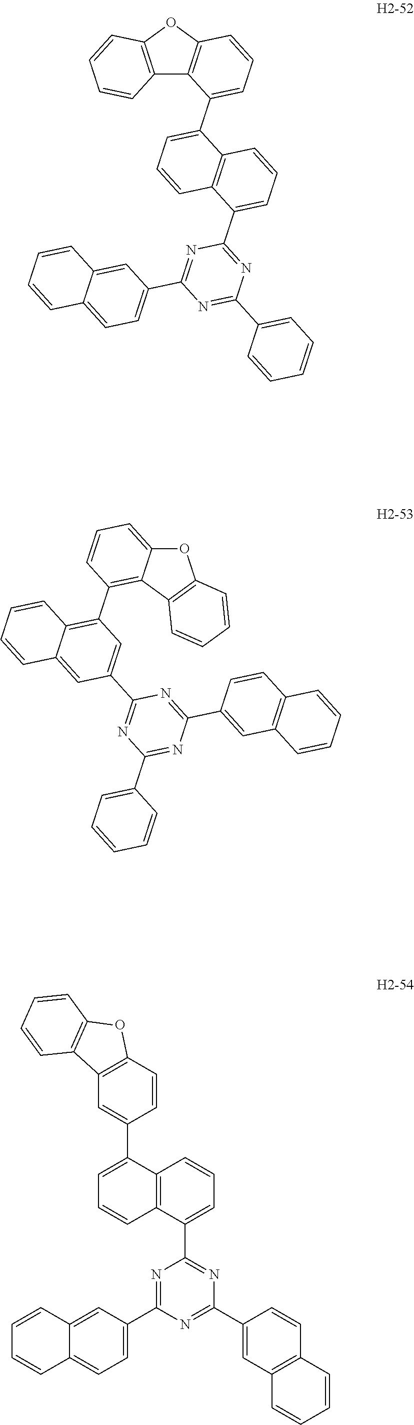 [SMILES: c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1 ; c1ccc2cc(-c3nc(-c4ccc5ccccc5c4)nc(-c4cccc5c(-c6ccc7oc8ccccc8c7c6)cccc45)n3)ccc2c1]

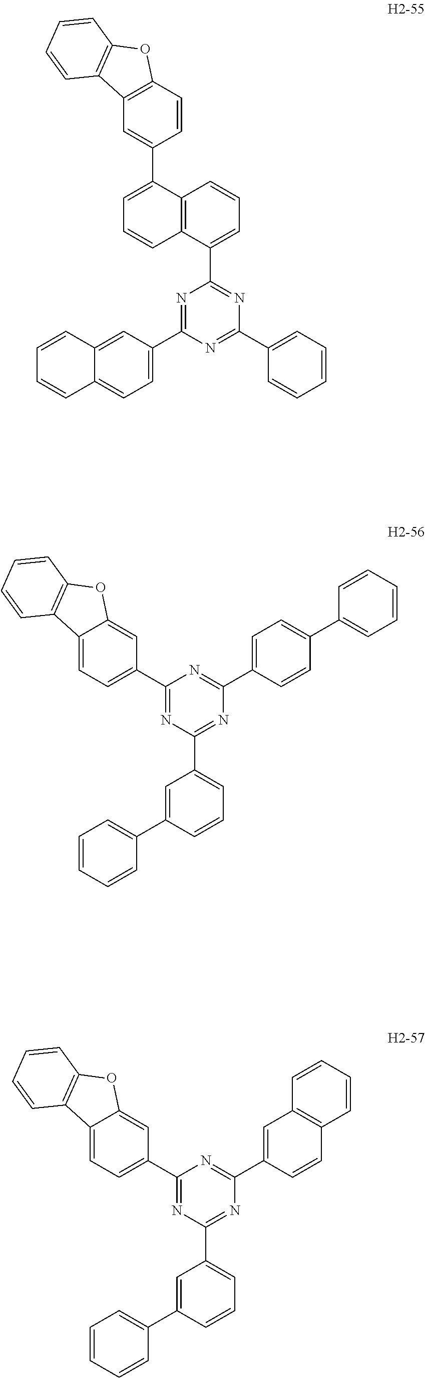 [SMILES: c1ccc(-c2nc(-c3ccc4ccccc4c3)nc(-c3cccc4c(-c5ccc6oc7ccccc7c6c5)cccc34)n2)cc1 ; c1ccc(-c2ccc(-c3nc(-c4cccc(-c5ccccc5)c4)nc(-c4ccc5c(c4)oc4ccccc45)n3)cc2)cc1 ; c1ccc(-c2cccc(-c3nc(-c4ccc5ccccc5c4)nc(-c4ccc5c(c4)oc4ccccc45)n3)c2)cc1]

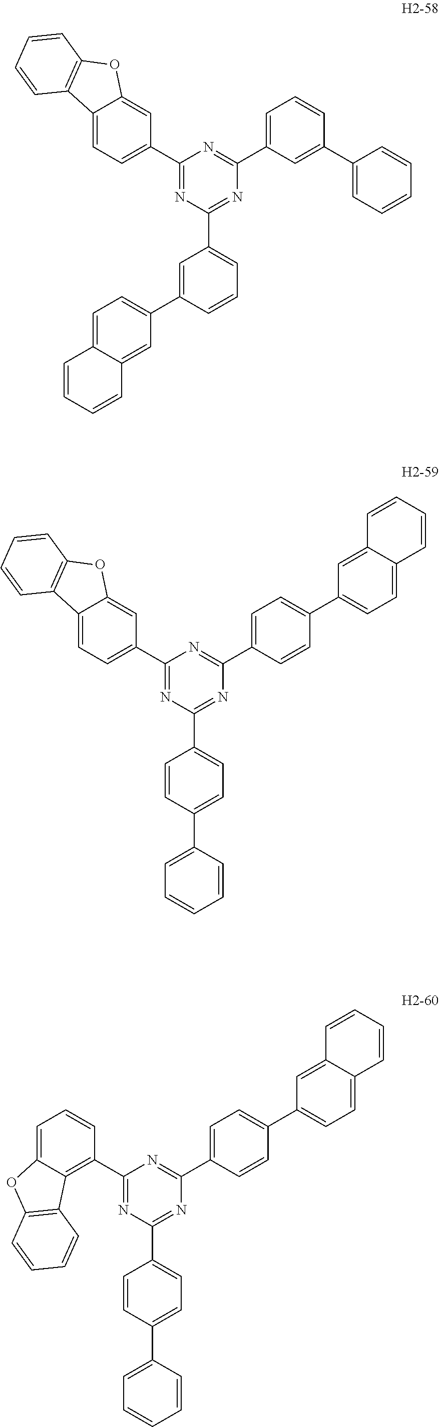 [SMILES: c1ccc(-c2cccc(-c3nc(-c4cccc(-c5ccc6ccccc6c5)c4)nc(-c4ccc5c(c4)oc4ccccc45)n3)c2)cc1 ; c1ccc(-c2ccc(-c3nc(-c4ccc(-c5ccc6ccccc6c5)cc4)nc(-c4ccc5c(c4)oc4ccccc45)n3)cc2)cc1 ; c1ccc(-c2ccc(-c3nc(-c4ccc(-c5ccc6ccccc6c5)cc4)nc(-c4cccc5oc6ccccc6c45)n3)cc2)cc1]

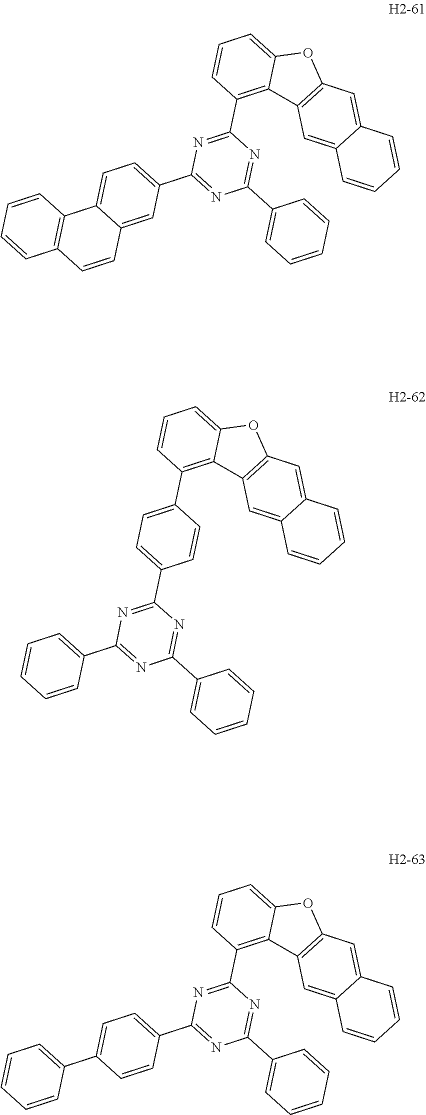 [SMILES: c1ccc(-c2nc(-c3ccc4c(ccc5ccccc54)c3)nc(-c3cccc4oc5cc6ccccc6cc5c34)n2)cc1 ; c1ccc(-c2nc(-c3ccccc3)nc(-c3ccc(-c4cccc5oc6cc7ccccc7cc6c45)cc3)n2)cc1 ; c1ccc(-c2ccc(-c3nc(-c4ccccc4)nc(-c4cccc5oc6cc7ccccc7cc6c45)n3)cc2)cc1]

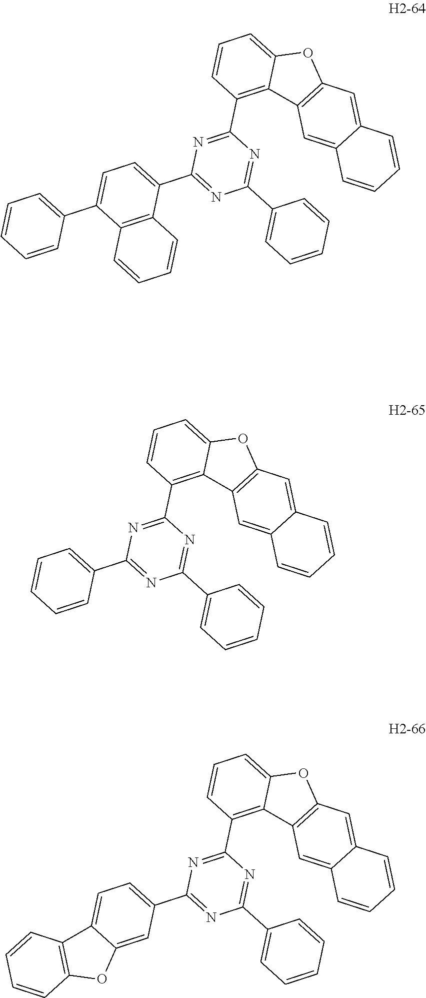 [SMILES: c1ccc(-c2nc(-c3ccc(-c4ccccc4)c4ccccc34)nc(-c3cccc4oc5cc6ccccc6cc5c34)n2)cc1 ; c1ccc(-c2nc(-c3ccccc3)nc(-c3cccc4oc5cc6ccccc6cc5c34)n2)cc1 ; c1ccc(-c2nc(-c3ccc4c(c3)oc3ccccc34)nc(-c3cccc4oc5cc6ccccc6cc5c34)n2)cc1]

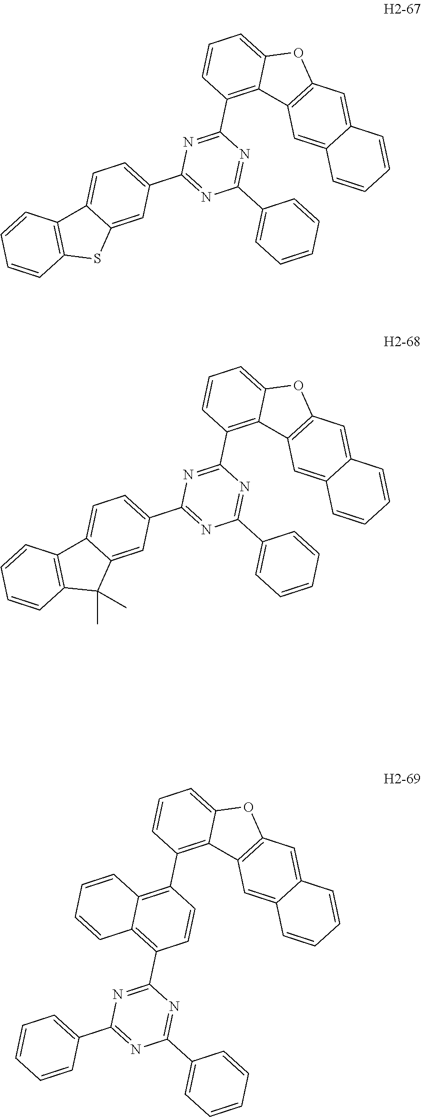 [SMILES: c1ccc(-c2nc(-c3ccc4c(c3)sc3ccccc34)nc(-c3cccc4oc5cc6ccccc6cc5c34)n2)cc1 ; CC1(C)c2ccccc2-c2ccc(-c3nc(-c4ccccc4)nc(-c4cccc5oc6cc7ccccc7cc6c45)n3)cc21 ; c1ccc(-c2nc(-c3ccccc3)nc(-c3ccc(-c4cccc5oc6cc7ccccc7cc6c45)c4ccccc34)n2)cc1]

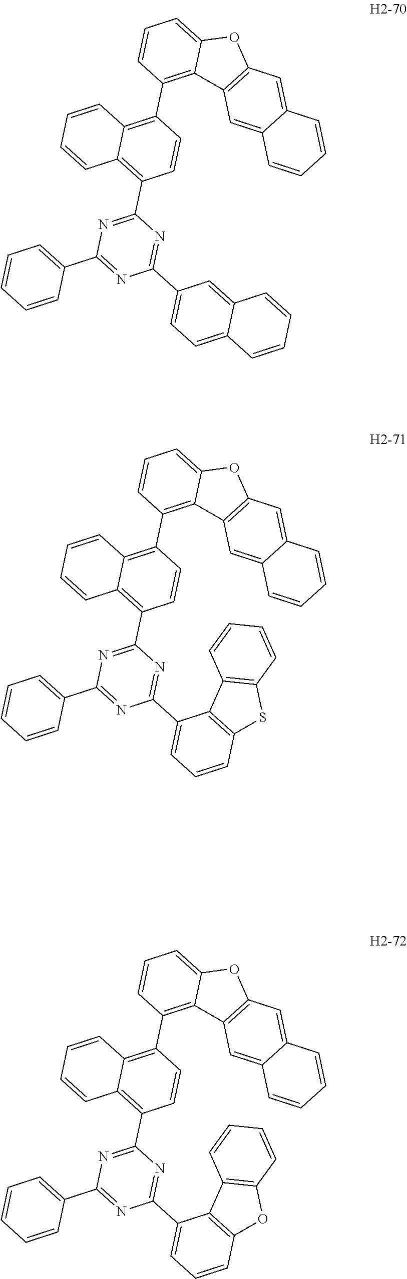 [SMILES: c1ccc(-c2nc(-c3ccc4ccccc4c3)nc(-c3ccc(-c4cccc5oc6cc7ccccc7cc6c45)c4ccccc34)n2)cc1 ; c1ccc(-c2nc(-c3ccc(-c4cccc5oc6cc7ccccc7cc6c45)c4ccccc34)nc(-c3cccc4sc5ccccc5c34)n2)cc1 ; c1ccc(-c2nc(-c3ccc(-c4cccc5oc6cc7ccccc7cc6c45)c4ccccc34)nc(-c3cccc4oc5ccccc5c34)n2)cc1]

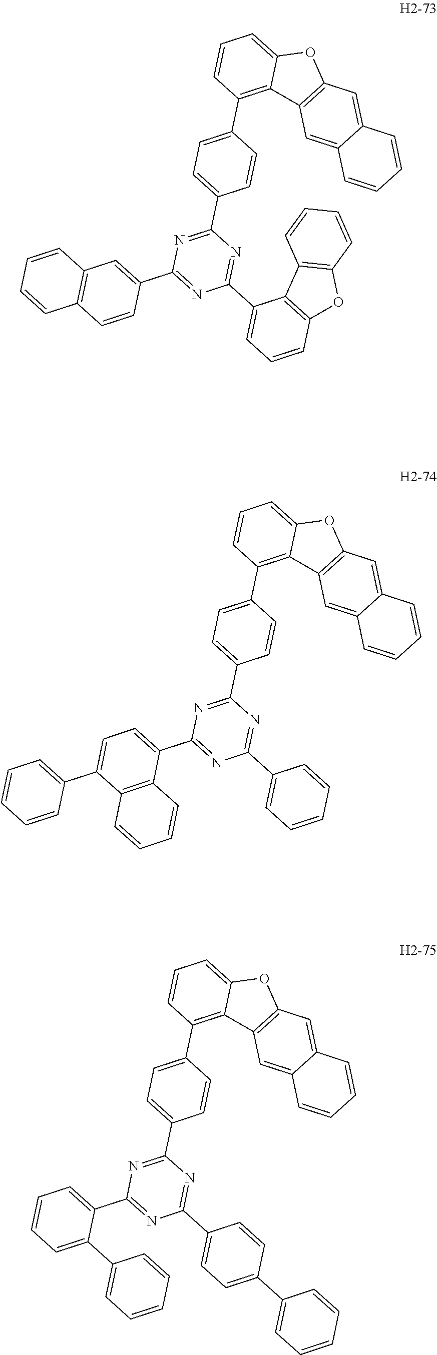 [SMILES: c1ccc2cc(-c3nc(-c4ccc(-c5cccc6oc7cc8ccccc8cc7c56)cc4)nc(-c4cccc5oc6ccccc6c45)n3)ccc2c1 ; c1ccc(-c2nc(-c3ccc(-c4cccc5oc6cc7ccccc7cc6c45)cc3)nc(-c3ccc(-c4ccccc4)c4ccccc34)n2)cc1 ; c1ccc(-c2ccc(-c3nc(-c4ccc(-c5cccc6oc7cc8ccccc8cc7c56)cc4)nc(-c4ccccc4-c4ccccc4)n3)cc2)cc1]

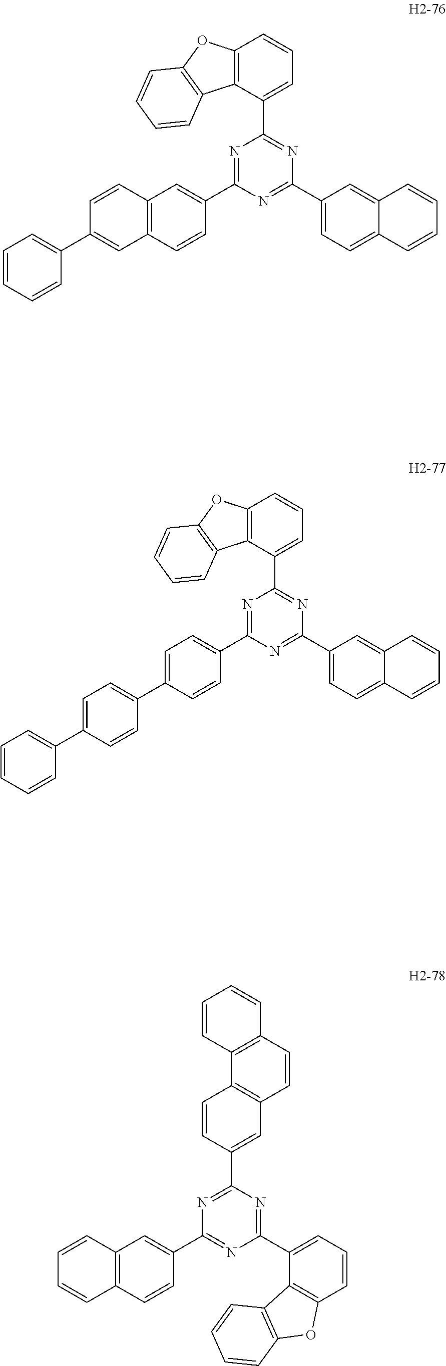 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccc6ccccc6c5)nc(-c5cccc6oc7ccccc7c56)n4)cc3)cc2)cc1 ; c1ccc2cc(-c3nc(-c4ccc5c(ccc6ccccc65)c4)nc(-c4cccc5oc6ccccc6c45)n3)ccc2c1]

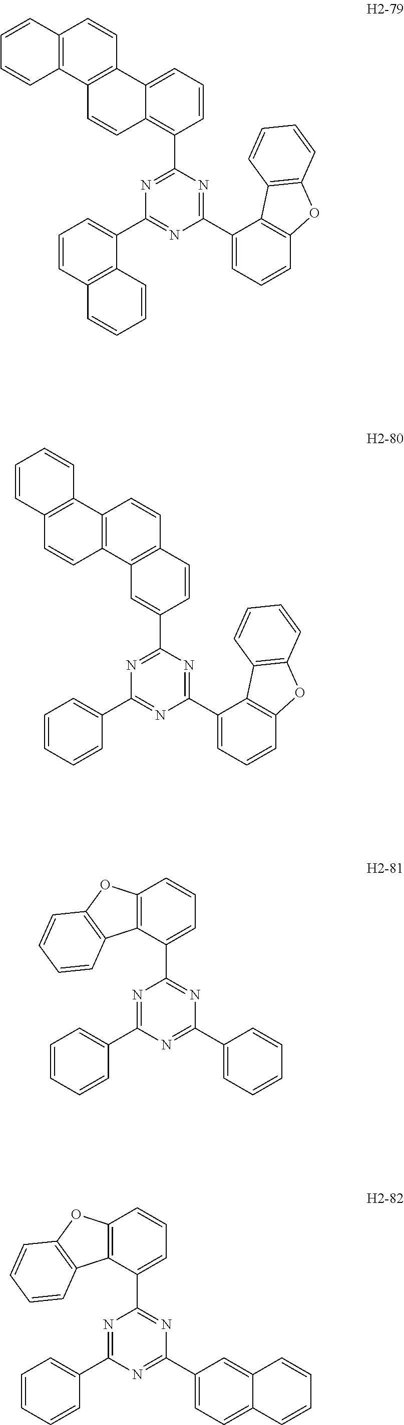 [SMILES: c1ccc2c(-c3nc(-c4cccc5c4ccc4c6ccccc6ccc54)nc(-c4cccc5oc6ccccc6c45)n3)cccc2c1 ; c1ccc(-c2nc(-c3ccc4ccc5c6ccccc6ccc5c4c3)nc(-c3cccc4oc5ccccc5c34)n2)cc1 ; c1ccc(-c2nc(-c3ccccc3)nc(-c3cccc4oc5ccccc5c34)n2)cc1 ; c1ccc(-c2nc(-c3ccc4ccccc4c3)nc(-c3cccc4oc5ccccc5c34)n2)cc1]

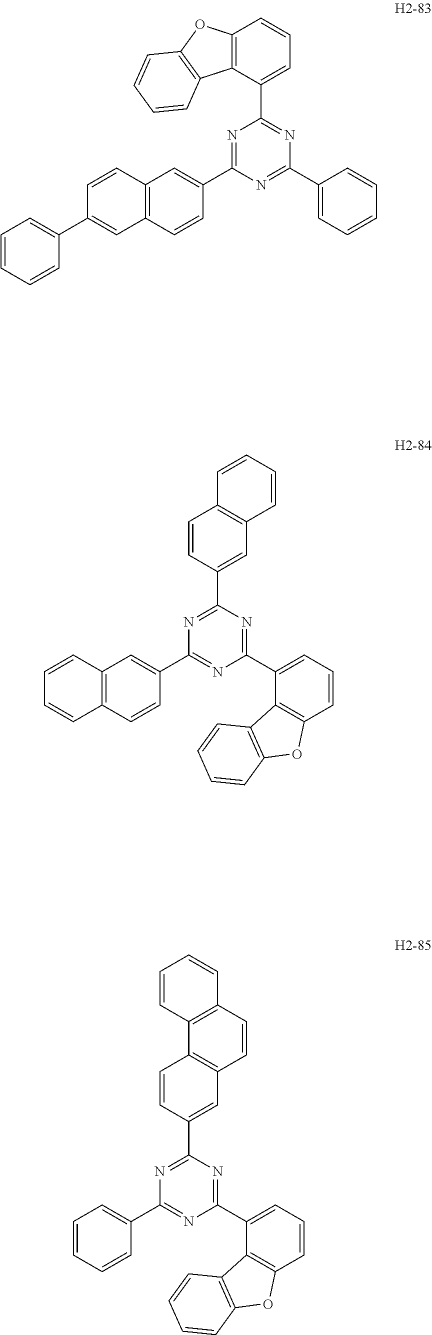 [SMILES: c1ccc(-c2ccc3cc(-c4nc(-c5ccccc5)nc(-c5cccc6oc7ccccc7c56)n4)ccc3c2)cc1 ; c1ccc2cc(-c3nc(-c4ccc5ccccc5c4)nc(-c4cccc5oc6ccccc6c45)n3)ccc2c1 ; c1ccc(-c2nc(-c3ccc4c(ccc5ccccc54)c3)nc(-c3cccc4oc5ccccc5c34)n2)cc1]

The combination of at least one of Compounds C-1 to C-150 and at least one of Compounds H2-1 to H2-85 may be used in an organic electroluminescent device.

The compound represented by formula 1 according to the present disclosure may be produced by referring to the following reaction schemes 1 to 10, but is not limited thereto.

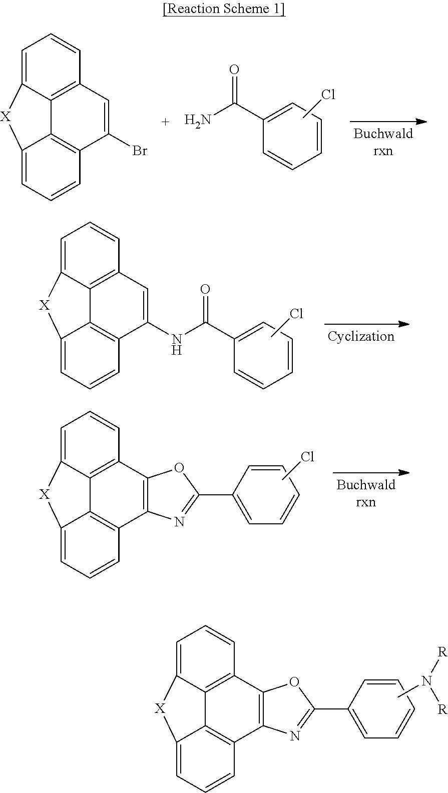

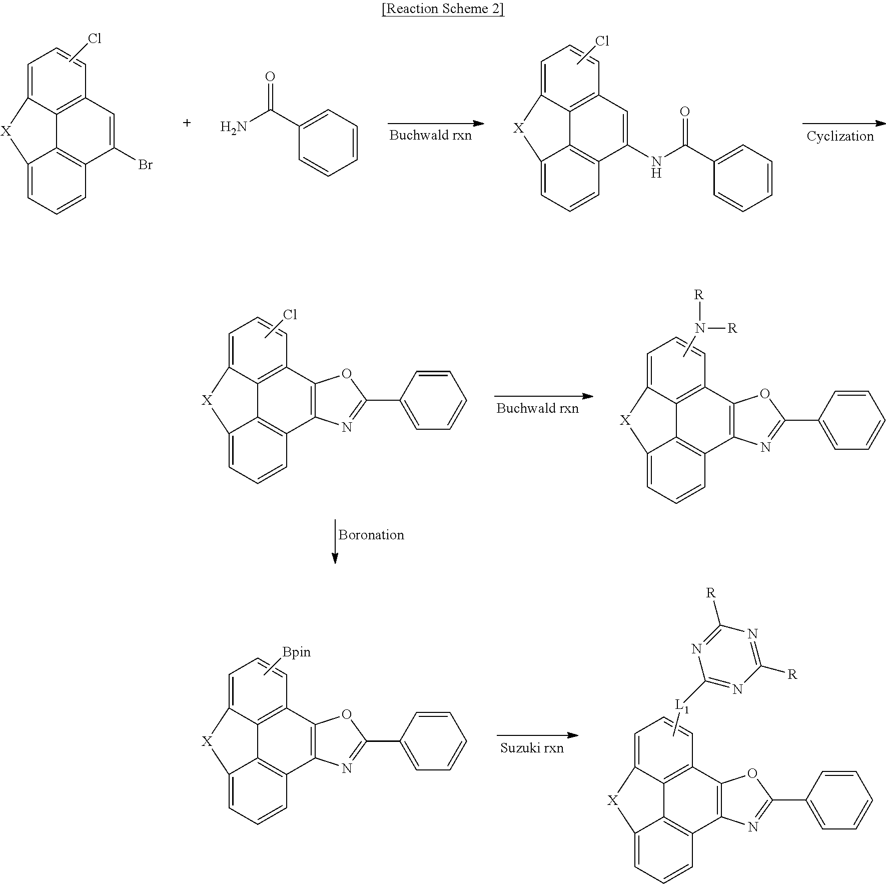

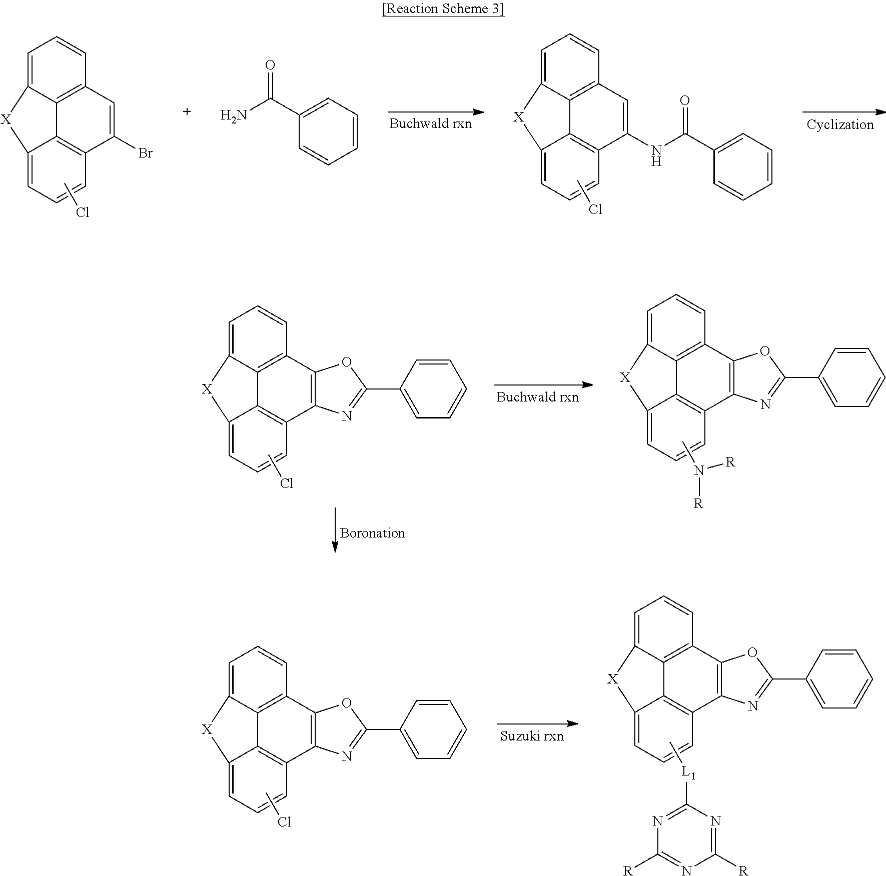

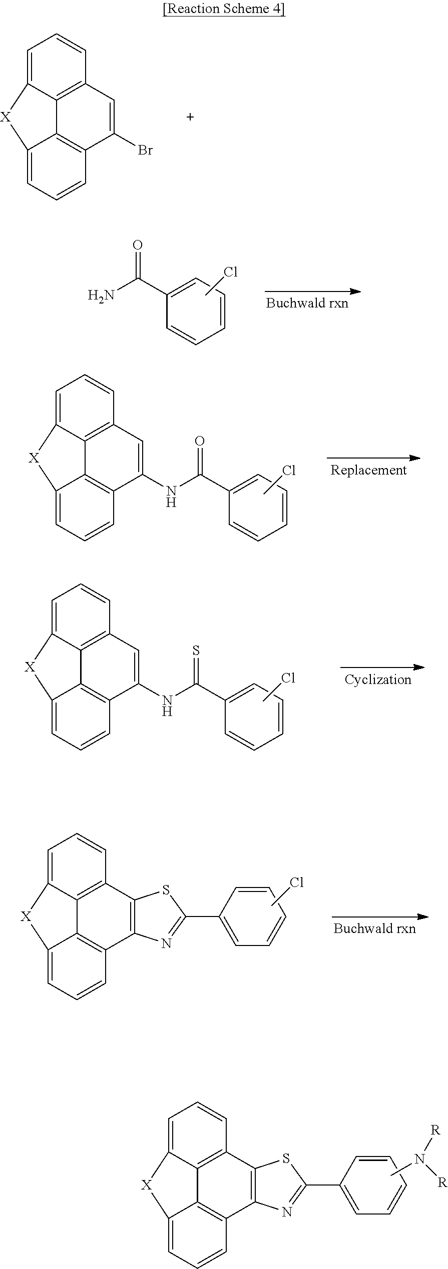

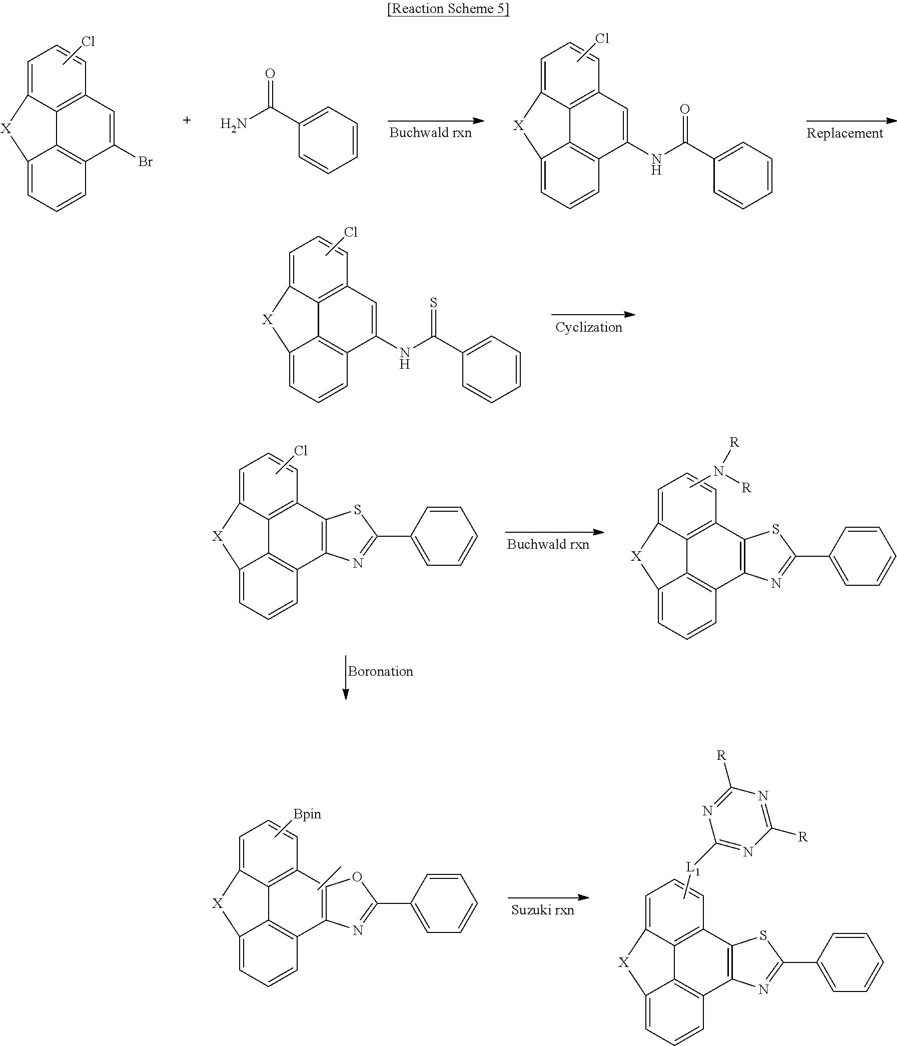

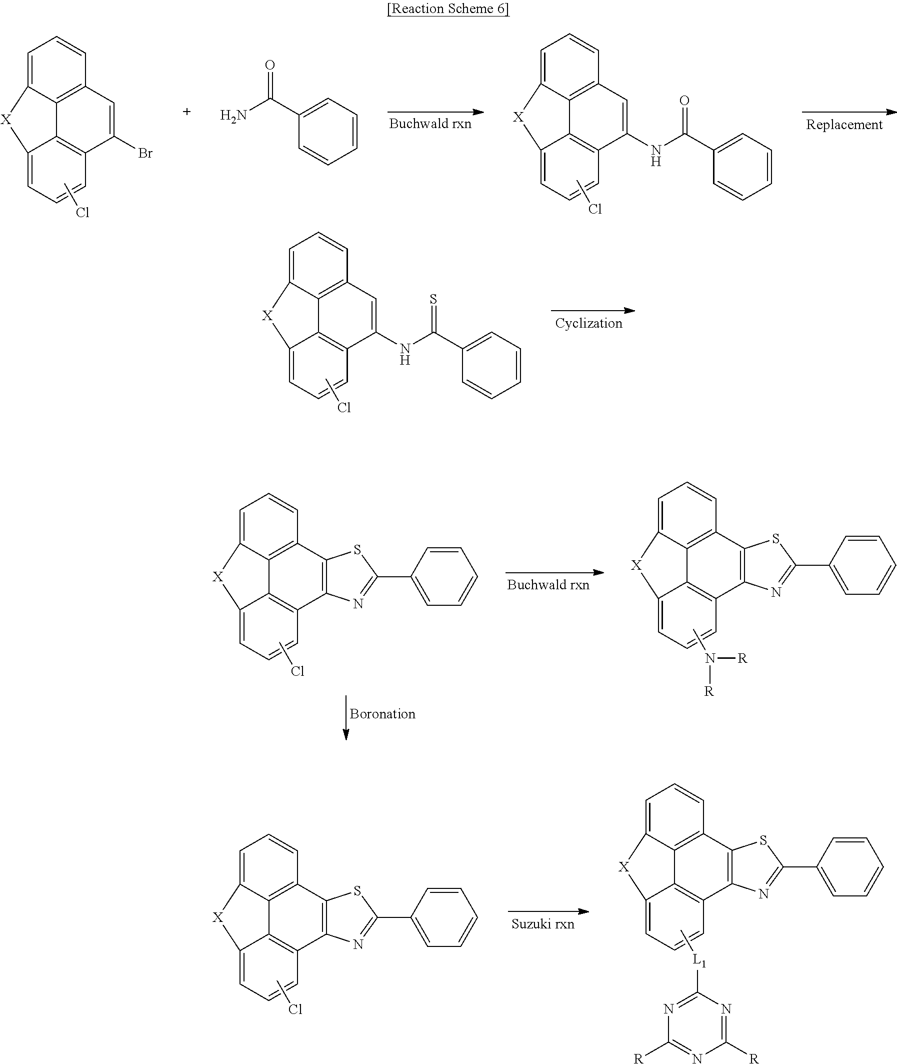

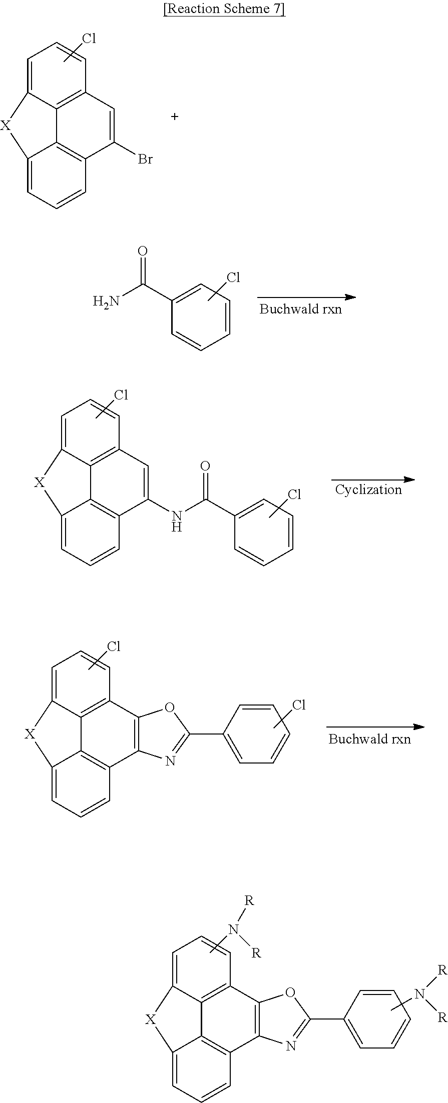

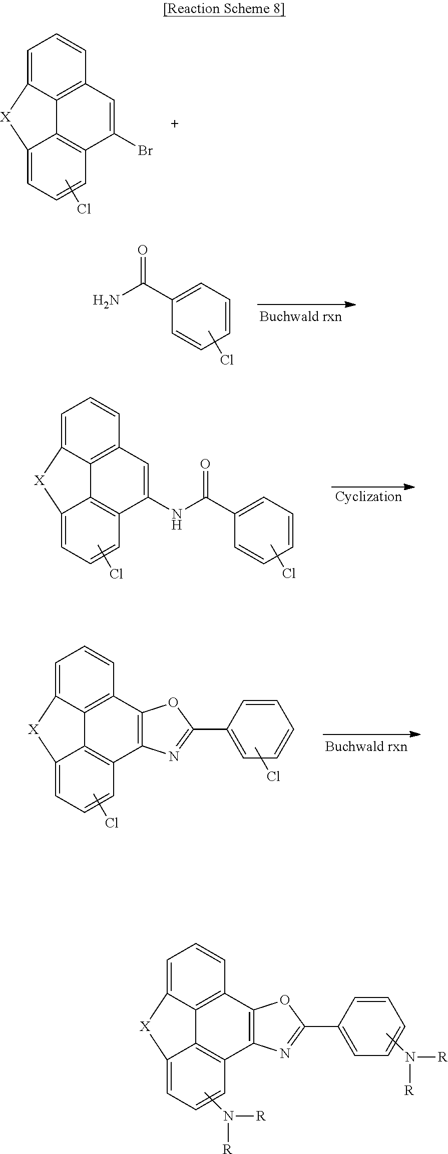

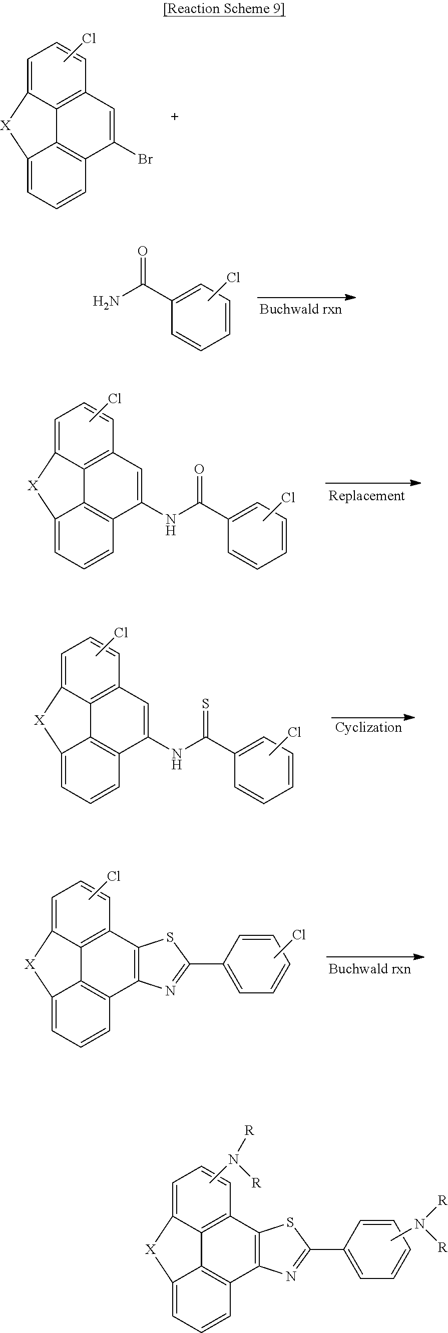

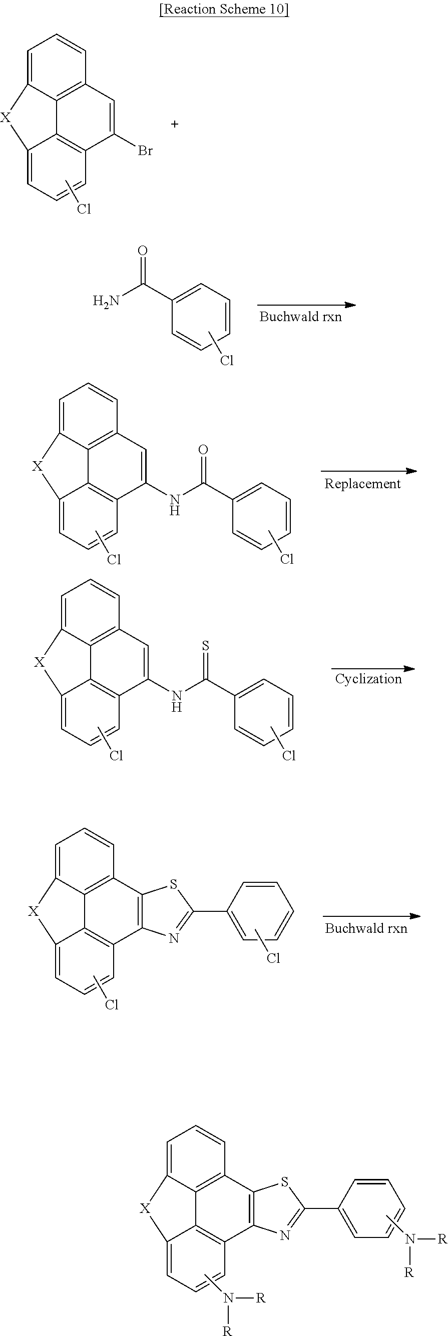

In reaction schemes 1 to 10, X is as defined in formula 1, R is as defined in R′₇ to R′₁₇ of formulas 1-11 to 1-21, or R₂₃ or R₂₄ of formulas 1-31 to 1-33.

The compound represented by formula 2 according to the present disclosure may be produced by a synthetic method known to one skilled in the art, and in particular by using the synthetic methods disclosed in a number of patent literatures, for example, by referring to the methods disclosed in Korean Patent Application Laying-Open No. 2020-0011884, and Korean Patent Application Laying-Open No. 2020-0026083, etc., but is not limited thereto.

Although illustrative synthesis examples of the compounds represented by formulas 1 and 2 of the present disclosure are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN1 substitution reaction, an SN2 substitution reaction, and a Phosphine-mediated reductive cyclization reaction, a Wittig reaction, etc., and the reactions above proceed even when substituents which are defined in formulas 1 and 2 above, but which are not specified in the specific synthesis examples, are bonded.

The present disclosure provides an organic electroluminescent device comprising an anode, a cathode, and at least one light-emitting layer between the anode and cathode in which the at least one light-emitting layer comprises a plurality of host materials according to the present disclosure. The first host material and the second host material according to the present disclosure may be comprised in one light-emitting layer, or may each be comprised in different light-emitting layers. In the plurality of host materials of the present disclosure, the ratio of the compound represented by formula 1 and the compound represented by formula 2 is about 1:99 to about 99:1, preferably about 10:90 to about 90:10, more preferably about 30:70 to about 70:30. In addition, the compound represented by formula 1 and the compound represented by formula 2 in a desired ratio may be combined by mixing them in a shaker, by dissolving them in a glass tube by heat, or by dissolving them in a solvent, etc.

According to one embodiment of the present disclosure, the doping concentration of the dopant compound with respect to the host compound in the light-emitting layer may be less than 20 wt %. The dopant comprised in the organic electroluminescent device of the present disclosure may be at least one phosphorescent or fluorescent dopant, and is preferably a phosphorescent dopant. The phosphorescent dopant material applied to the organic electroluminescent device of the present disclosure is not particularly limited, but may be preferably selected from the group consisting of the metallated complex compounds of iridium (Ir), osmium (Os), copper (Cu), and platinum (Pt), more preferably selected from the group consisting of ortho-metallated complex compounds of iridium (Ir), osmium (Os), copper (Cu), and platinum (Pt), and even more preferably ortho-metallated iridium complex compounds.

The dopant comprised in the organic electroluminescent device of the present disclosure may be a compound represented by the following formula 101, but is not limited thereto.

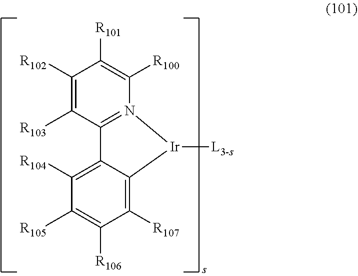

In formula 101,

L is selected from the following structures 1 to 3:

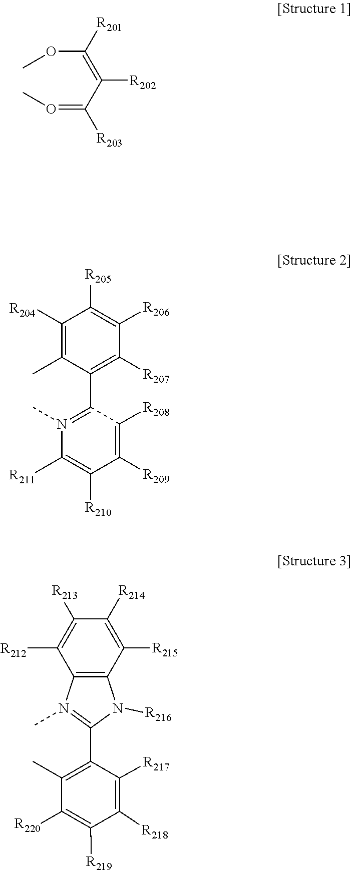

R₁₀₀ to R₁₀₃, each independently represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered) heteroaryl, or a substituted or unsubstituted (C1-C30)alkoxy; or may be linked to an adjacent substituent to form a ring(s), e.g., a substituted or unsubstituted, quinoline, benzofuropyridine, benzothienopyridine, indenopyridine, benzofuroquinoline, benzothienoquinoline, or indenoquinoline, together with pyridine;

R₁₀₄ to R₁₀₇, each independently represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered) heteroaryl, a cyano, or a substituted or unsubstituted (C1-C30)alkoxy; or may be linked to an adjacent substituent to form a substituted or unsubstituted ring(s), e.g., a substituted or unsubstituted, naphthalene, fluorene, dibenzothiophene, dibenzofuran, indenopyridine, benzofuropyridine, or benzothienopyridine, together with benzene;

R₂₀₁ to R₂₂₀, each independently represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent to form a substituted or unsubstituted ring(s); and

s represents an integer of 1 to 3.
The specific examples of the dopant compound are as follows, but are not limited thereto.

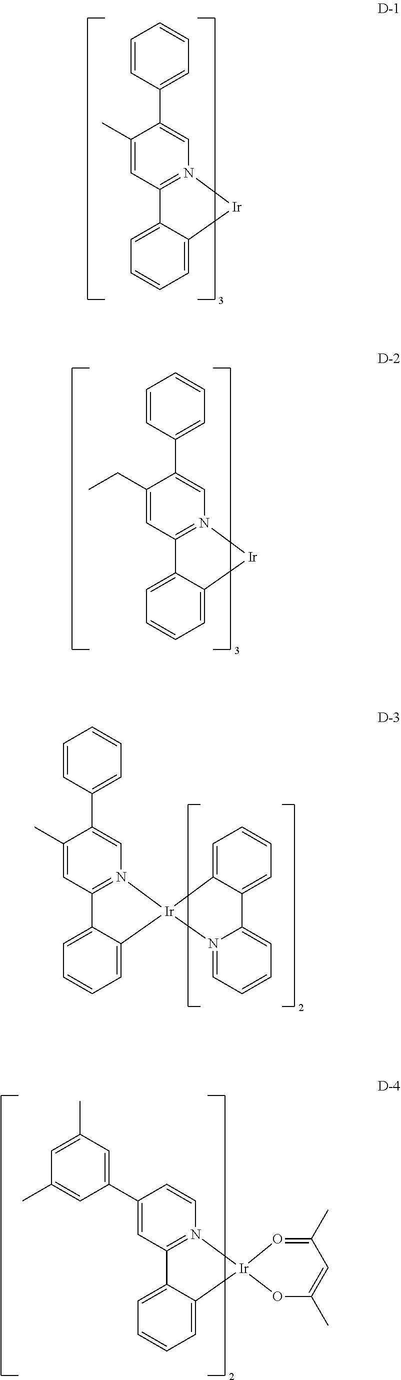

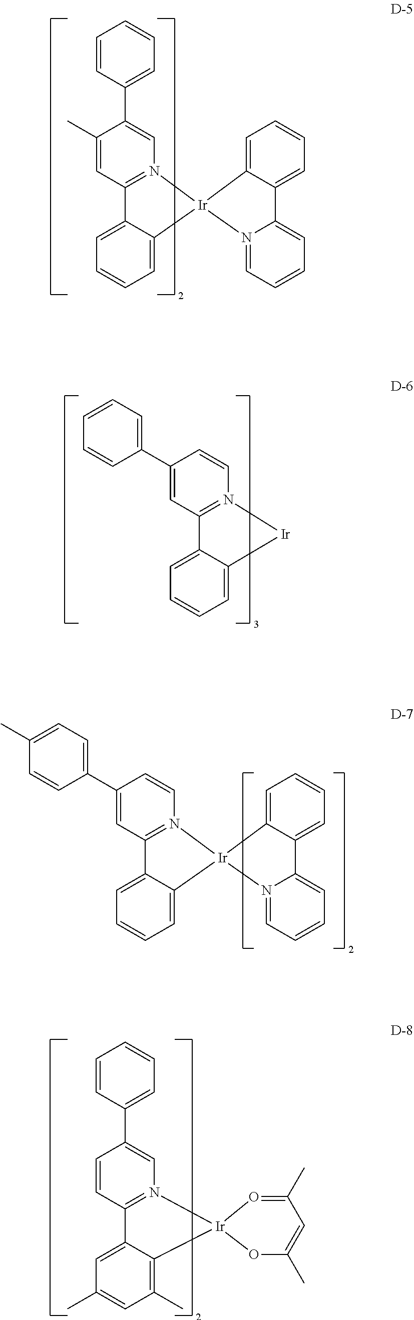

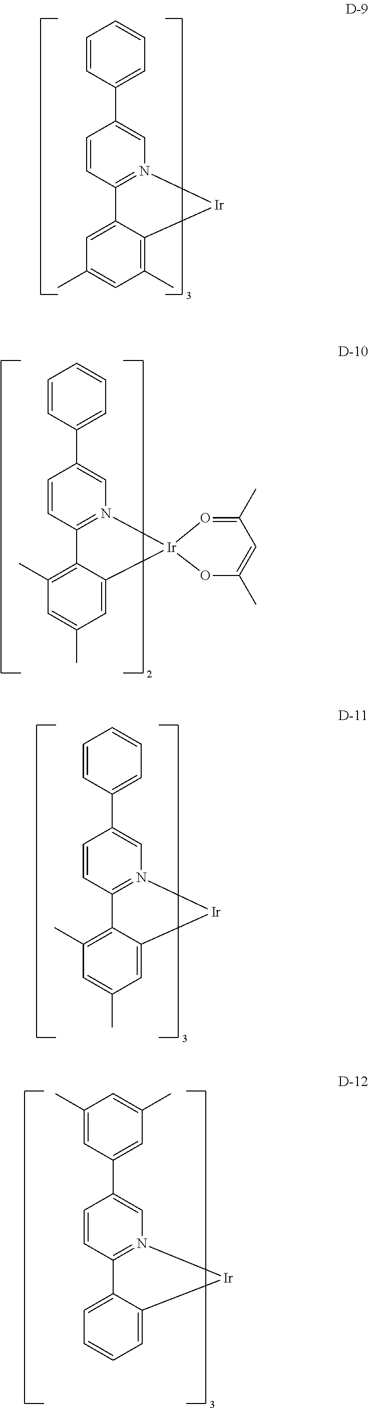

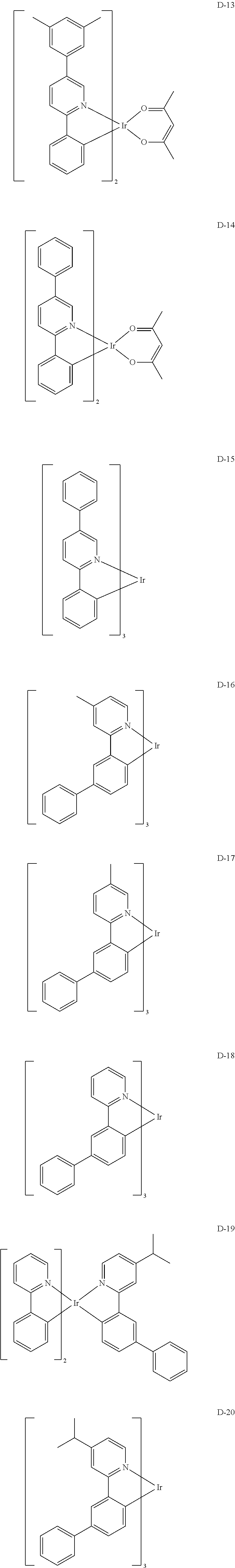

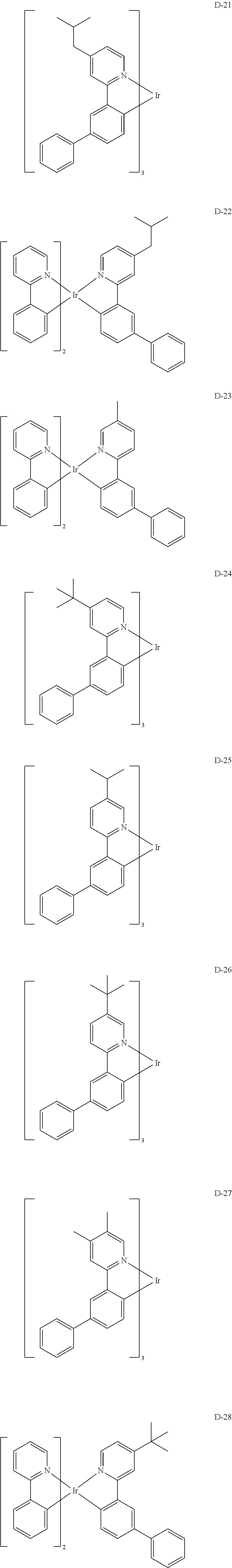

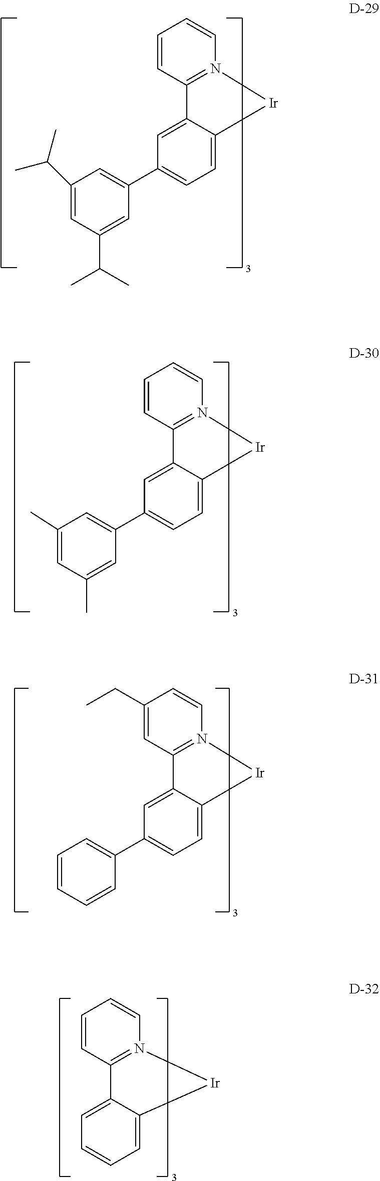

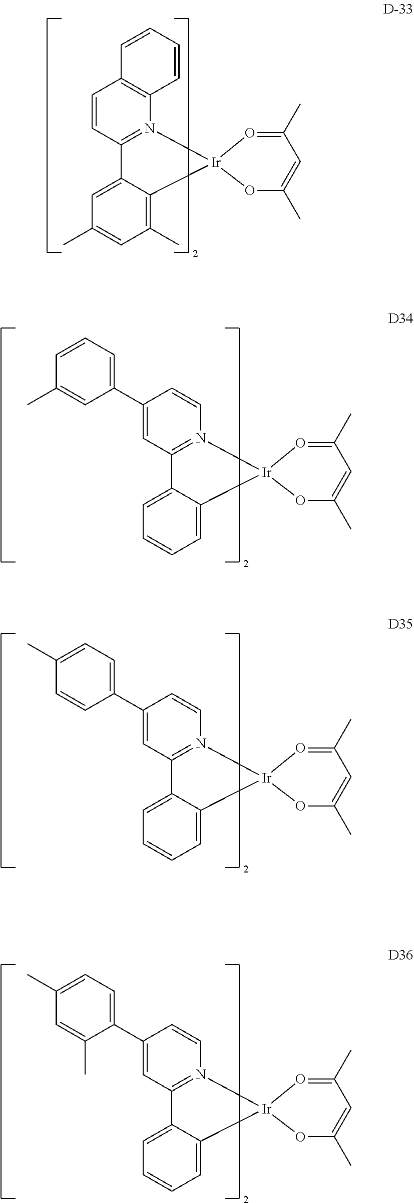

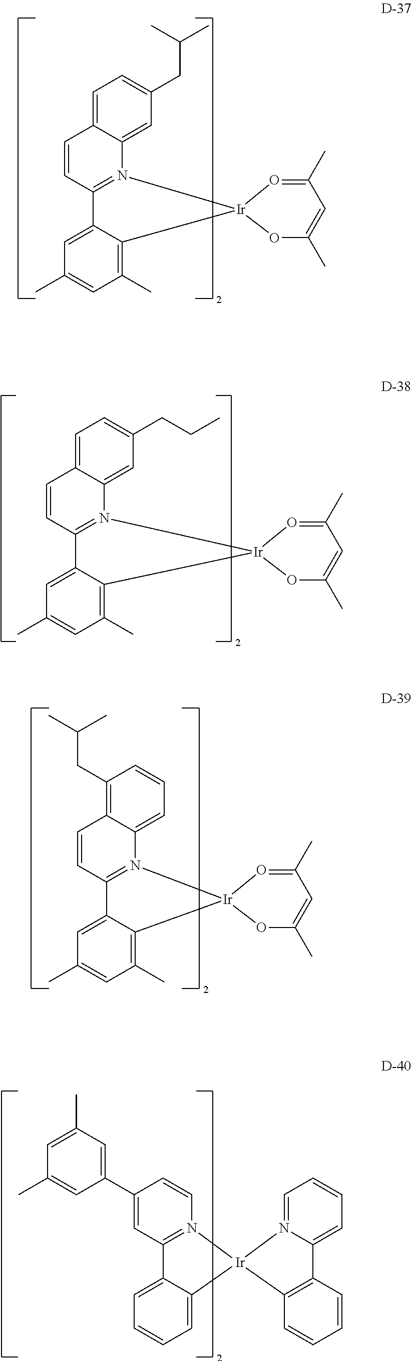

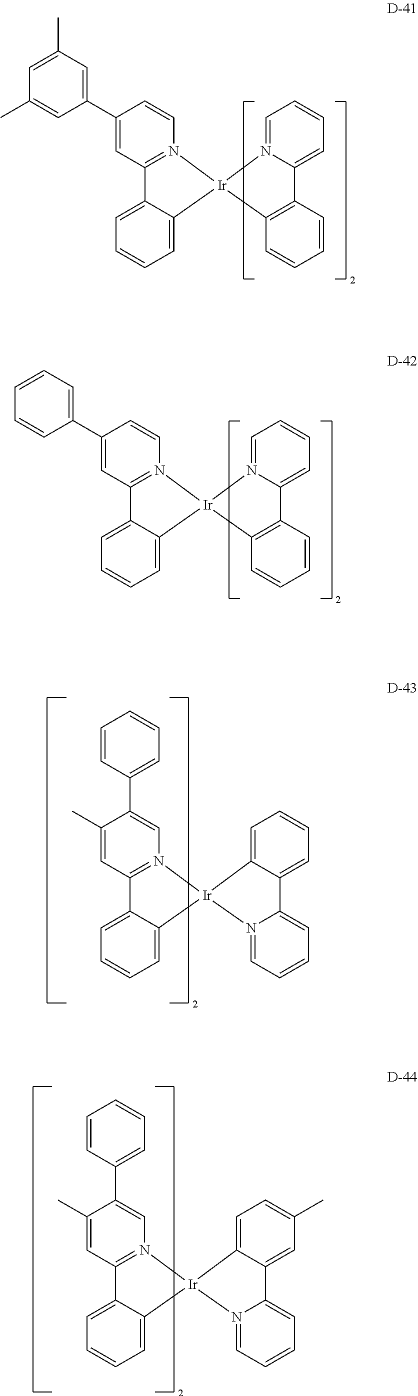

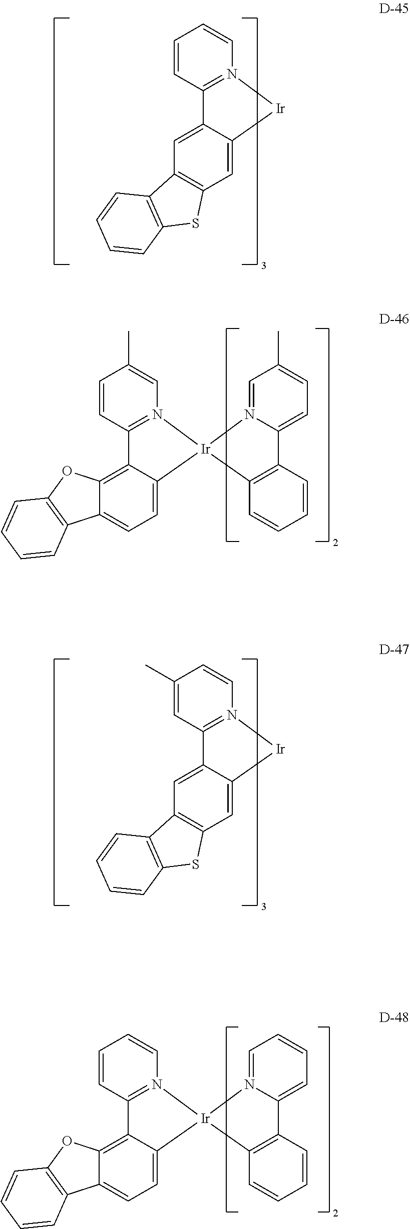

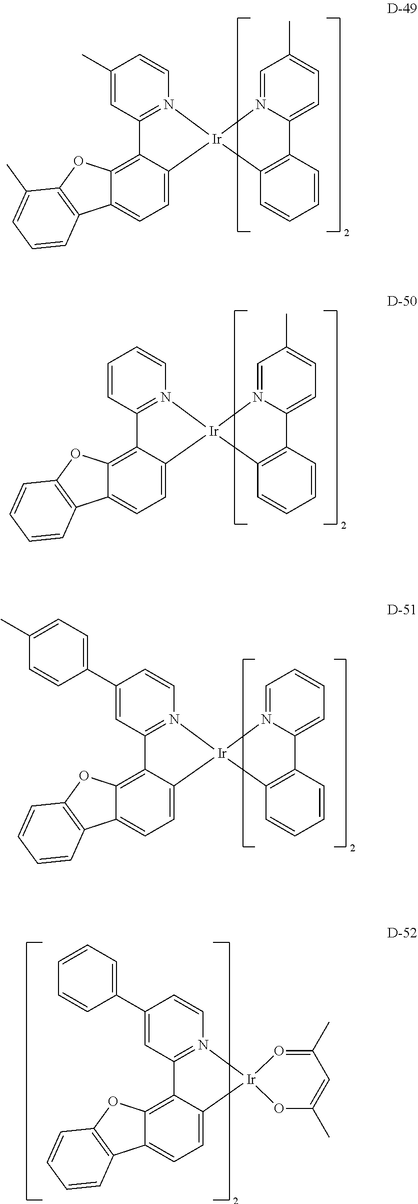

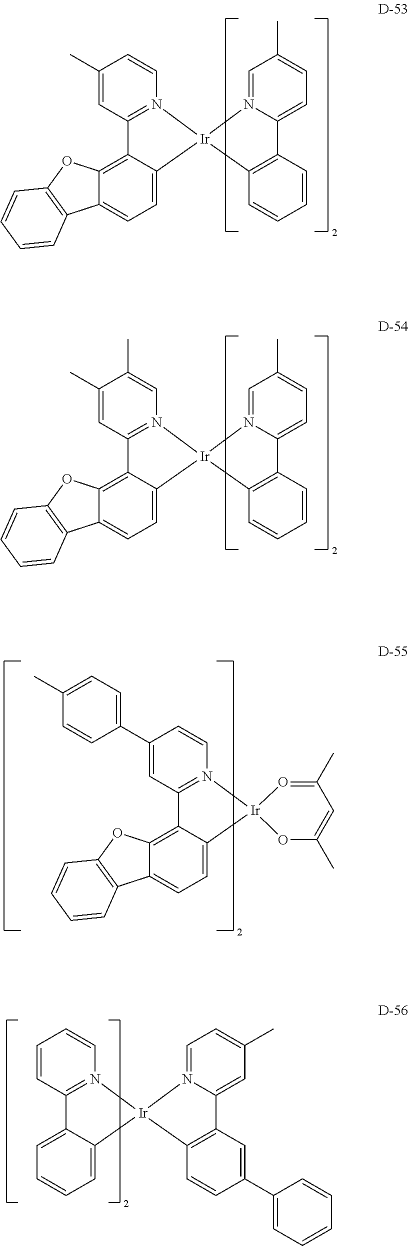

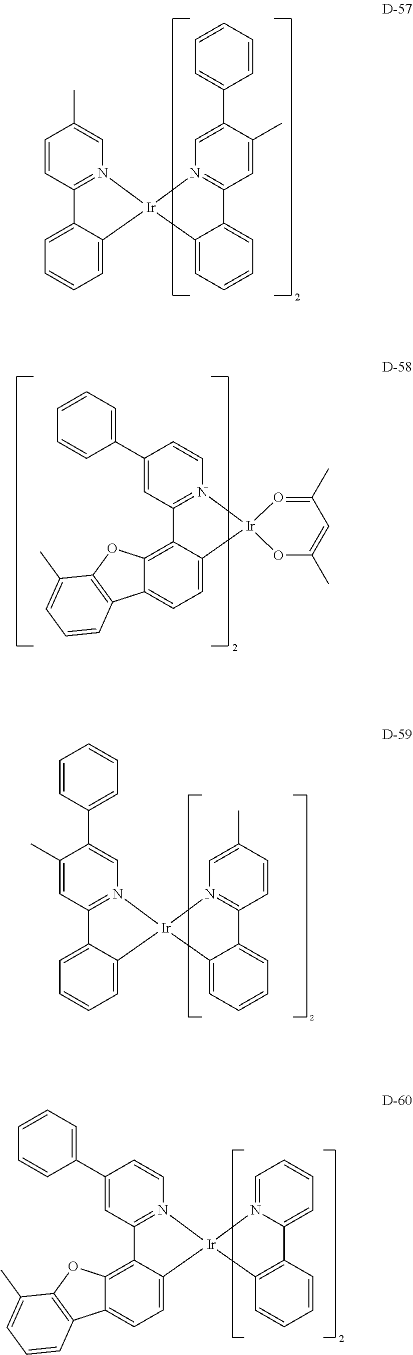

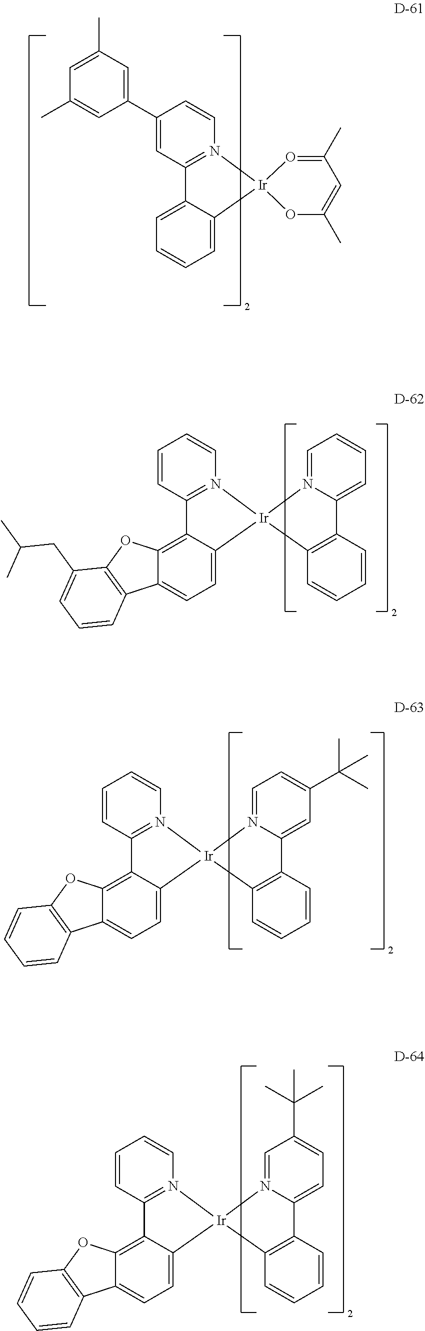

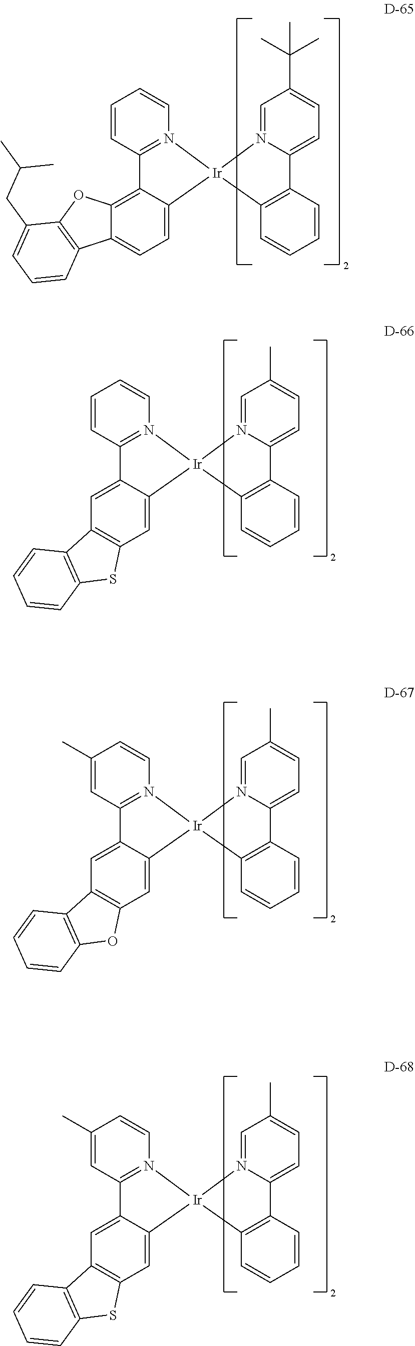

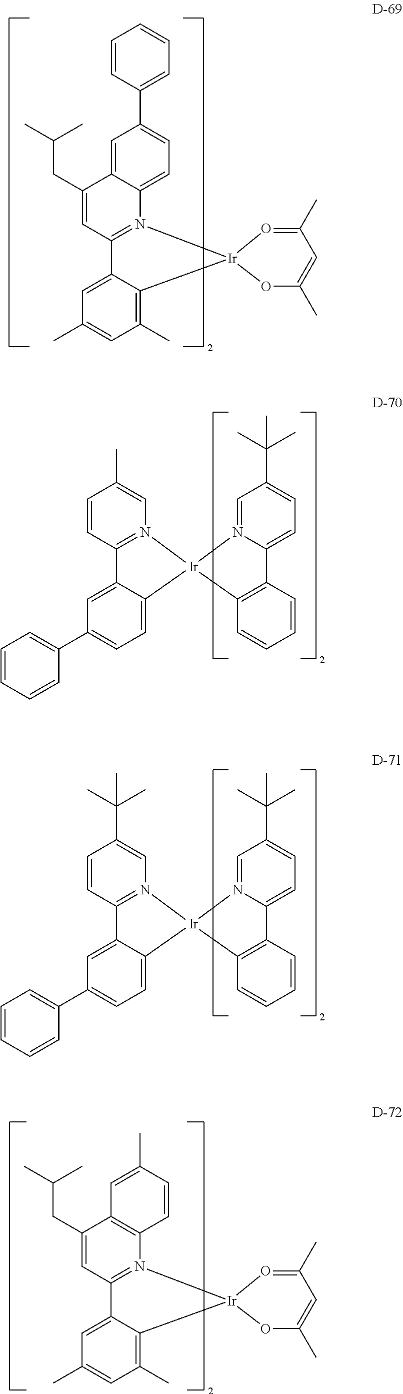

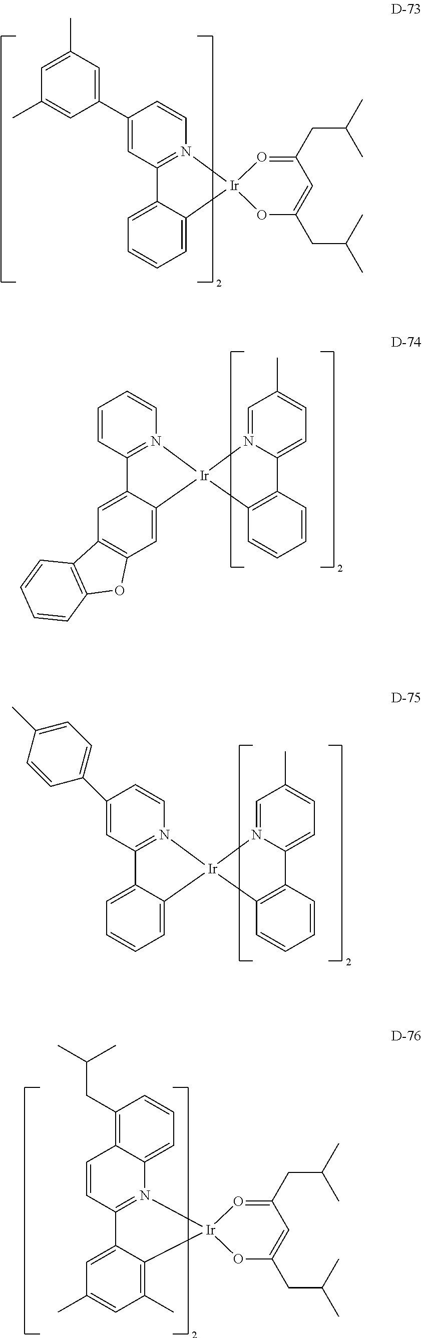

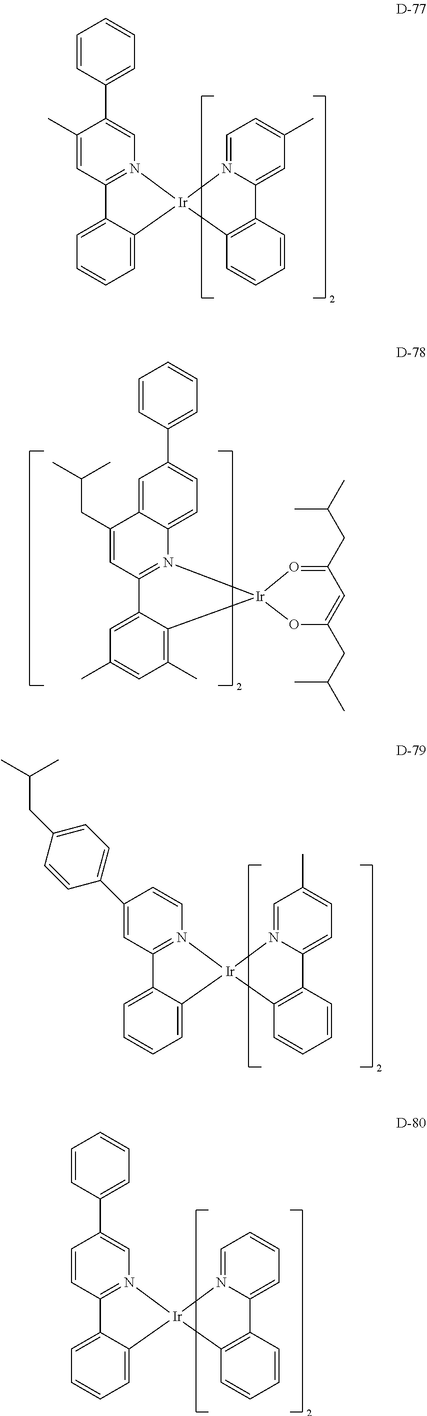

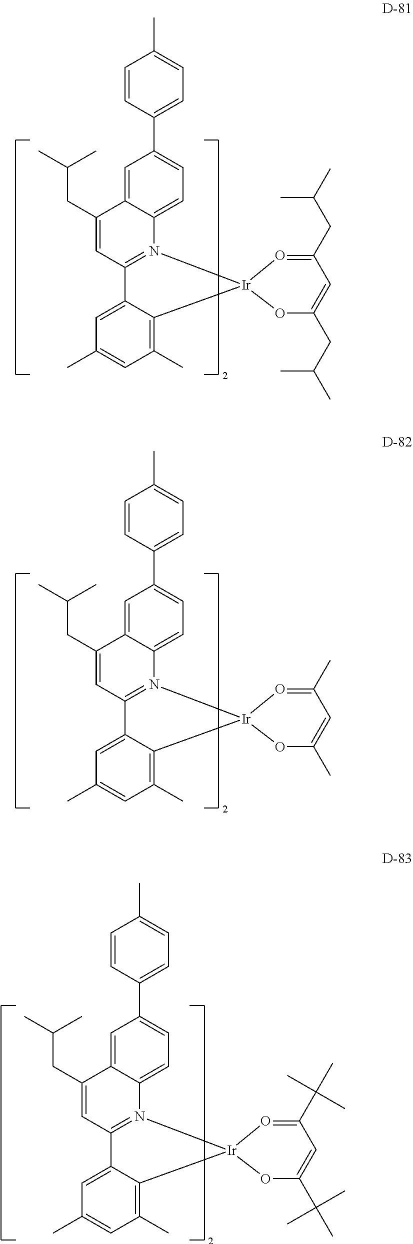

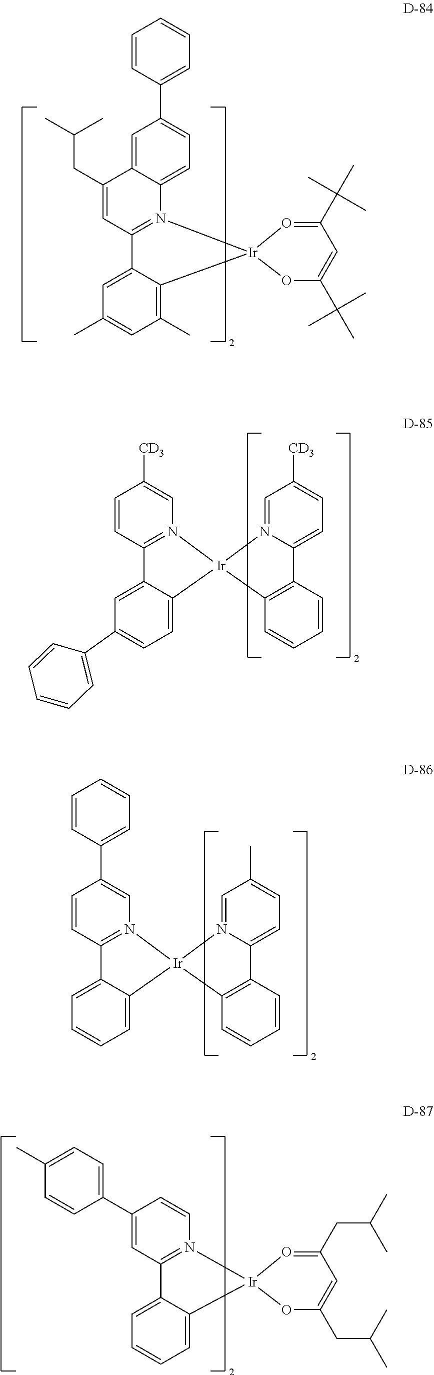

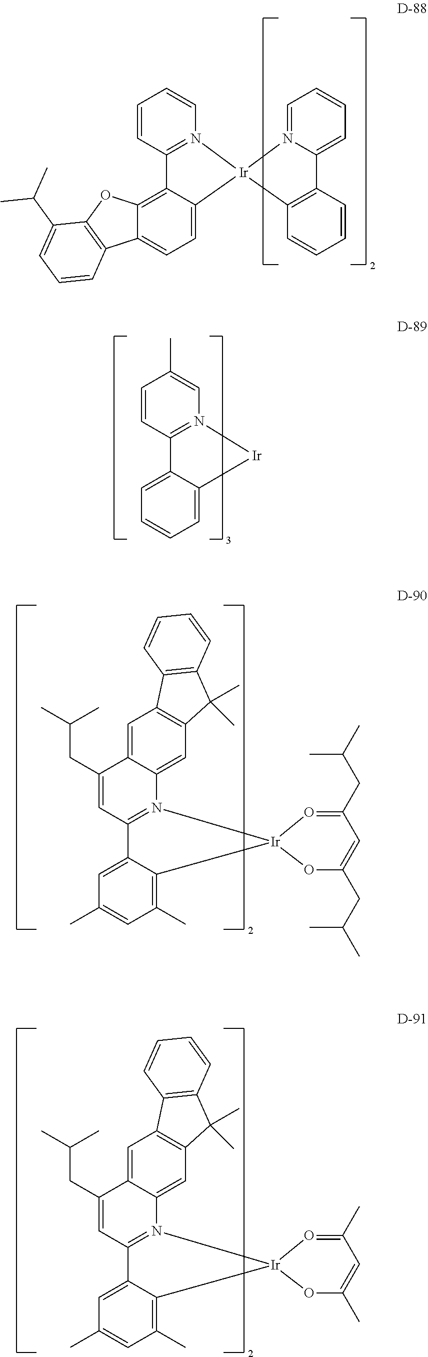

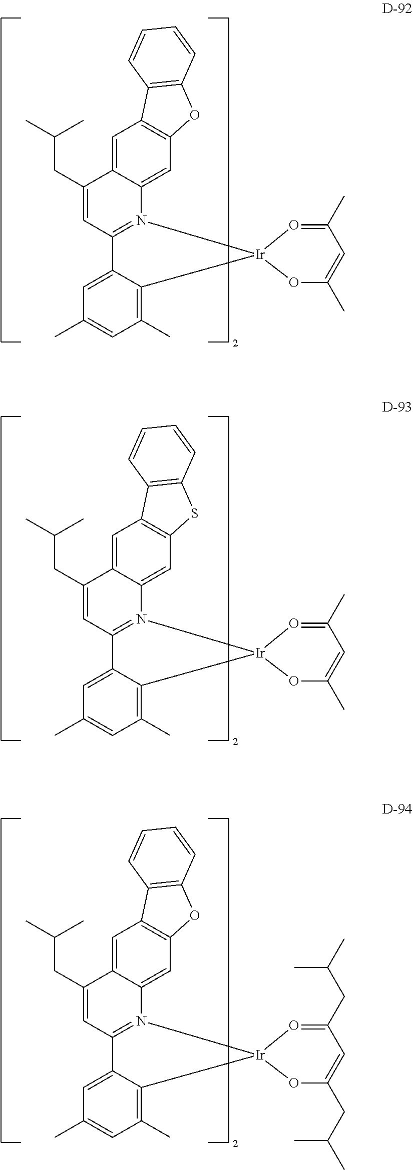

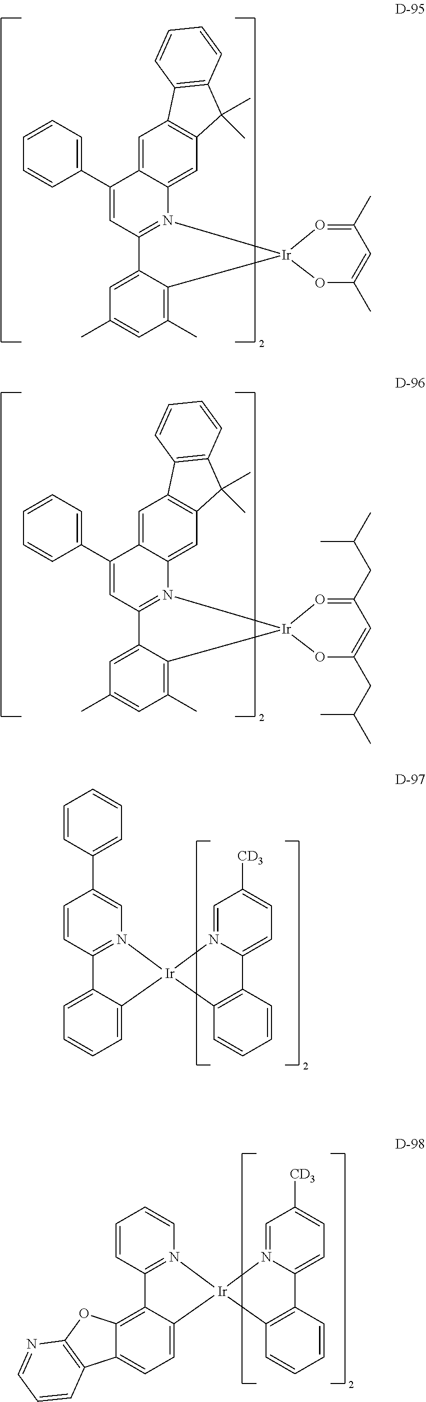

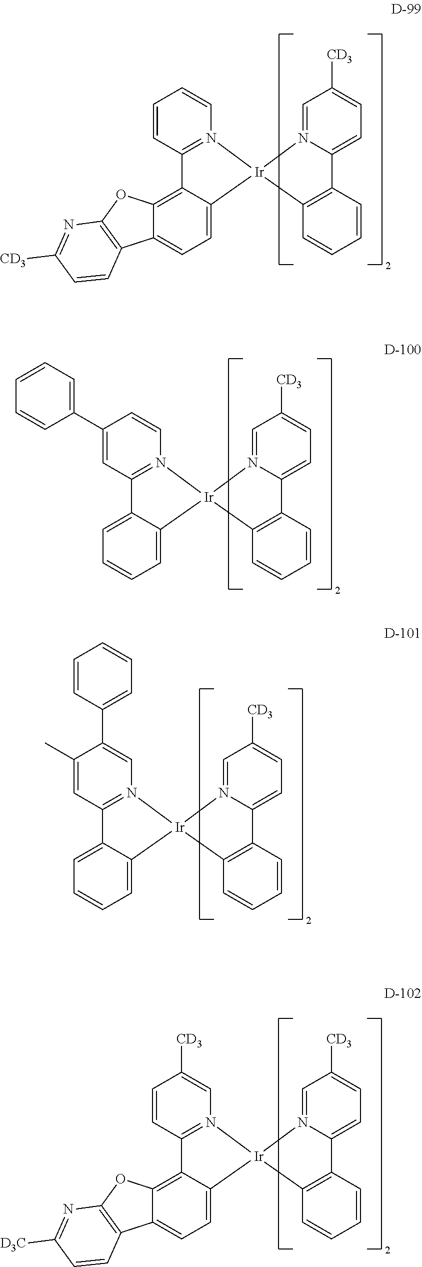

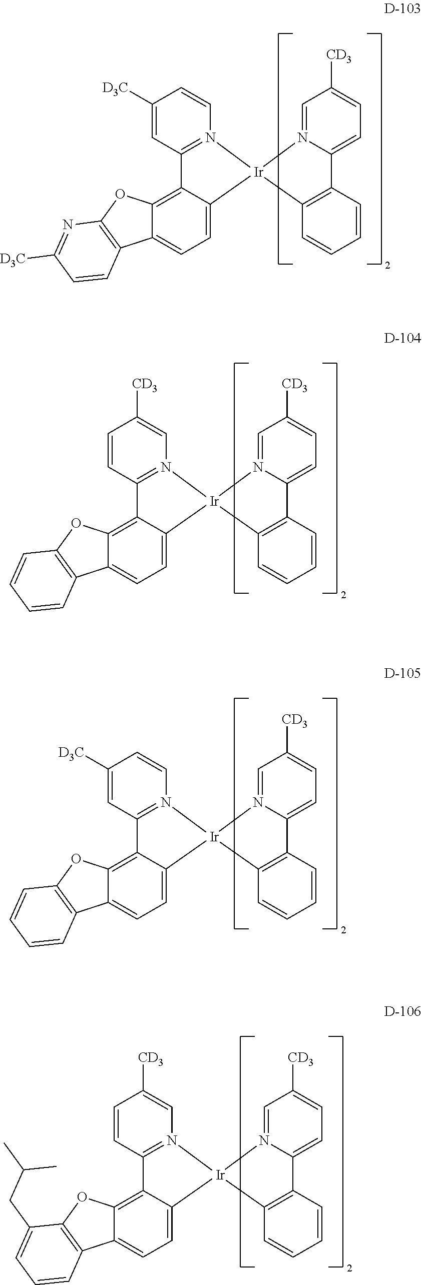

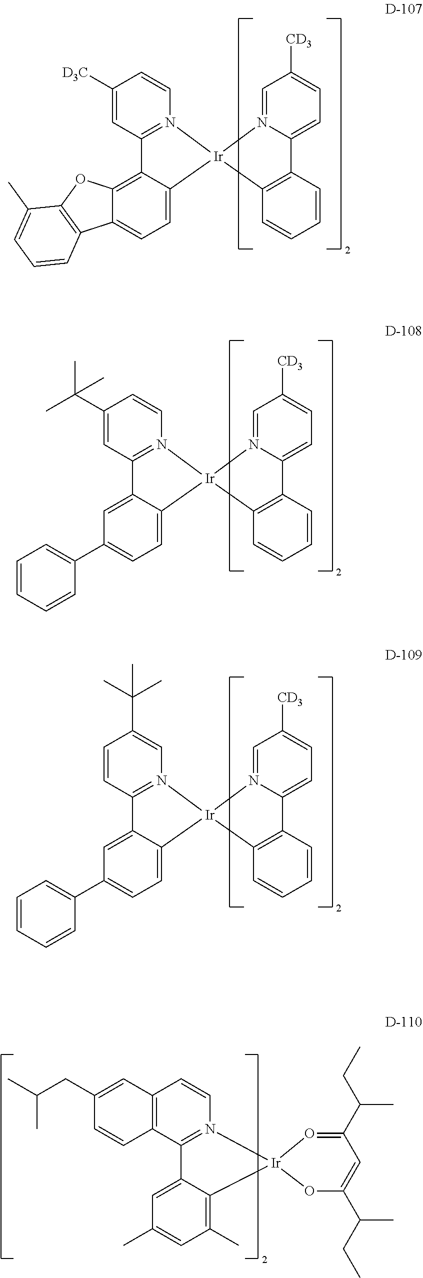

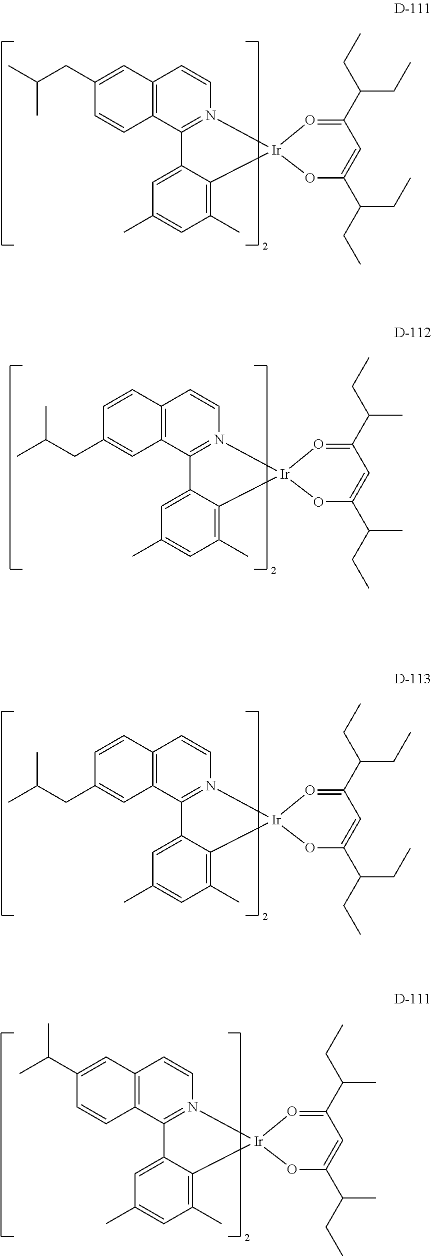

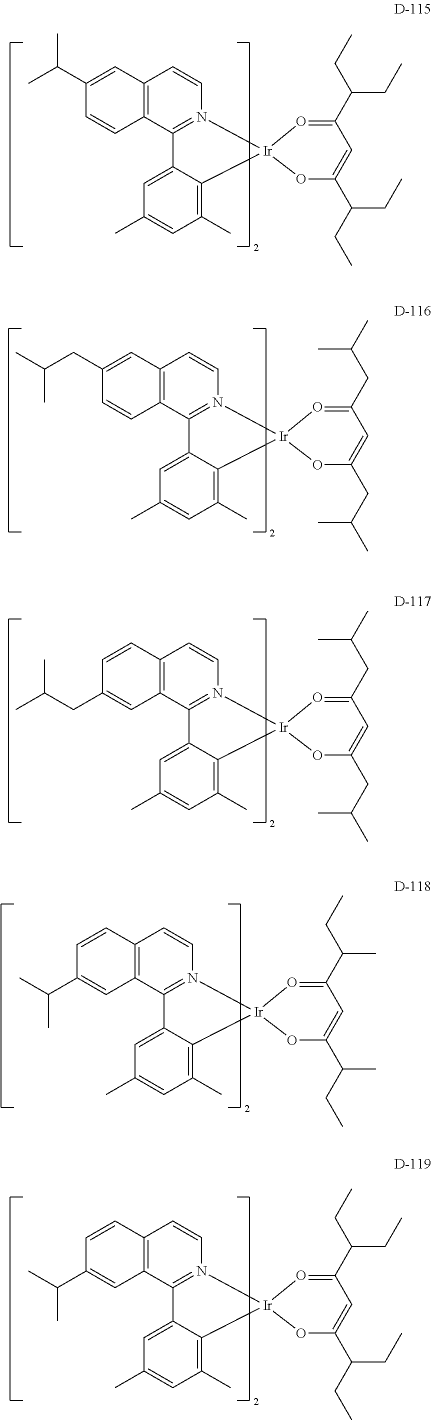

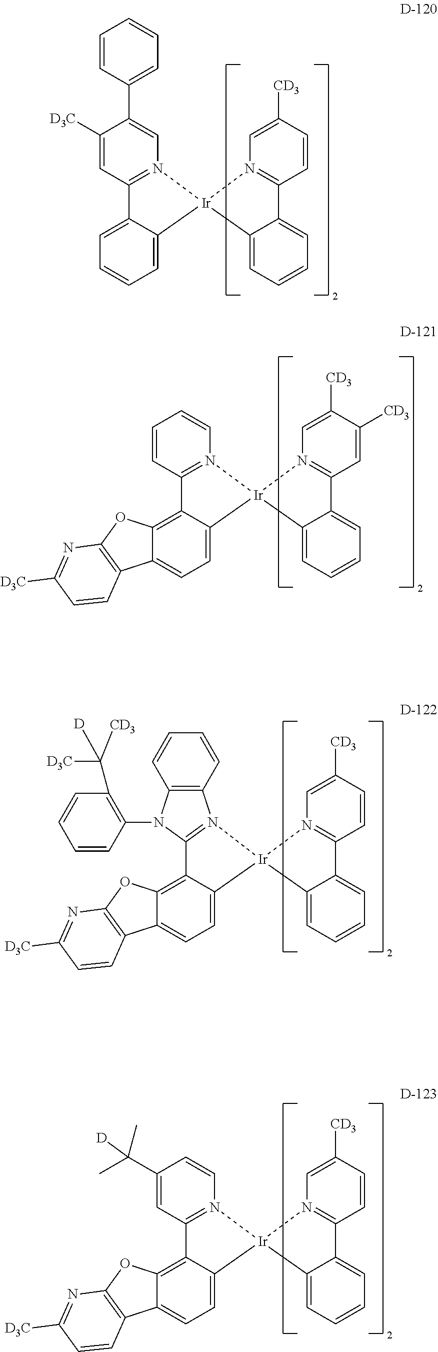

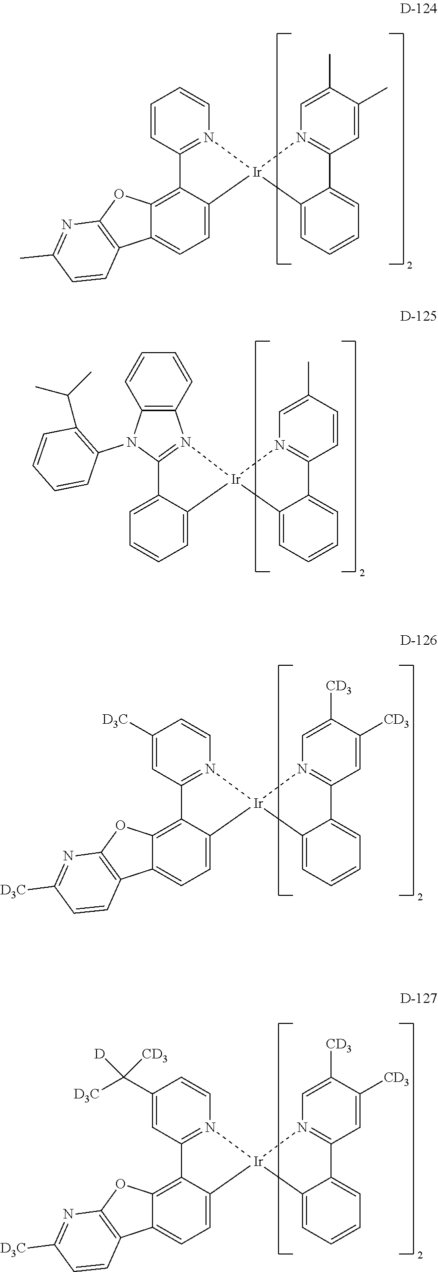

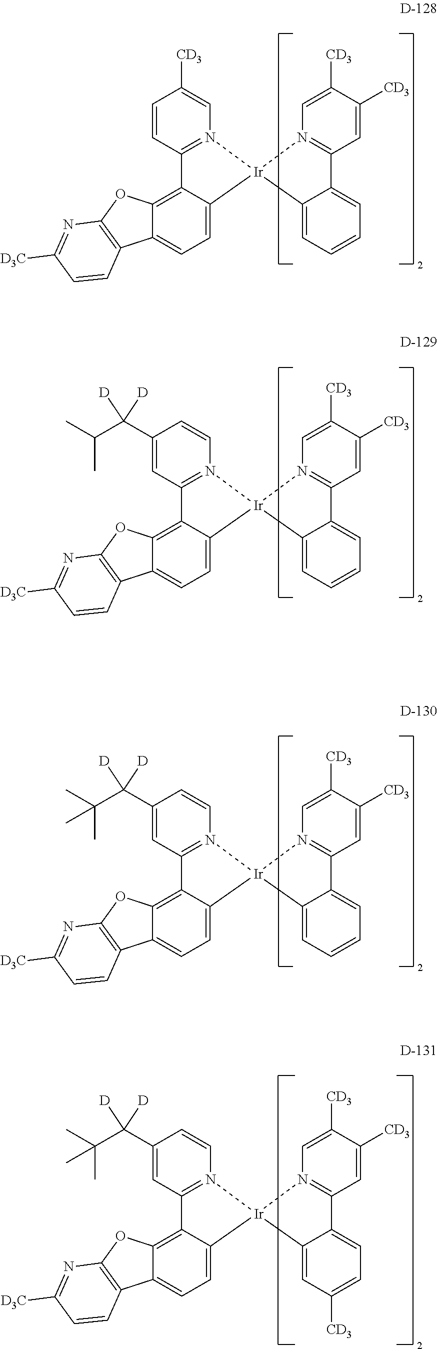

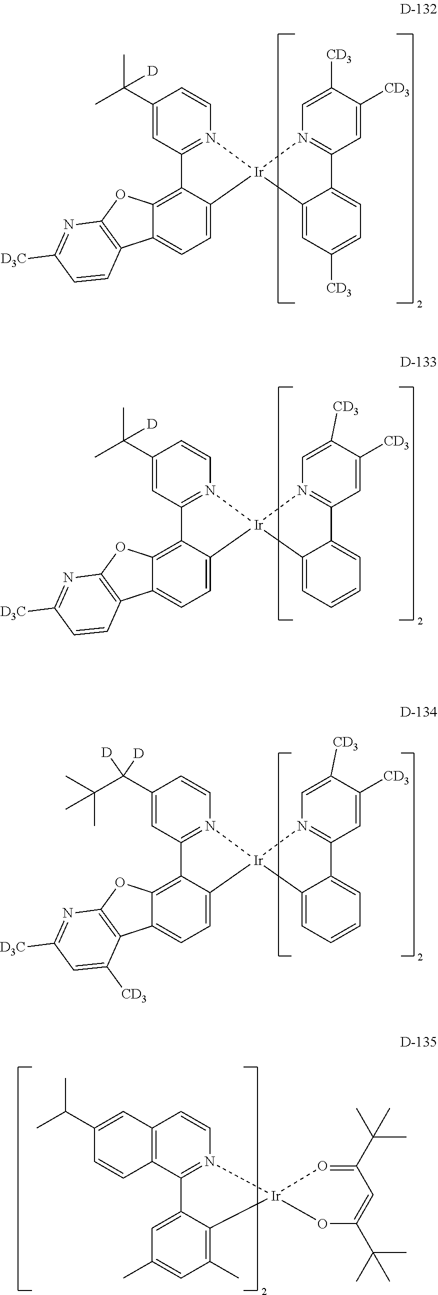

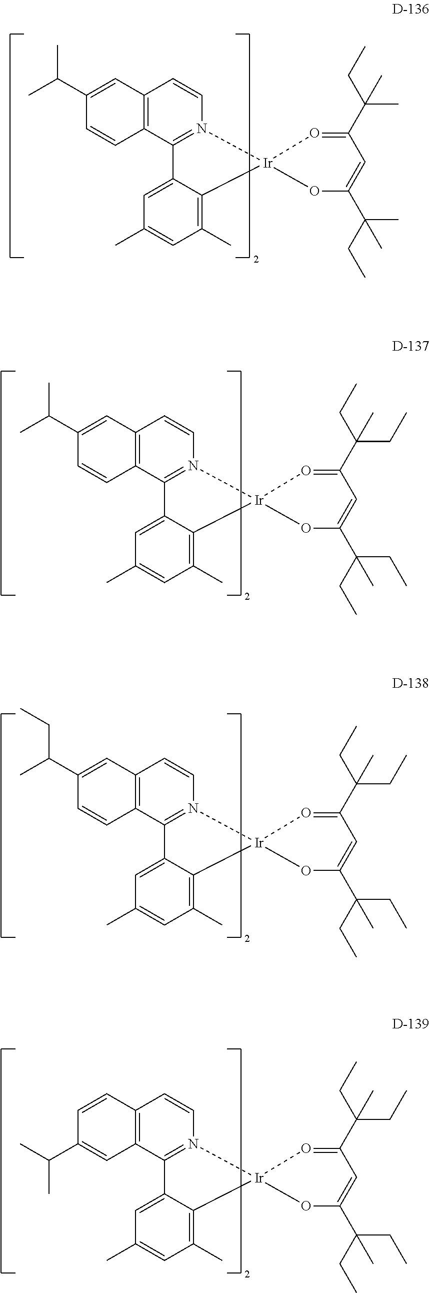

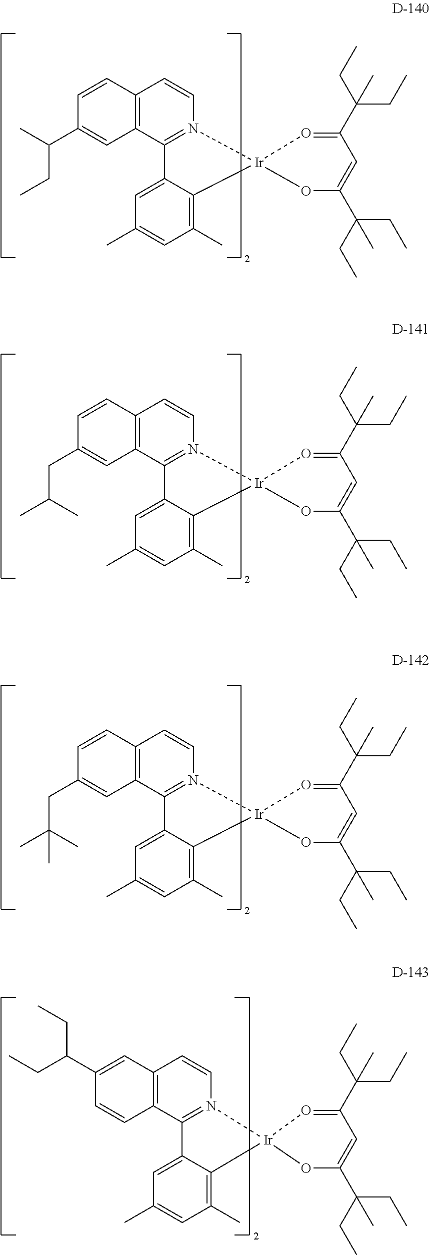

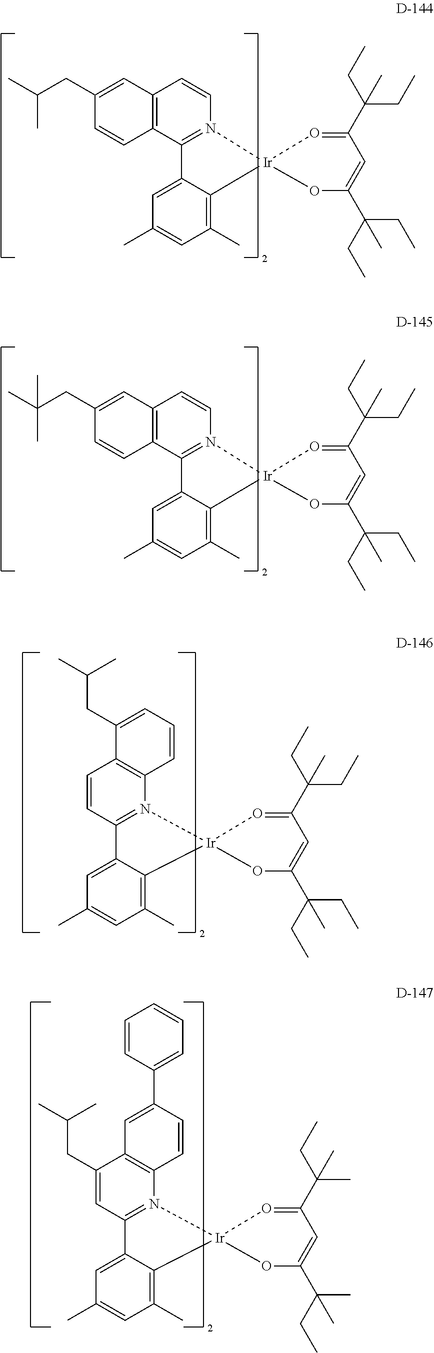

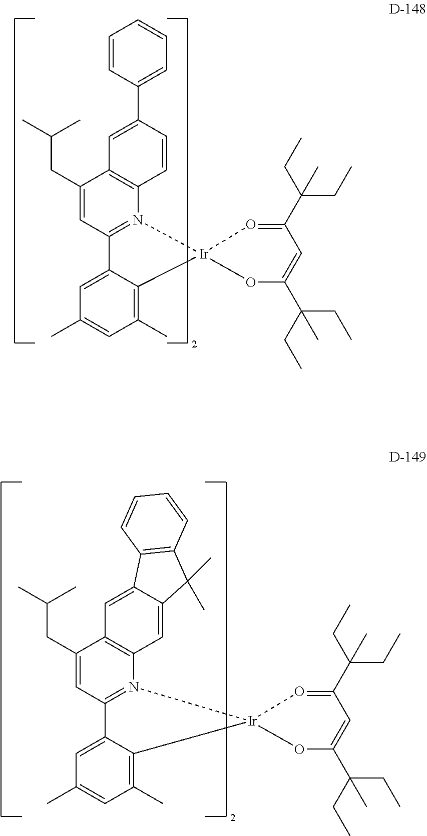

An organic electroluminescent device according to the present disclosure has an anode, a cathode, and at least one organic layer between the anode and the cathode. The organic layer comprises a light-emitting layer and may further comprise at least one layer selected from the group consisting of a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may be further configured as a plurality of layers.

The anode and the cathode may each be formed with a transparent conductive material, or a transflective or reflective conductive material. The organic electroluminescent device may be a top emission type, a bottom emission type, or a both-sides emission type, depending on the materials forming the anode and the cathode. In addition, the hole injection layer may be further doped with a p-dopant, and the electron injection layer may be further doped with an n-dopant.

The organic layer may further comprise at least one compound selected from the group consisting of arylamine-based compounds and styrylarylamine-based compounds. Further, the organic layer may further comprise at least one metal selected from the group consisting of metals of Group 1, metals of Group 2, transition metals of the 4th period, transition metals of the 5th period, lanthanides, and organic metals of the d-transition elements of the Periodic Table, or at least one complex compound comprising the metal.

In addition, the organic electroluminescent device of the present disclosure may emit white light by further comprising at least one light-emitting layer, which comprises a blue, a red, or a green electroluminescent compound known in the field, besides the compound of the present disclosure. If necessary, it may further comprise a yellow or an orange light-emitting layer.

In the organic electroluminescent device of the present disclosure, preferably, at least one layer selected from the group consisting of a chalcogenide layer, a metal halide layer, and a metal oxide layer (hereinafter, “a surface layer”) may be placed on an inner surface(s) of one or both electrode(s). Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, the chalcogenide includes SiOₓ(1≤X≤2), AlOₓ(1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. The hole transport layer or the electron blocking layer may also be multi-layers.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each of the multi-layers may use a plurality of compounds.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or hole transport, or for preventing the overflow of electrons. When the light-emitting auxiliary layer is placed between the cathode and the light-emitting layer, it can be used for promoting the electron injection and/or electron transport, or for preventing the overflow of holes. Also, the hole auxiliary layer may be placed between the hole transport layer (or hole injection layer) and the light-emitting layer, and may be effective to promote or block the hole transport rate (or hole injection rate), thereby enabling the charge balance to be controlled. Further, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifetime of the organic electroluminescent device.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferably, the oxidative dopant includes various Lewis acids and acceptor compounds; and the reductive dopant includes alkali metals, alkali metal compounds, alkaline earth metals, rare-earth metals, and mixtures thereof. The reductive dopant layer may be employed as a charge-generating layer to produce an organic electroluminescent device having two or more light-emitting layers and emitting white light.

An organic electroluminescent device according to the present disclosure may be an organic electroluminescent device having a tandem structure. In the case of the tandem organic electroluminescent device according to one embodiment, a single light-emitting unit (light-emitting part) may be formed in a structure in which two or more units are connected by a charge generation layer. The organic electroluminescent device may include a plurality of two or more light-emitting units, for example, a plurality of three or more light-emitting units, having first and second electrodes opposed to each other on a substrate and a light-emitting layer stacked between the first and second electrodes and emits light in a specific wavelength range. It may include a plurality of light-emitting units, and each of the light-emitting units may include a hole transport zone, a light-emitting layer, and an electron transport zone, and the hole transport zone may include a hole injection layer and a hole transport layer, the electron transport zone may include an electron transport layer and an electron injection layer. According to one embodiment of the present disclosure, three or more light emitting layers may be included in the light emitting unit. A plurality of light-emitting units may emit the same color or different colors. Additionally, one light-emitting unit may include one or more light-emitting layers, the plurality of light-emitting layers may be light-emitting layers of the same or different colors. It may include one or more charge-generation layers located between each light-emitting unit. The charge-generation layer refers to the layer in which holes and electrons are generated when voltage is applied. When there are three or more light-emitting units, a charge-generation layer may be located between each light-emitting unit. At this time, the plurality of charge generation layers may be the same or different from each other. By disposing the charge generating layer between light-emitting units, current efficiency is increased in each light-emitting unit and charges can be smoothly distributed. Specifically, the charge generation layer is provided between two adjacent stacks and can serve to drive a tandem organic electroluminescent device using only a pair of anodes and cathodes without a separate internal electrode located between the stacks.

The charge generation layer may be composed of an N-type charge generation layer and a P-type charge generation layer, and the N-type charge generation layer may be doped with an alkali metal, an alkaline earth metal, or a compound of an alkali metal and an alkaline earth metal, The alkali metal may include one selected from the group consisting of Li, Na, K, Rb, Cs, Fr, Yb, and combinations thereof, and the alkaline earth metal may include one selected from the group consisting of Be, Mg, Ca, Sr, Ba, Ra, and combinations thereof. The P-type charge generation layer may be made of a metal or an organic material doped with a P-type dopant. For example, the metal may be made of one or two or more alloys selected from the group consisting of Al, Cu, Fe, Pb, Zn, Au, Pt, W, In, Mo, Ni, and Ti. Additionally, commonly used materials may be used as the P-type dopant and host materials used in the P-type doped organic material.

The organic electroluminescent material according to one embodiment of the present disclosure may be used as a light-emitting material for a white organic light-emitting device. The white organic light-emitting device has been suggested to have various structures such as a side-by-side arrangement method, a stacking arrangement method, or a color conversion material (CCM) method, etc., according to the arrangement of R (Red), G (Green) or YG (Yellowish Green), and B (Blue) light-emitting units. In addition, the organic electroluminescent material according to one embodiment of the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used. When forming a film of the first host compound and the second host compound of the present disclosure, co-deposition or mixed deposition is performed.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

The manufacturing method of the organic electroluminescent device of the present disclosure is not limited, and the manufacturing method of the Device Example as described below is only an example and is not limited thereto. One skilled in the art can reasonably modify the manufacturing method of the Device Examples as described below by relying on existing technology. For example, there is no particular limitation on the mixing ratio of the first compound and the second compound, and thus one skilled in the art can reasonably select it within a certain range by depending on existing technology. For example, based on the total weight of the light-emitting layer material, the total weight of the first compound and the second compound accounts for 99.5%-80.0% of the total weight of the light-emitting layer, the weight ratio of the first compound and the second compound is between 1:99 and 99:1, the weight ratio of the first compound and the second compound may be between 20:80 and 99:1, or the weight ratio of the first compound and the second compound may be between 50:50 and 90:10. In the manufacture of devices, when forming a light-emitting layer by co-depositing two or more host materials and a light-emitting material, the two or more host materials and the light-emitting material may each be placed in different evaporation sources and co-deposited to form a light-emitting layer, or a pre-mixed mixture of two or more host materials may be placed on the same evaporation source and then co-deposited with a light-emitting material placed on another evaporation source to form a light-emitting layer. This premixing method can further save evaporation sources. According to one embodiment, the first compound, the second compound, and the light-emitting material of the present disclosure may each be placed in different evaporation sources and co-deposited to form a light-emitting layer, or a pre-mixed mixture of the first compound and the second compound may be placed in the same evaporation source and then co-deposited with a light-emitting material placed in another evaporation source to form a light-emitting layer.

In addition, it is possible to produce a display system, for example, a display system for smart phones, tablets, notebooks, PCs, TVs, or cars; or a lighting system, for example an outdoor or indoor lighting system, by using the organic electroluminescent device of the present disclosure.

Hereinafter, the preparation method of the compounds according to the present disclosure and the properties thereof, and the driving voltage and the luminous efficiency of an organic electroluminescent device (OLED) comprising a plurality of host materials according to the present disclosure will be explained in detail with reference to the representative compounds of the present disclosure. However, the following examples only describe the properties of the compound according to the present disclosure and the OLED comprising the same, and the present disclosure is not limited to the following examples.

### Example 1: Preparation of Compound C-10

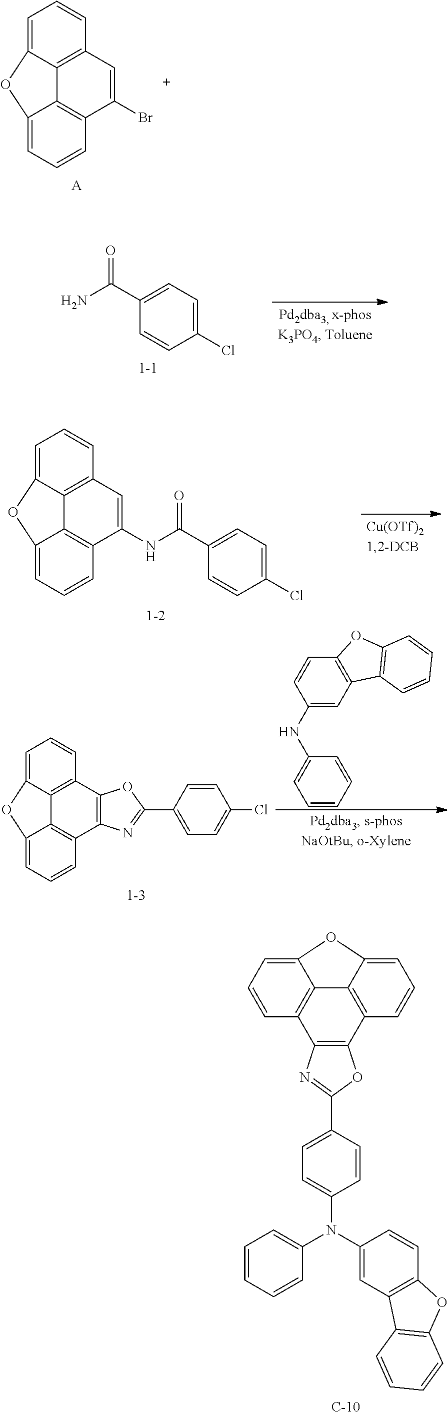

### 1) Synthesis of Compound 1-2

Compound A (12 g, 44.28 mmol), Compound 1-1 (7.58 g, 48.71 mmol), Pd₂dba₃ (2.84 g, 3.10 mmol), x-phos (2.96 g, 6.20 mmol), K₃PO₄ (18.8 g, 88.56 mmol), and 220 mL of toluene were added to a flask and stirred at 140° C. for 2 hours. After completion of the reaction, the reactant was cooled to room temperature, and separated by column chromatography to obtain Compound 1-2 (15 g, yield: 98%).

### 2) Synthesis of Compound 1-3

Compound 1-2 (9 g, 26.03 mmol), Cu(OTf)₂ (14.1 g, 39.04 mmol), and 330 ml of 1,2-DCB were added to a flask, and stirred at 160° C. for 12 hours. After completion of the reaction, the mixture was distilled to concentrate the organic matter and separated by column chromatography to obtain Compound 1-3 (1.5 g, yield: 16%).

### 3) Synthesis of Compound C-10

Compound 1-3 (2 g, 3.5 mmol), N-phenyldibenzo[b,d]furan-2-amine (1.1 g, 4.22 mmol), Pd₂dba₃ (193 mg, 0.21 mmol), s-phos (173 mg, 0.42 mmol), NaOtBu (1.0 g, 10.55 mmol), and 30 mL of o-xylene were added to a flask, and stirred at 150° C. for 4 hours. After completion of the reaction, the reactant was cooled to room temperature and separated by column chromatography to obtain Compound C-10 (1.3 g, yield: 54%).

| | MW | M.P |
|---|---|---|
| C-10 | 566.62 | 231° C. |

### Example 2: Preparation of Compound C-15

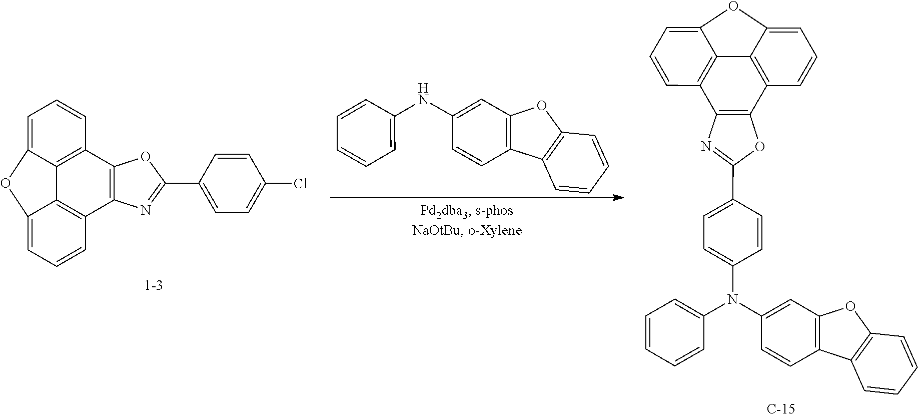

Compound 1-3 (1.74 g, 5.06 mmol), N-phenyldibenzo[b,d]furan-2-amine (1.31 g, 5.06 mmol), Pd₂dba₃ (230 mg, 0.25 mmol), s-phos (207 mg, 0.51 mmol), NaOtBu (1.21 g, 12.65 mmol), and 35 mL of o-xylene were added to a flask and stirred at 150° C. for 2 hours. After completion of the reaction, the mixture was cooled to room temperature, and separated by column chromatography to obtain Compound C-15 (1.8 g, yield: 63%).

| | MW | M.P. |
|---|---|---|
| C-15 | 566.62 | 304° C. |

### Example 3: Preparation of Compound C-13

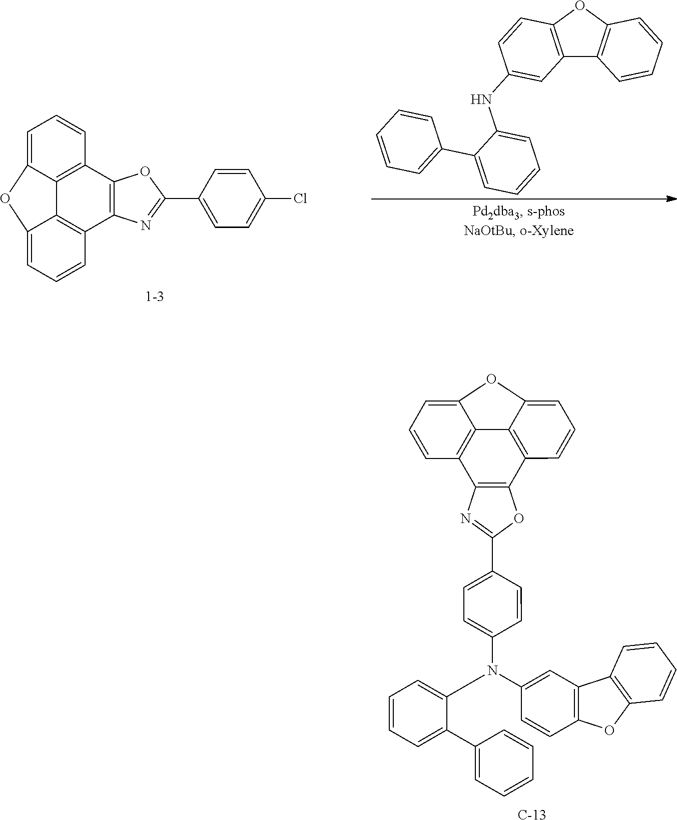

Compound 1-3 (1.3 g, 3.78 mmol), N-([1,1′-biphenyl]-2-yl)dibenzo[b,d]furan-2-amine (1.33 g, 3.97 mmol), Pd₂dba₃ (173 mg, 0.19 mmol), s-phos (155 mg, 0.38 mmol), NaOtBu (908 mg, 9.45 mmol), and 25 mL of o-xylene were added to a flask, and stirred at 160° C. for 2 hours. After completion of the reaction, the reactant was cooled to room temperature, and separated by column chromatography to obtain Compound C-13 (1.4 g, yield: 58%).

| | MW | M.P. |
|---|---|---|
| C-13 | 642.71 | 275° C. |

### Example 4: Preparation of Compound C-3

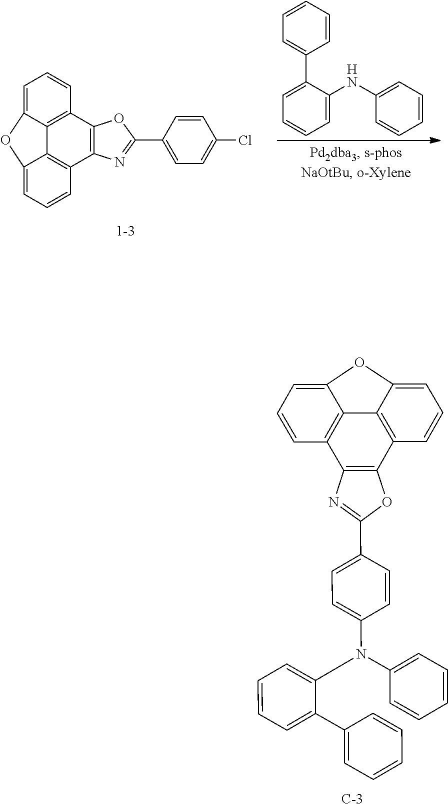

Compound 1-3 (1.55 g, 4.51 mmol), N-phenyl-[1,1′-biphenyl]-2-amine (1.55 g, 4.51 mmol), Pd₂dba₃ (206 mg, 0.23 mmol), s-phos (185 mg, 0.45 mmol), NaOtBu (1.08 g, 11.28 mmol), and 30 mL of o-xylene were added to a flask and stirred at 160° C. for 3 hours. After completion of the reaction, the mixture was cooled to room temperature and separated by column chromatography to obtain Compound C-3 (1.37 g, yield: 55%).

| | MW | M.P. |
|---|---|---|
| C-3 | 552.63 | 178° C. |

### Example 5: Preparation of Compound C-63

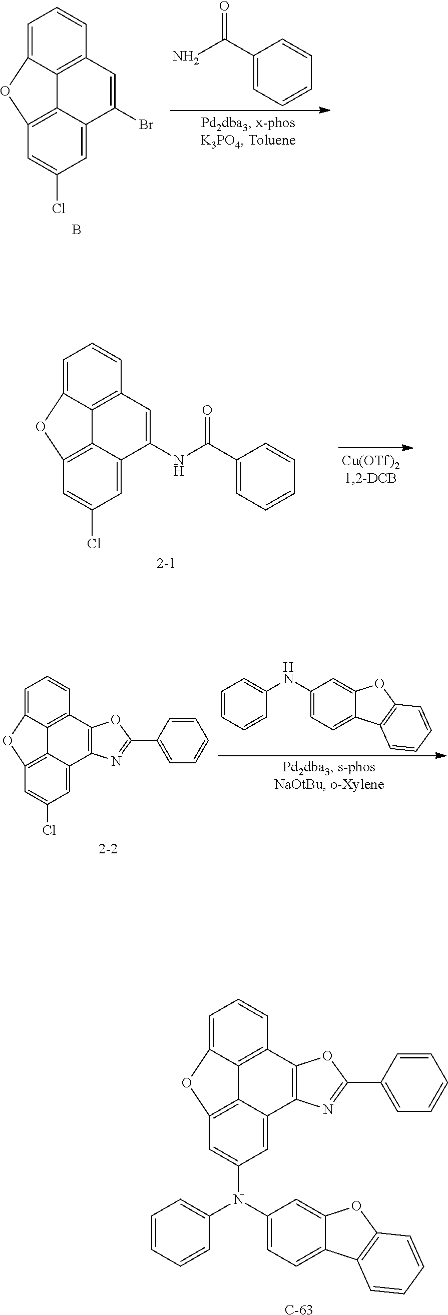

### 1) Synthesis of Compound 2-1

Compound B (50 g, 163.6 mmol), benzamide (21.8 g, 180.0 mmol), Pd₂dba₃ (10.5 g, 11.45 mmol), x-phos(10.7 g, 22.9 mmol), K₃PO₄(69.4 g, 327.3 mmol), and 820 mL of toluene were added to a flask, and stirred at 140° C. for 2 hours. After completion of the reaction, the reactant was cooled to room temperature and separated by column chromatography to obtain Compound 2-1 (25 g, yield: 41%).

### 2) Synthesis of Compound 2-2

Compound 2-1 (25 g, 72.3 mmol), Cu(OTf)₂ (78.4 g, 216.9 mmol), and 900 mL of o-DCB were added to a flask, and stirred at 160° C. for 48 hours. After completion of the reaction, the mixture was distilled to concentrate the organic matter and separated by column chromatography to obtain Compound 2-2 (5.7 g, yield: 23%).

### 3) Synthesis of Compound C-63

Compound 2-2 (2.83 g, 8.23 mmol), N-phenyldibenzo[b,d]furan-3-amine (2.13 g, 8.23 mmol), Pd₂dba₃ (377 mg, 0.41 mmol), s-phos (337 mg, 0.82 mmol), NaOtBu (1.98 g, 20.58 mmol), and 55 mL of o-xylene were added to a flask and stirred at 150° C. for 4 hours. After completion of the reaction, the reactant was cooled to room temperature and separated by column chromatography to obtain Compound C-63 (1.9 g, yield: 41%).

| | MW | M.P. |
|---|---|---|
| C-63 | 566.62 | 284° C. |

### Example 6: Preparation of Compound C-125

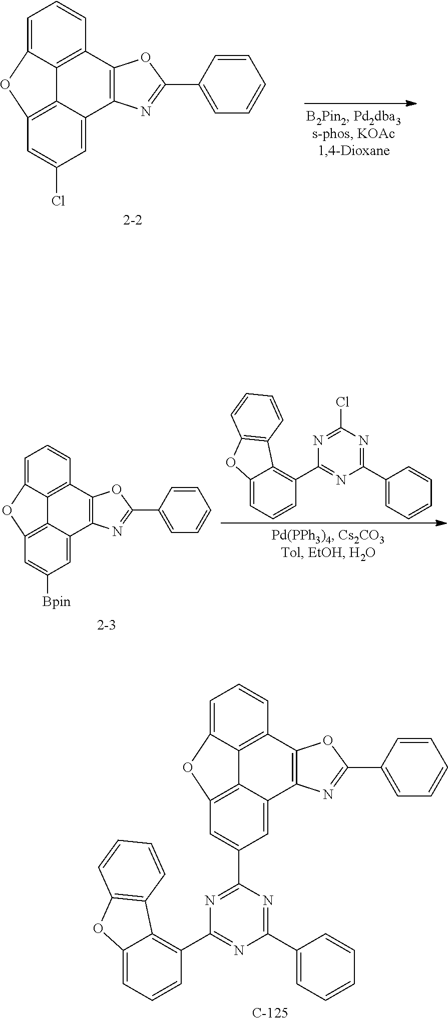

### 1) Synthesis of Compound 2-3

Compound 2-2 (2.0 g, 5.82 mmol), bis(pinacolato)diboron (2.91 g, 11.40 mmol), Pd₂dba₃ (520 mg, 0.57 mmol), s-phos (470 mg, 1.14 mmol), KOAc (1.7 g, 17.1 mmol), and 40 mL of 1,4-dioxane were added to a flask and stirred at 130° C. for 4 hours. After completion of the reaction, the reactant was cooled to room temperature and separated by column chromatography to obtain Compound 2-3 (2.2 g, yield: 87%).

### 2) Synthesis of Compound C-125

Compound 2-3 (2.2 g, 5.05 mmol), 2-chloro-4-(dibenzo[b,d]furan-1-yl)-6-phenyl-1,3,5-triazine (1.81 g, 5.05 mmol), Pd(PPh₃)₄ (290 mg, 0.25 mmol), Cs₂CO₃ (4.1 g, 12.63 mmol), 24 mL of toluene, 6 mL of ethanol, and 6 mL of H₂O were added to a flask, and stirred at 140° C. for 2 hours. After completion of the reaction, the reactant was cooled to room temperature and separated by column chromatography to obtain Compound C-125 (2.1 g, yield: 66%).

| | MW | M.P. |
|---|---|---|
| C-125 | 630.66 | 342° C. |

### Device Example 1: Producing an OLED Using Second Hole Transport Layer Compound According to the Present Disclosure

An OLED according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and was then stored in isopropyl alcohol. The ITO substrate was mounted on a substrate holder of a vacuum vapor deposition apparatus. Compound HI-1, shown in Table 4, was introduced into a cell of the vacuum vapor deposition apparatus, and compound HT-1 was introduced into another cell. The two materials were evaporated at different rates, and compound HI-1 was deposited in a doping amount of 3 wt % based to the total amount of compound HI-1 and compound HT-1 to form a first hole injection layer with a thickness of 10 nm. Subsequently, compound HT-1 was deposited on the first hole injection layer to form a first hole transport layer with a thickness of 80 nm. Next, compound C-15 was introduced into another cell of the vacuum vapor deposition apparatus and was evaporated by applying an electric current to the cell, thereby depositing a second hole transport layer with a thickness of 60 nm on the first hole transport layer. After forming the hole injection layer and the hole transport layers, a light-emitting layer was deposited thereon as follows: each of the first host compound H1 and the second host compound H2-76 were introduced into two cells of the vacuum vapor deposition apparatus as hosts, and compound D-39 was introduced into another cell as a dopant. The two host materials were evaporated at a rate of 1:1, the dopant material was simultaneously evaporated at a different rate, and the dopant was deposited in a doping amount of 3 wt % based on the total amount of the hosts and dopant to form a light-emitting layer with a thickness of 40 nm on the second hole transport layer. Then, compound ET-2 and compound EI-1 were evaporated at a weight ratio of 50:50 as an electron transport material to form an electron transport layer having a thickness of 35 nm on the light-emitting layer. After depositing compound EI-1 as an electron injection layer with a thickness of 2 nm on the electron transport layer, an Al cathode was deposited with a thickness of 80 nm on the electron injection layer by using another vacuum vapor deposition apparatus, thereby producing an OLED. All of the materials used for producing the OLED were purified by vacuum sublimation at 10⁻⁶ torr.

### Comparative Example 1: Producing an OLED Comprising a Comparative Compound as a Host

An OLED was produced in the same manner as in Device Example 1, except that the material in Table 1 below was used as a second hole transport layer material.

Table 1, below, shows the driving voltage, luminous efficiency, light-emitting color at a luminance of 10,000 nit, and time taken for luminance to reduce from 100% to 95% at a luminance of 10,000 nit (lifespan: T95) for the OLEDs of Device Example 1, and Comparative Example 1 produced as described above.

TABLE 1

| | Second hole | Driving | Luminous | Light- | |
|---|---|---|---|---|---|
| | transport | Voltage | Efficiency | Emitting | Lifespan |
| | layer | (V) | (cd/A) | Color | T95(hr) |
| Device | C-15 | 2.8 | 23.7 | Red | 51 |
| Example 1 | | | | | |
| Comparative | HT-Ref | 3.1 | 12.5 | Red | 9 |
| Example 1 | | | | | |

From Table 1 above, it can be confirmed that the OLED comprising the compound according to the present disclosure in the second hole transport layer (Device Example 1) exhibits significantly improved luminous efficiency and lifespan characteristics compared to the OLED not comprising the compound according to the present disclosure (Comparative Example 1).

### Device Example 2: Producing an OLED Comprising the Plurality of Host Materials According to the Present Disclosure

An OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 shown in Table 4 below was introduced into a cell of the vacuum vapor deposition apparatus, and Compound HT-1 was introduced into another cell of the vacuum vapor deposition apparatus. The two materials were evaporated at different rates, and Compound HI-1 was deposited in a doping amount of 3 wt % based on the total amount of Compound HI-1 and Compound HT-1 to form a first hole injection layer having a thickness of 10 nm on the ITO substrate. Next, Compound HT-1 was deposited on the first hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was deposited thereon as follows: each of the first host compound and the second host compound shown in Table 2 below were introduced into two cells of the vacuum vapor deposition apparatus as hosts, and compound D-39 was introduced into another cell as a dopant. The two host materials were evaporated at a rate of 1:1, the dopant material was simultaneously evaporated at a different rate, and the dopant was deposited in a doping amount of 3 wt % based on the total amount of the hosts and dopant to form a light-emitting layer with a thickness of 40 nm on the second hole transport layer. Then, compound ET-1 and compound EI-1 were evaporated at a weight ratio of 50:50 as an electron transport material to form an electron transport layer having a thickness of 35 nm on the light-emitting layer. After depositing compound EI-1 as an electron injection layer with a thickness of 2 nm on the electron transport layer, an Al cathode was deposited with a thickness of 80 nm on the electron injection layer by using another vacuum vapor deposition apparatus, thereby producing an OLED. All of the materials used for producing the OLED were purified by vacuum sublimation at 10⁻⁶ torr.

### Device Examples 3 to 5: Producing OLEDs Comprising the Plurality of Host Materials According to the Present Disclosure

In Device Examples 3 to 5, OLEDs were produced in the same manner as in Device Example 2, except that compounds C-15, C-13, and C-63 described in Table 2 below were used instead of compound C-10 as the first host compound.

### Comparative Example 2: Producing an OLED Comprising a Comparative Compound as a Host

An OLED was produced in the same manner as in Device Example 2, except that the second host compound of Table 2 below was used alone as a host for the light-emitting layer.

Table 2, below, shows the driving voltage, luminous efficiency, light-emitting color at a luminance of 1,000 nit, and time taken for luminance to reduce from 100% to 95% at a luminance of 10,000 nit (lifespan: T95) for the OLEDs of Device Examples 2 to 5, and Comparative Example 2 produced as described above.

TABLE 2

| | | | Driving | Luminous | Light- | |
|---|---|---|---|---|---|---|
| | First | Second | Voltage | Efficiency | Emitting | Lifespan |
| | Host | Host | (V) | (cd/A) | Color | T95(hr) |
| Device | C-10 | H2-76 | 2.9 | 27.2 | Red | 117 |
| Example 2 | | | | | | |
| Device | C-15 | H2-76 | 2.9 | 27.6 | Red | 210 |
| Example 3 | | | | | | |
| Device | C-13 | H2-76 | 2.9 | 25.7 | Red | 127 |
| Example 4 | | | | | | |
| Device | C-63 | H2-76 | 2.9 | 28.3 | Red | 52 |
| Example 5 | | | | | | |
| Comparative | — | H2-76 | 3.0 | 24.6 | Red | 15 |
| Example 2 | | | | | | |

From Table 2 above, it can be confirmed that OLEDs comprising the plurality of host materials according to the present disclosure (Device Examples 2 to 5) exhibit a driving voltage and luminous efficiency characteristics at an equivalent level, as well as significantly improved lifetime properties, compared to the OLED comprising a single host material (Comparative Example 2).

### Device Example 6: Producing an OLED Comprising the Single Host Compound According to the Present Disclosure

An OLED according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and was then stored in isopropyl alcohol. The ITO substrate was mounted on a substrate holder of a vacuum vapor deposition apparatus. Compound HI-1, shown in Table 4, was introduced into a cell of the vacuum vapor deposition apparatus, and compound HT-3 was introduced into another cell. The two materials were evaporated at different rates, and compound HI-1 was deposited in a doping amount of 3 wt % based to the total amount of compound HI-1 and compound HT-3 to form a first hole injection layer with a thickness of 10 nm. Subsequently, compound HT-3 was deposited on the first hole injection layer to form a first hole transport layer with a thickness of 80 nm. Next, compound HT-4 was introduced into another cell of the vacuum vapor deposition apparatus and was evaporated by applying an electric current to the cell, thereby depositing a second hole transport layer with a thickness of 55 nm on the first hole transport layer. Next, compound HT-5 was introduced into another cell of the vacuum vapor deposition apparatus and was evaporated by applying an electric current to the cell, thereby depositing a third hole transport layer with a thickness of 5 nm on the second hole transport layer. After forming the hole injection layer and the hole transport layers, a light-emitting layer was deposited thereon as follows: a host compound shown in Table 3 below were introduced into a cell of the vacuum vapor deposition apparatus as a host, and compound D-39 was introduced into another cell as a dopant. The host material and dopant material were evaporated at a different rate, and the dopant was deposited in a doping amount of 3 wt % based on the total amount of the host and dopant to form a light-emitting layer with a thickness of 40 nm on the third hole transport layer. Then, compound ET-2 and compound EI-1 were evaporated at a weight ratio of 50:50 as an electron transport material to form an electron transport layer having a thickness of 35 nm on the light-emitting layer. After depositing compound EI-1 as an electron injection layer with a thickness of 2 nm on the electron transport layer, an Al cathode was deposited with a thickness of 80 nm on the electron injection layer by using another vacuum vapor deposition apparatus, thereby producing an OLED. All of the materials used for producing the OLED were purified by vacuum sublimation at 10⁻⁶ torr.

### Comparative Example 3: Producing an OLED Comprising a Comparative Compound as a Host

An OLED was produced in the same manner as in Device Example 6, except that the host compound of Table 3 below was used as a host for the light-emitting layer.

Table 3, below, shows the driving voltage, luminous efficiency, light-emitting color at a luminance of 1,000 nit, and time taken for luminance to reduce from 100% to 95% at a luminance of 10,000 nit (lifespan: T95) for the OLED of Device Example 6, and Comparative Example 3 produced as described above.

TABLE 3

| | | Driving | Luminous | Light- | |
|---|---|---|---|---|---|
| | | Voltage | Efficiency | Emitting | Lifespan |
| | Host | (V) | (cd/A) | Color | T95(hr) |
| Device | C-125 | 3.2 | 24.6 | Red | 12.2 |
| Example 6 | | | | | |
| Comparative | H2-81 | 3.8 | 6.2 | Red | 0.2 |
| Example 3 | | | | | |

From Table 3 above, it can be confirmed that the OLED comprising the compound according to the present disclosure in the light-emitting layer as a host (Device Example 6) exhibits lower driving voltage, and significantly improved luminous efficiency and lifetime properties, compared to the OLED not comprising the compound according to the present disclosure (Comparative Example 3).

The compounds used in the Device Examples and the Comparative Examples are shown in Table 4 below.

TABLE 4

| | |
|---|---|
| Hole Injection Layer/ Hole Transport Layer | 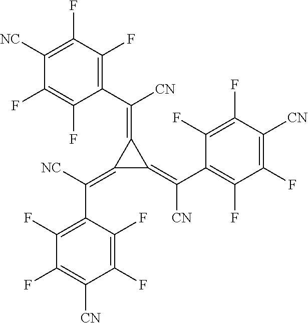 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 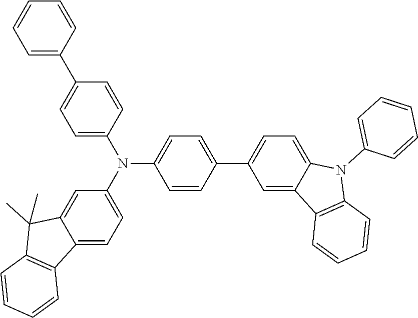 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] |
| | HT-1 |
| | 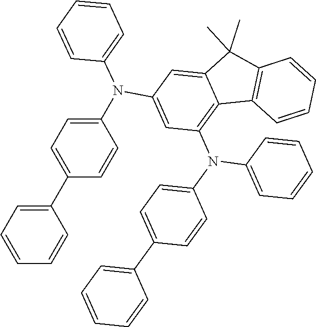 [SMILES: CC1(C)c2ccccc2-c2c(N(c3ccccc3)c3ccc(-c4ccccc4)cc3)cc(N(c3ccccc3)c3ccc(-c4ccccc4)cc3)cc21] |
| | HT-2 |
| | 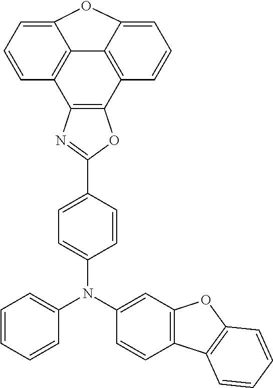 [SMILES: c1ccc(N(c2ccc(-c3nc4c5cccc6oc7cccc(c4o3)c7c65)cc2)c2ccc3c(c2)oc2ccccc23)cc1] |
| | C-15 |
| | 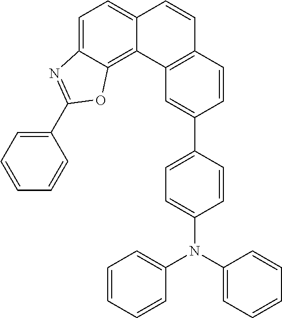 [SMILES: c1ccc(-c2nc3ccc4ccc5ccc(-c6ccc(N(c7ccccc7)c7ccccc7)cc6)cc5c4c3o2)cc1] |
| | HT-Ref |
| | 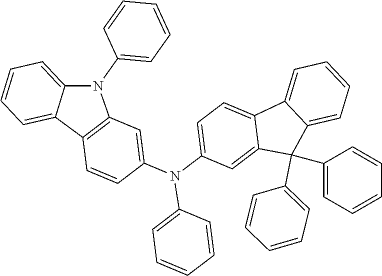 [SMILES: c1ccc(N(c2ccc3c(c2)C(c2ccccc2)(c2ccccc2)c2ccccc2-3)c2ccc3c4ccccc4n(-c4ccccc4)c3c2)cc1] |
| | HT-3 |
| | 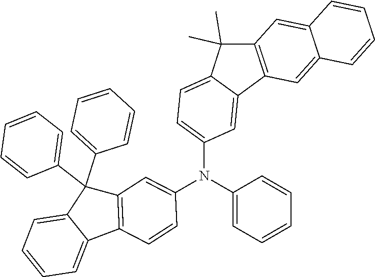 [SMILES: CC1(C)c2ccc(N(c3ccccc3)c3ccc4c(c3)C(c3ccccc3)(c3ccccc3)c3ccccc3-4)cc2-c2cc3ccccc3cc21] |
| | HT-4 |
| | 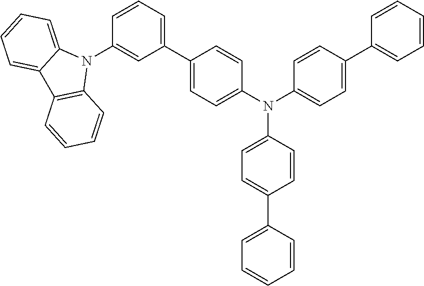 [SMILES: c1ccc(-c2ccc(N(c3ccc(-c4ccccc4)cc3)c3ccc(-c4cccc(-n5c6ccccc6c6ccccc65)c4)cc3)cc2)cc1] |
| | HT-5 |
| Light-Emitting Layer | 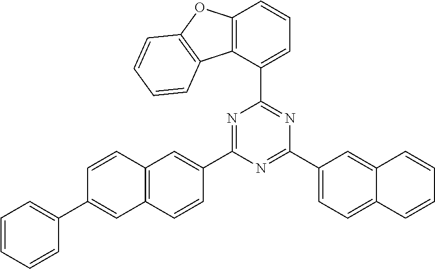 [SMILES: c1ccc(-c2ccc3cc(-c4nc(-c5ccc6ccccc6c5)nc(-c5cccc6oc7ccccc7c56)n4)ccc3c2)cc1] |
| | H2-76 |
| | 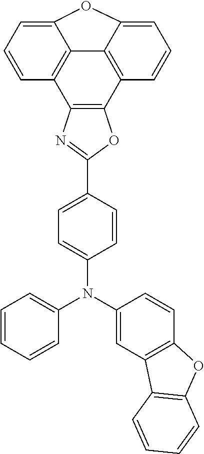 [SMILES: c1ccc(N(c2ccc(-c3nc4c5cccc6oc7cccc(c4o3)c7c65)cc2)c2ccc3oc4ccccc4c3c2)cc1] |
| | C-10 |
| | 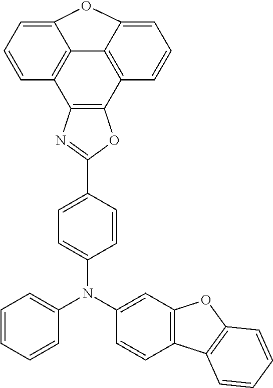 [SMILES: c1ccc(N(c2ccc(-c3nc4c5cccc6oc7cccc(c4o3)c7c65)cc2)c2ccc3c(c2)oc2ccccc23)cc1] |
| | C-15 |
| | 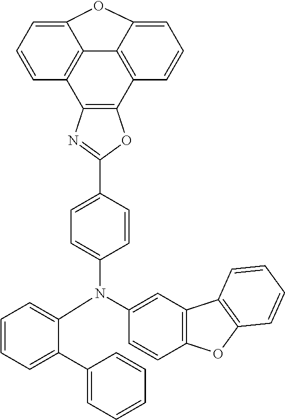 [SMILES: c1ccc(-c2ccccc2N(c2ccc(-c3nc4c5cccc6oc7cccc(c4o3)c7c65)cc2)c2ccc3oc4ccccc4c3c2)cc1] |
| | C-13 |
| | 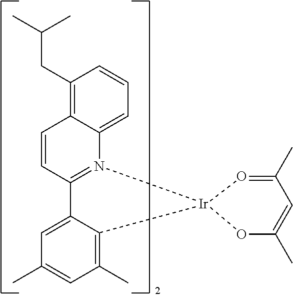 |
| | D-39 |
| | 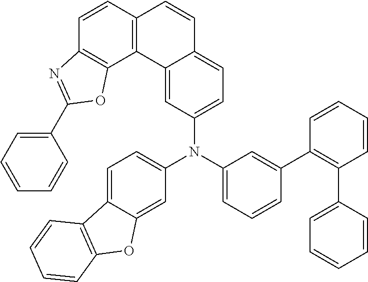 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1] |
| | H1 |
| | 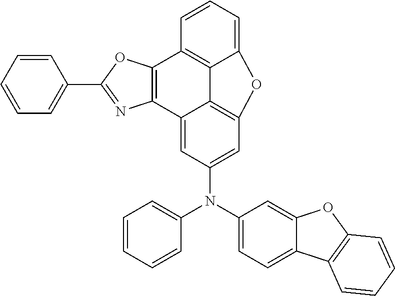 [SMILES: c1ccc(-c2nc3c4cc(N(c5ccccc5)c5ccc6c(c5)oc5ccccc56)cc5oc6cccc(c3o2)c6c54)cc1] |
| | C-63 |
| | 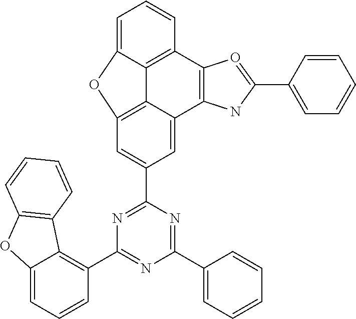 |
| | C-125 |
| | 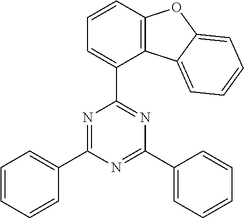 [SMILES: c1ccc(-c2nc(-c3ccccc3)nc(-c3cccc4oc5ccccc5c34)n2)cc1] |
| | H2-81 |
| Electron Transport Layer/ Electron Injection Layer | 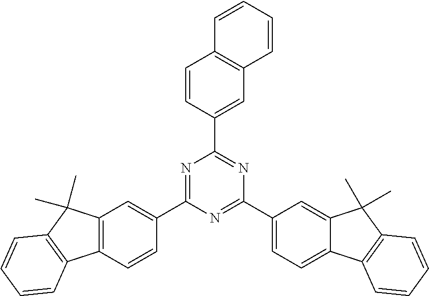 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | ET-1 |
| | 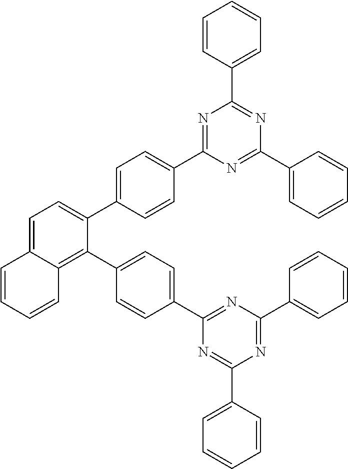 [SMILES: c1ccc(-c2nc(-c3ccccc3)nc(-c3ccc(-c4ccc5ccccc5c4-c4ccc(-c5nc(-c6ccccc6)nc(-c6ccccc6)n5)cc4)cc3)n2)cc1] |
| | ET-2 |
| | 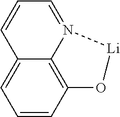 |
| | EI-1 |

1. An organic electroluminescent compound represented by the following formula 1: 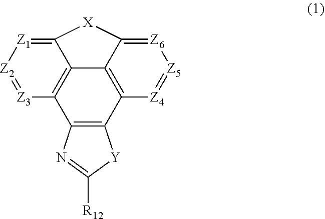
in formula 1,
X represents NR₁, O, S, Se, CR₂R₃, or SiR₄R₅;
R₁ to R₅ each independently represent a substituted or unsubstituted (C1-C30)alkyl, a substituted or unsubstituted (C6-C30)aryl, or a substituted or unsubstituted (3- to 30-membered)heteroaryl;
Y represents O, S, Se, or Te;
Z₁ to Z₆ each independently represent N or CR₁₁;
R₁₁ represents hydrogen, deuterium, or -L₁—R₁₃;
R₁₂ represents -L₂-R₁₄;
L₁ and L₂ each independently represent a single bond, a substituted or unsubstituted (C6-C30)arylene, or a substituted or unsubstituted (3- to 30-membered)heteroarylene;
R₁₃ and R₁₄ each independently represent a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, -L′₁—SiR′₁R′₂R′₃, -L′₂—GeR′₄R′₅R′₆, -L′₃—NR′₇R′₈, -L′₄(NR′₉R′₁₀)(NR′₁₁R′₁₂), -L′₅—NR′₁₃R′₁₄, or -L′₆—(NR′₁₅)—L′₇(NR′₁₆R′₁₇);
L′₁ to L′₇ each independently represent a single bond, a substituted or unsubstituted (C6-C30)arylene, or a substituted or unsubstituted (3- to 30-membered)heteroarylene; and
R′₁ to R′₁₇ each independently represent a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl.

2. The organic electroluminescent compound according to claim 1, wherein formula 1 is represented by one of the following formulas 1-1 to 1-3: 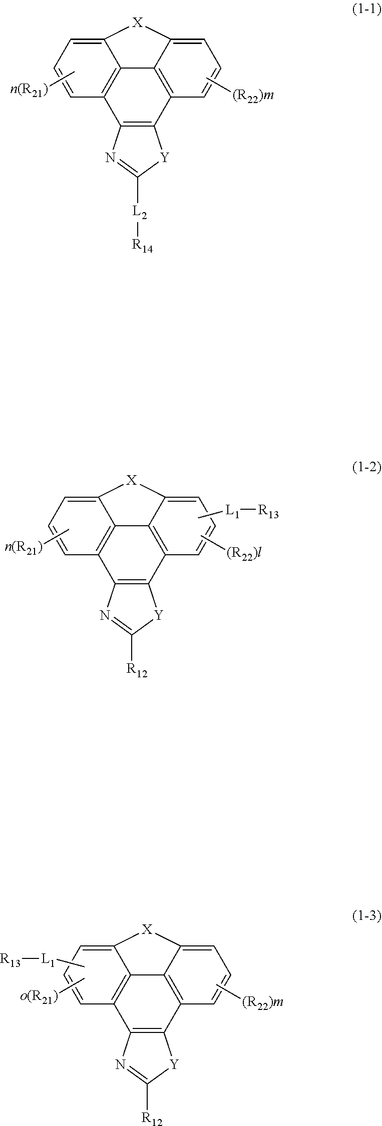
in formulas,
R₂₁ and R₂₂ each independently represent hydrogen, deuterium, or a (C6-C30)aryl unsubstituted or substituted with deuterium;
n and m each independently represent an integer of 3, I and o each independently represent an integer of 2, and each of R₂₁ and each of R₂₂ may be the same as or different from each other; and
L₁, L₂, X, Y, and R₁₂ to R₁₄ are as defined in claim 1.

3. The organic electroluminescent compound according to claim 1, wherein X and Y each independently represent O or S.

4. The organic electroluminescent compound according to claim 1, wherein formula 1 is represented by one of the following formulas 1-11 to 1-21: 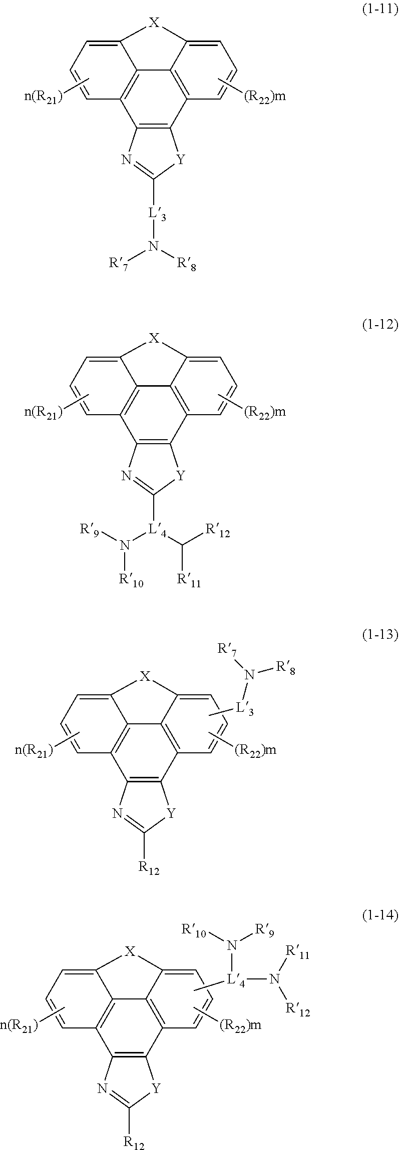 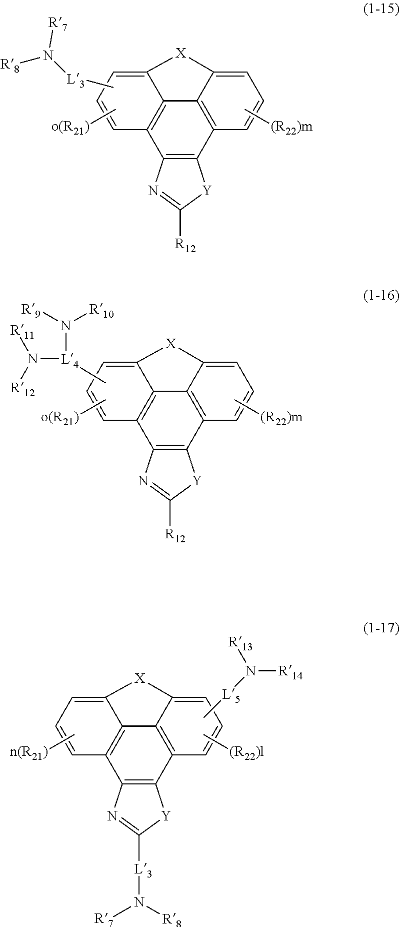 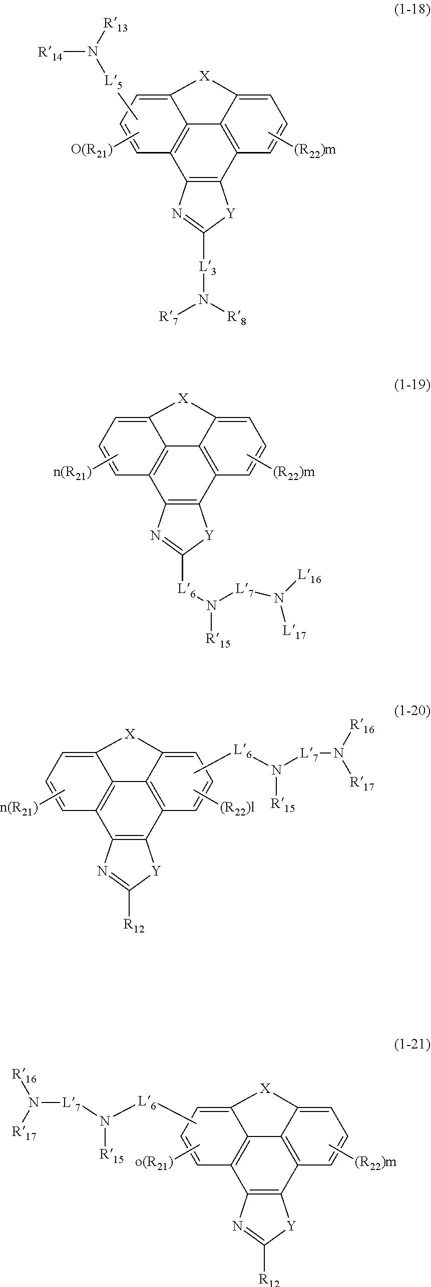
in formulas,
R₂₁ and R₂₂ each independently represent hydrogen, deuterium, or a (C6-C30)aryl unsubstituted or substituted with deuterium;
n and m each independently represent an integer of 3, I and o each independently represent an integer of 2, and each of R₂₁ and each of R₂₂ may be the same as or different from each other; and
X, Y, L′₃ to L′₇, R′₇ to R′₁₇, and R₁₂ are as defined in claim 1.

5. The organic electroluminescent compound according to claim 4, wherein R′₇ to R′₁₇ each independently are selected from a phenyl, a biphenyl, a terphenyl, a quinquiphenyl, a naphthyl, a binaphthyl, a phenylnaphthyl, a naphthylphenyl, a chrysenyl, a fluorenyl, a dimethylfluorenyl, a diethylfluorenyl, a methylphenylfluorenyl, a diphenylfluorenyl, a phenanthrenyl, a dibenzofuranyl, a dibenzothiophenyl, a pyridyl, a carbazolyl, or a combination thereof, and the selected substituents may be partially or fully substituted with deuterium.

6. The organic electroluminescent compound according to claim 1, wherein formula 1 is represented by one of the following formulas 1-31 to 1-33: 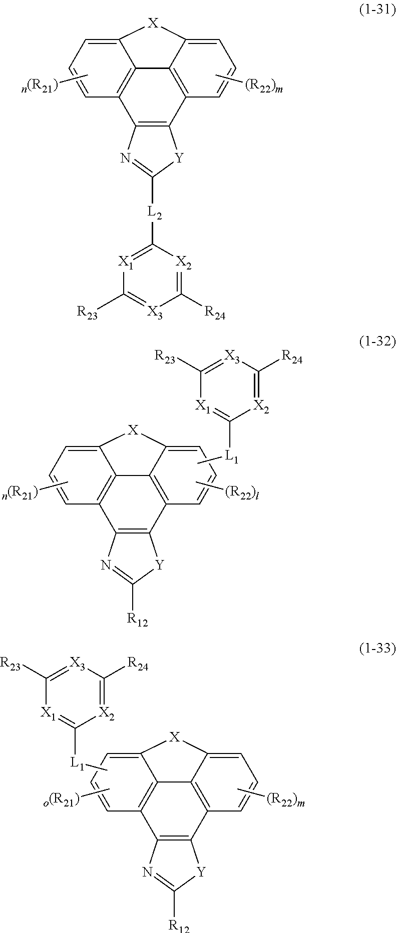
in formulas,
X₁ to X₃ each independently represent N, CH, or CD, and at least one of these is N;
R₂₃ and R₂₄ each independently represent a substituted or unsubstituted (C6-C30)aryl, or a substituted or unsubstituted (3- to 30-membered)heteroaryl;
R₂₁ and R₂₂ each independently represent hydrogen, deuterium, or a (C6-C30)aryl;
n and m each independently represent an integer of 3, I and o each independently represent an integer of 2, and each of R₂₁ and each of R₂₂ may be the same as, or different from each other; and
X, Y, L₁, L₂, and R₁₂ are as defined in claim 1.

7. The organic electroluminescent compound according to claim 6, wherein R₂₃ and R₂₄ each independently are selected from a phenyl, a biphenyl, a terphenyl, a quinquiphenyl, a naphthyl, a binaphthyl, a phenylnaphthyl, a naphthylphenyl, a chrysenyl, a fluorenyl, a dimethylfluorenyl, a methylphenylfluorenyl, a diphenylfluorenyl, a phenanthrenyl, a dibenzofuranyl, a dibenzothiophenyl, a pyridyl, a carbazolyl, or a combination thereof, and the selected substituents may be partially or fully substituted with deuterium.

8. The organic electroluminescent compound according to claim 1, wherein the substituent(s) of the substituted alkyl, the substituted aryl, the substituted heteroaryl, the substituted arylene, the substituted heteroarylene, and the substituted cycloalkyl each independently are at least one selected from the group consisting of deuterium; a halogen; a cyano; a carboxyl; a nitro; a hydroxy; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of a (C6-C30)aryl(s); a (C6-C30)aryl unsubstituted or substituted with at least one of a (C1-C30)alkyl(s) and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C2-C30)alkenylamino; a mono- or di-(C6-C30)arylamino unsubstituted or substituted with a (C1-C30)alkyl(s); a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

9. The organic electroluminescent compound according to claim 1, wherein the organic electroluminescent compound represented by formula 1 is selected from the following compounds.

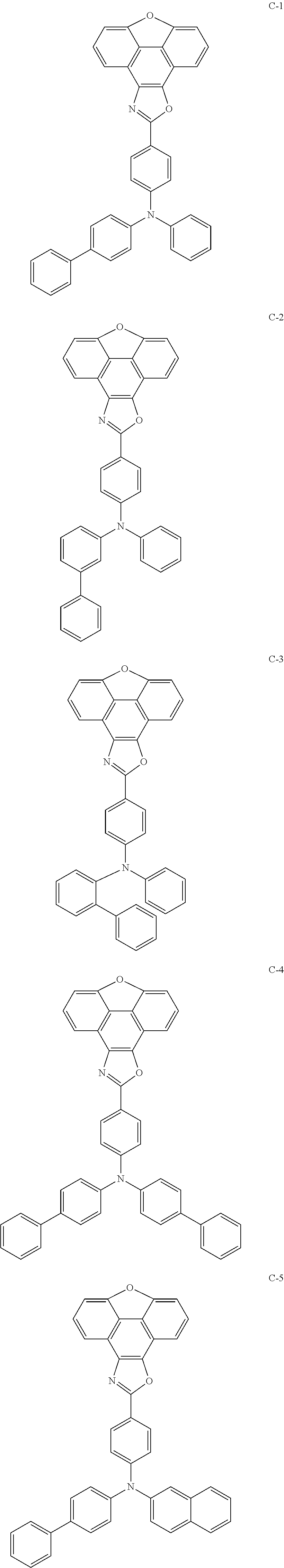 [SMILES: c1ccc(-c2ccc(N(c3ccccc3)c3ccc(-c4nc5c6cccc7oc8cccc(c5o4)c8c76)cc3)cc2)cc1 ; c1ccc(-c2cccc(N(c3ccccc3)c3ccc(-c4nc5c6cccc7oc8cccc(c5o4)c8c76)cc3)c2)cc1 ; c1ccc(-c2ccccc2N(c2ccccc2)c2ccc(-c3nc4c5cccc6oc7cccc(c4o3)c7c65)cc2)cc1 ; c1ccc(-c2ccc(N(c3ccc(-c4ccccc4)cc3)c3ccc(-c4nc5c6cccc7oc8cccc(c5o4)c8c76)cc3)cc2)cc1 ; c1ccc(-c2ccc(N(c3ccc(-c4nc5c6cccc7oc8cccc(c5o4)c8c76)cc3)c3ccc4ccccc4c3)cc2)cc1]

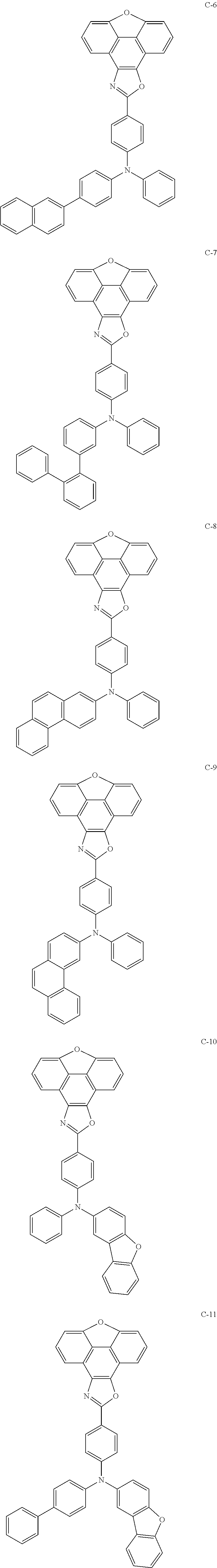 [SMILES: c1ccc(N(c2ccc(-c3ccc4ccccc4c3)cc2)c2ccc(-c3nc4c5cccc6oc7cccc(c4o3)c7c65)cc2)cc1 ; c1ccc(-c2ccccc2-c2cccc(N(c3ccccc3)c3ccc(-c4nc5c6cccc7oc8cccc(c5o4)c8c76)cc3)c2)cc1 ; c1ccc(N(c2ccc(-c3nc4c5cccc6oc7cccc(c4o3)c7c65)cc2)c2ccc3c(ccc4ccccc43)c2)cc1 ; c1ccc(N(c2ccc(-c3nc4c5cccc6oc7cccc(c4o3)c7c65)cc2)c2ccc3ccc4ccccc4c3c2)cc1 ; c1ccc(N(c2ccc(-c3nc4c5cccc6oc7cccc(c4o3)c7c65)cc2)c2ccc3oc4ccccc4c3c2)cc1 ; c1ccc(-c2ccc(N(c3ccc(-c4nc5c6cccc7oc8cccc(c5o4)c8c76)cc3)c3ccc4oc5ccccc5c4c3)cc2)cc1]

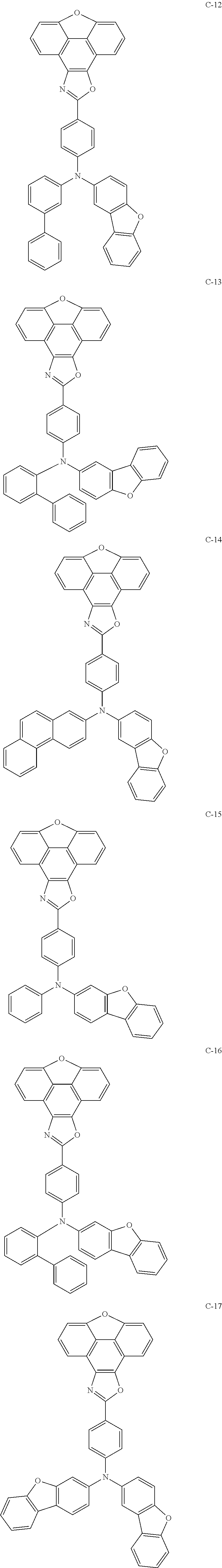

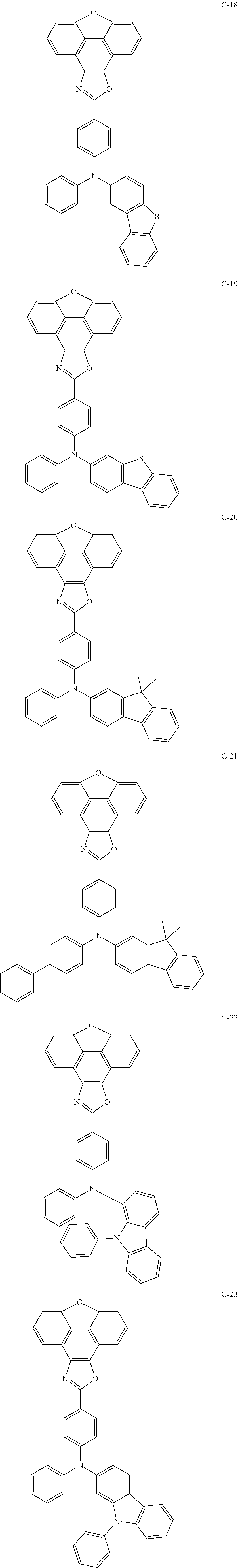 [SMILES: c1ccc(N(c2ccc(-c3nc4c5cccc6oc7cccc(c4o3)c7c65)cc2)c2ccc3sc4ccccc4c3c2)cc1 ; c1ccc(N(c2ccc(-c3nc4c5cccc6oc7cccc(c4o3)c7c65)cc2)c2ccc3c(c2)sc2ccccc23)cc1 ; CC1(C)c2ccccc2-c2ccc(N(c3ccccc3)c3ccc(-c4nc5c6cccc7oc8cccc(c5o4)c8c76)cc3)cc21 ; CC1(C)c2ccccc2-c2ccc(N(c3ccc(-c4ccccc4)cc3)c3ccc(-c4nc5c6cccc7oc8cccc(c5o4)c8c76)cc3)cc21 ; c1ccc(N(c2ccc(-c3nc4c5cccc6oc7cccc(c4o3)c7c65)cc2)c2cccc3c4ccccc4n(-c4ccccc4)c23)cc1 ; c1ccc(N(c2ccc(-c3nc4c5cccc6oc7cccc(c4o3)c7c65)cc2)c2ccc3c4ccccc4n(-c4ccccc4)c3c2)cc1]

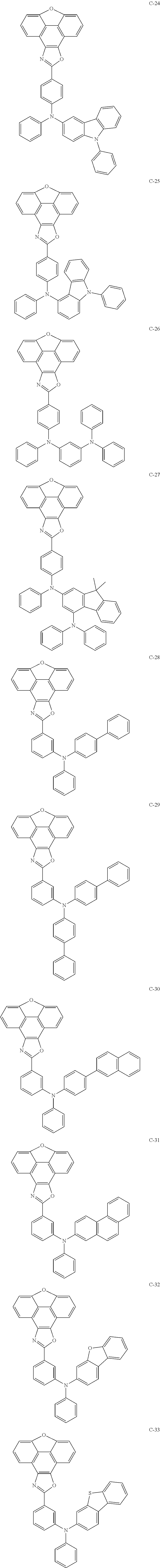

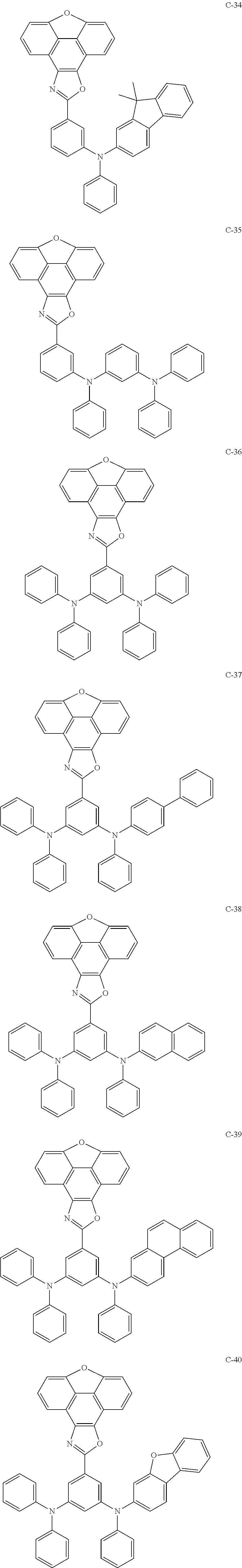

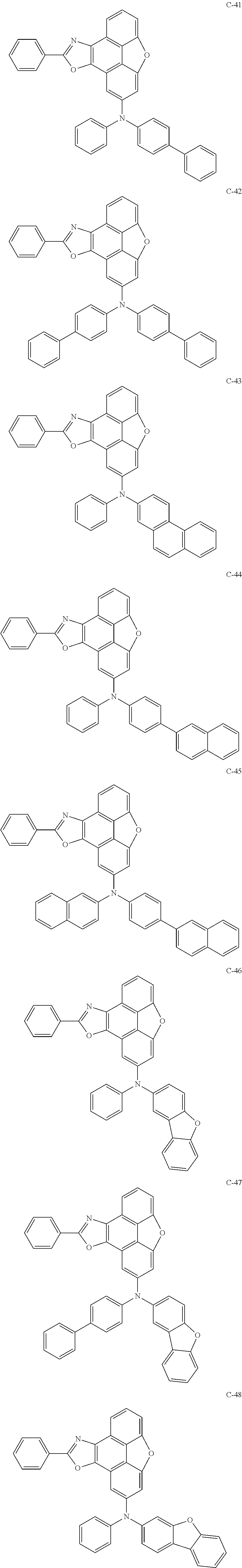

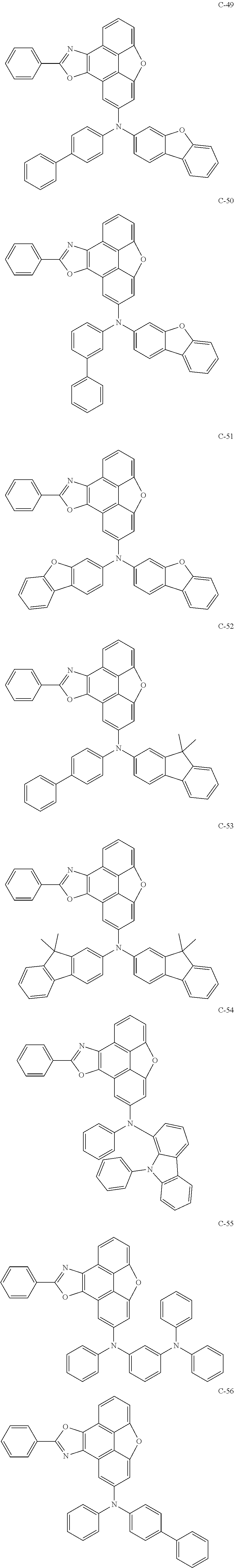

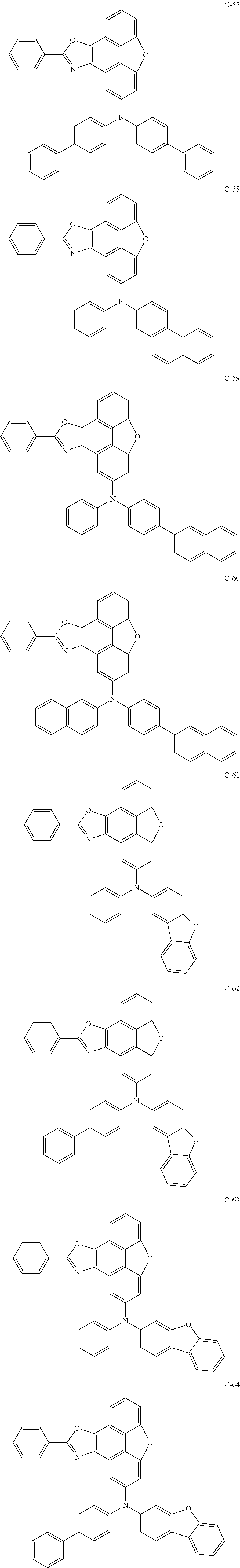

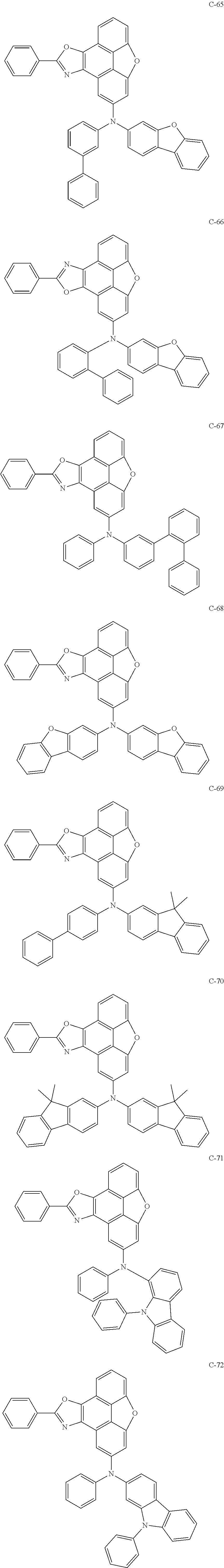

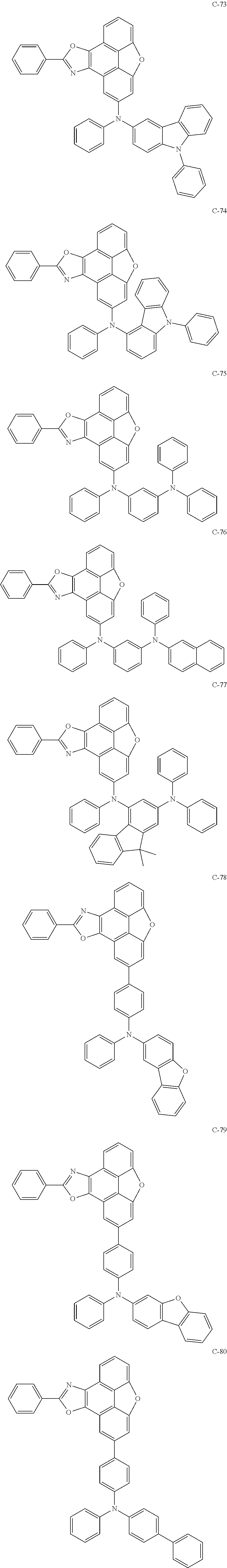

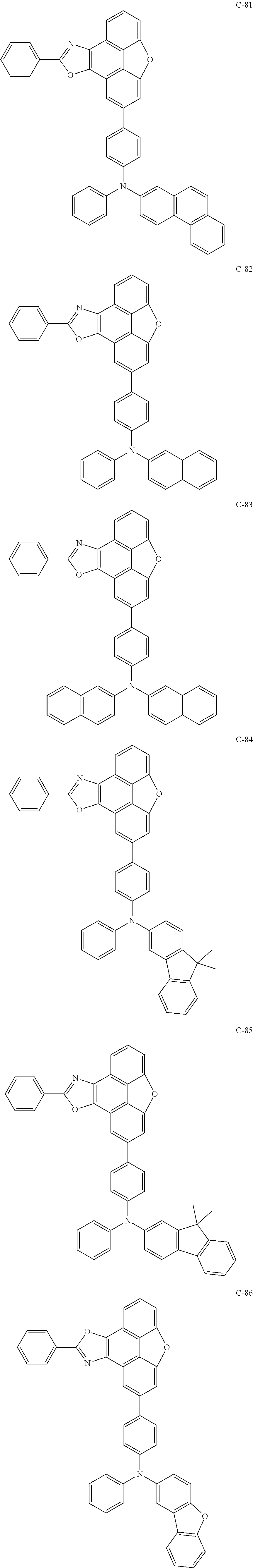

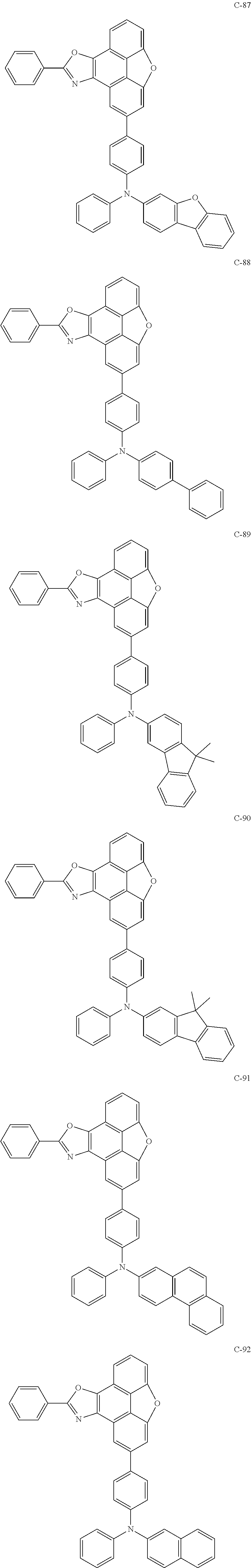

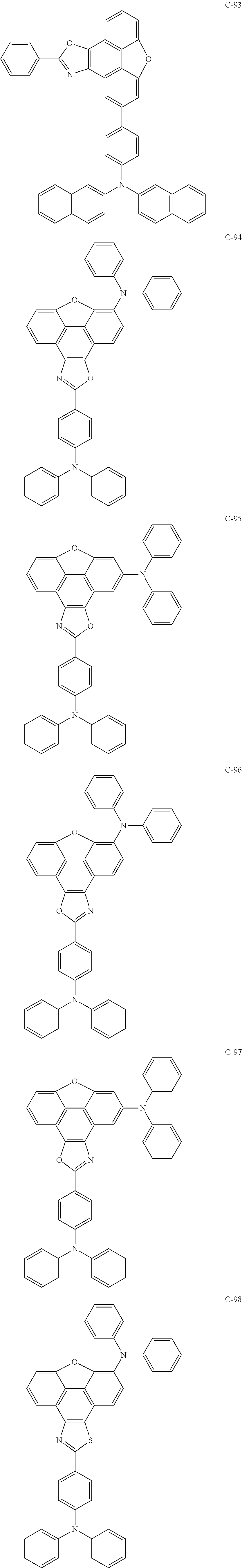

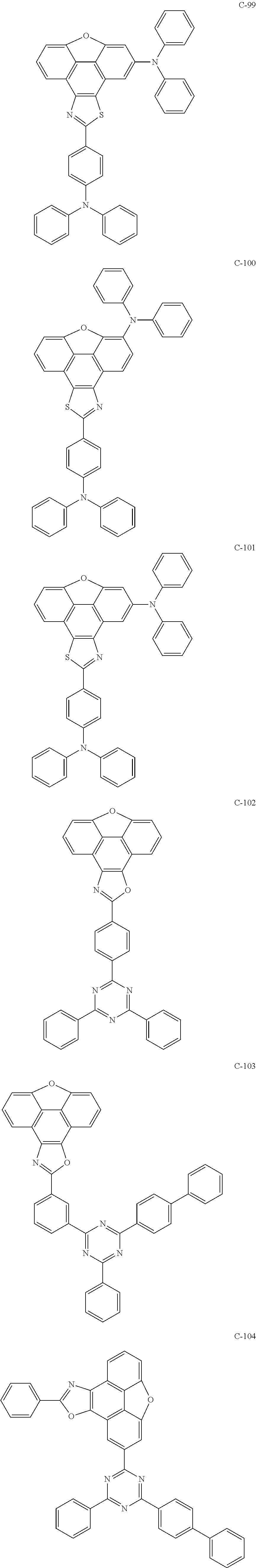

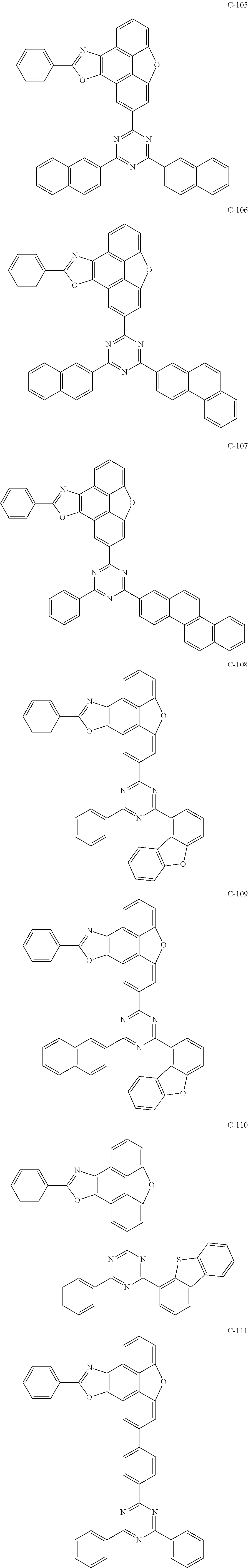

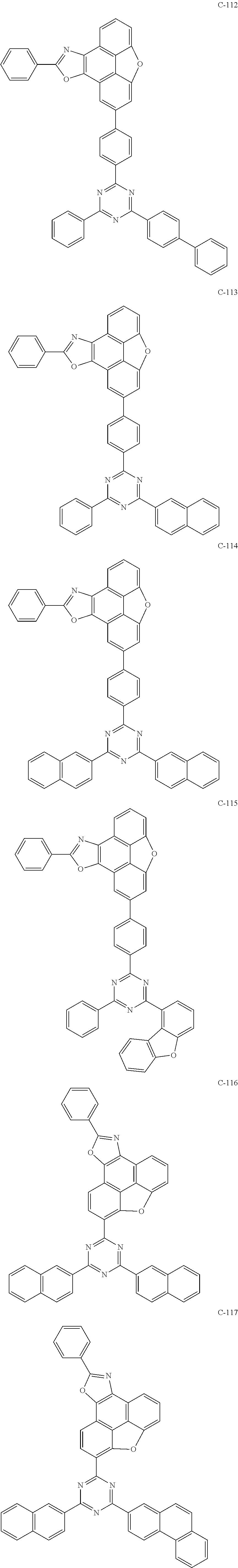

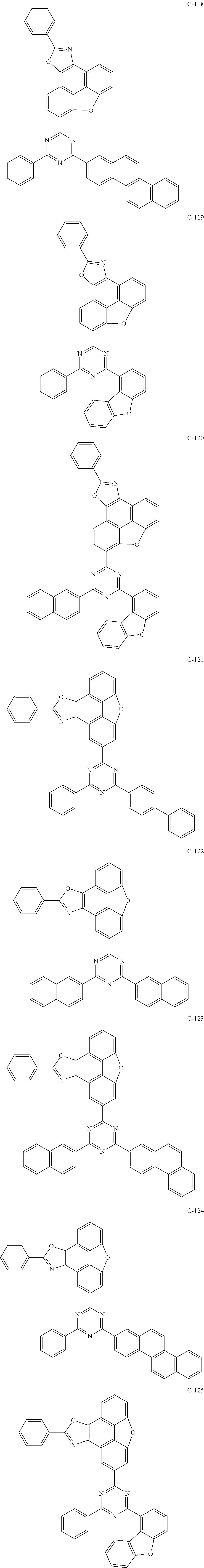

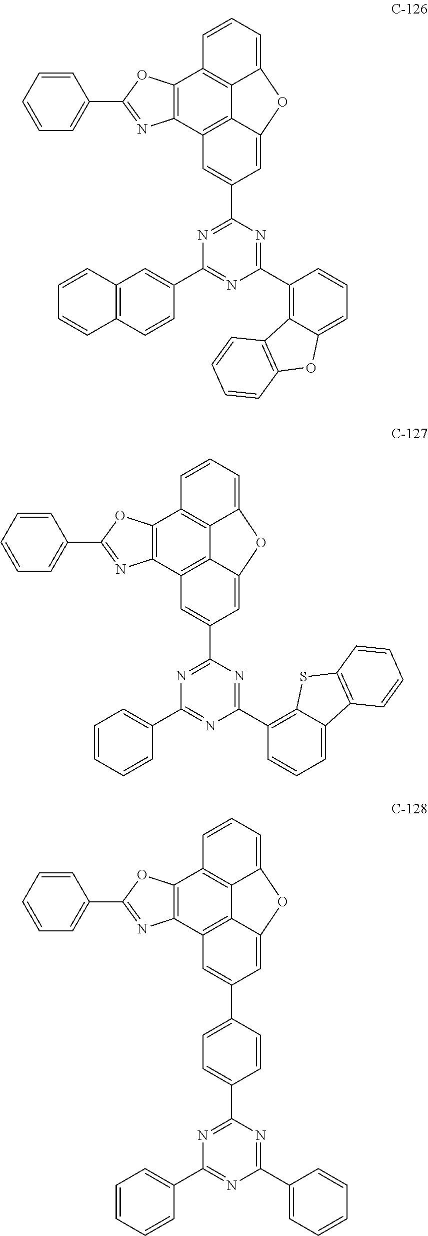 [SMILES: c1ccc(-c2nc3c4cc(-c5nc(-c6ccc7ccccc7c6)nc(-c6cccc7oc8ccccc8c67)n5)cc5oc6cccc(c3o2)c6c54)cc1 ; c1ccc(-c2nc(-c3cc4oc5cccc6c7oc(-c8ccccc8)nc7c(c3)c4c56)nc(-c3cccc4c3sc3ccccc34)n2)cc1 ; c1ccc(-c2nc(-c3ccccc3)nc(-c3ccc(-c4cc5oc6cccc7c8oc(-c9ccccc9)nc8c(c4)c5c67)cc3)n2)cc1]

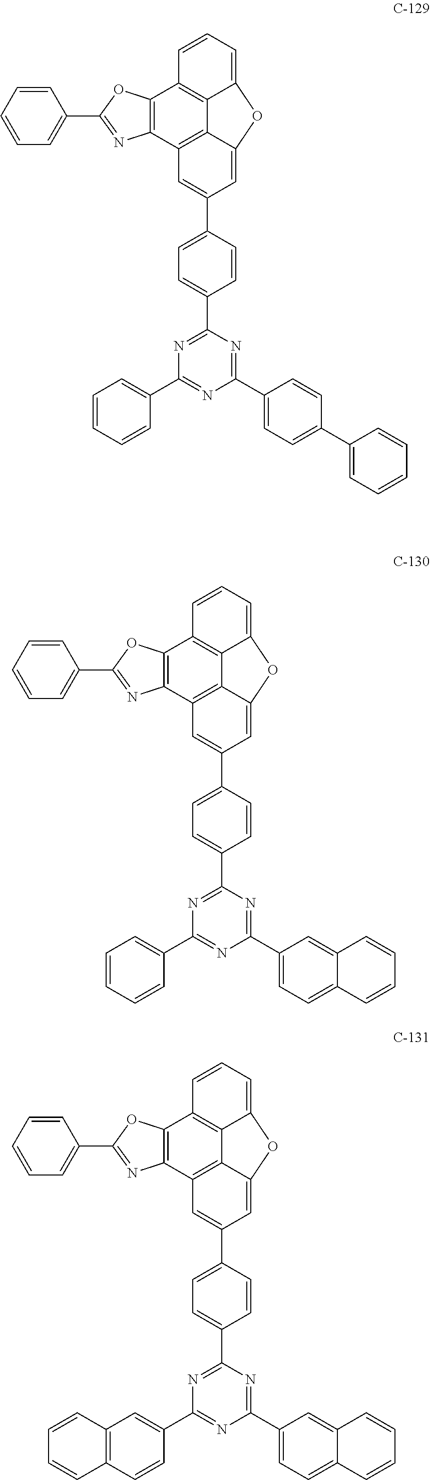 [SMILES: c1ccc(-c2ccc(-c3nc(-c4ccccc4)nc(-c4ccc(-c5cc6oc7cccc8c9oc(-c%10ccccc%10)nc9c(c5)c6c78)cc4)n3)cc2)cc1 ; c1ccc(-c2nc(-c3ccc(-c4cc5oc6cccc7c8oc(-c9ccccc9)nc8c(c4)c5c67)cc3)nc(-c3ccc4ccccc4c3)n2)cc1 ; c1ccc(-c2nc3c4cc(-c5ccc(-c6nc(-c7ccc8ccccc8c7)nc(-c7ccc8ccccc8c7)n6)cc5)cc5oc6cccc(c3o2)c6c54)cc1]

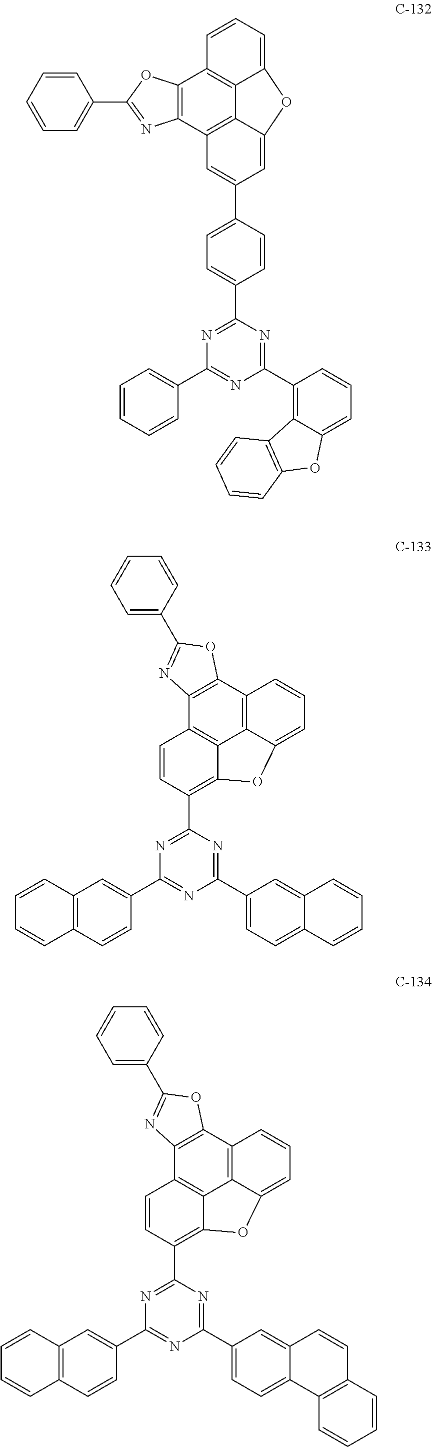 [SMILES: c1ccc(-c2nc(-c3ccc(-c4cc5oc6cccc7c8oc(-c9ccccc9)nc8c(c4)c5c67)cc3)nc(-c3cccc4oc5ccccc5c34)n2)cc1 ; c1ccc(-c2nc3c4ccc(-c5nc(-c6ccc7ccccc7c6)nc(-c6ccc7ccccc7c6)n5)c5oc6cccc(c3o2)c6c54)cc1 ; c1ccc(-c2nc3c4ccc(-c5nc(-c6ccc7ccccc7c6)nc(-c6ccc7c(ccc8ccccc87)c6)n5)c5oc6cccc(c3o2)c6c54)cc1]

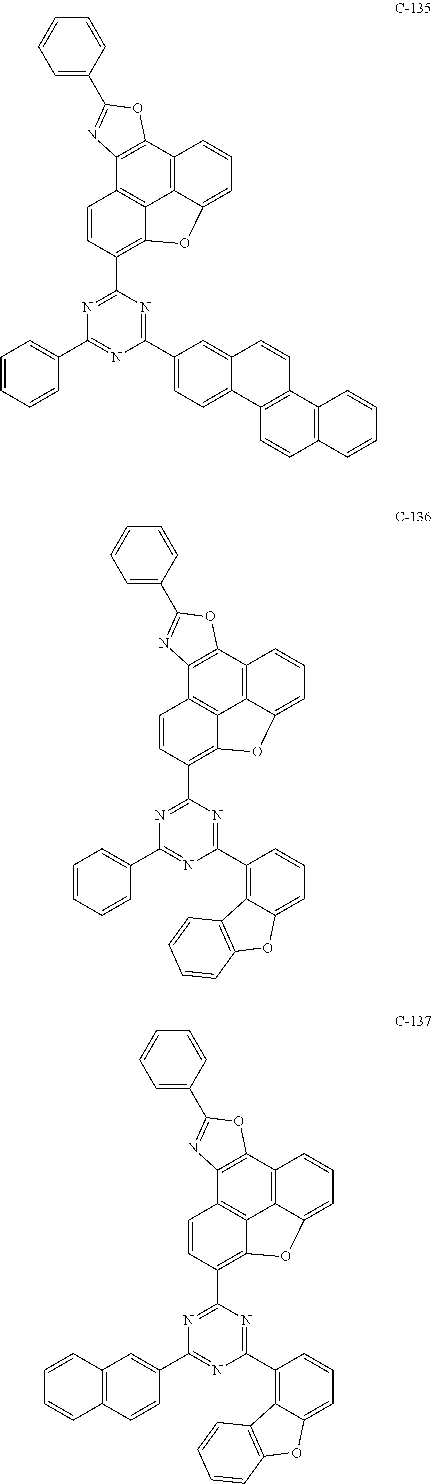 [SMILES: c1ccc(-c2nc(-c3ccc4c(ccc5c6ccccc6ccc45)c3)nc(-c3ccc4c5nc(-c6ccccc6)oc5c5cccc6oc3c4c65)n2)cc1 ; c1ccc(-c2nc(-c3ccc4c5nc(-c6ccccc6)oc5c5cccc6oc3c4c65)nc(-c3cccc4oc5ccccc5c34)n2)cc1 ; c1ccc(-c2nc3c4ccc(-c5nc(-c6ccc7ccccc7c6)nc(-c6cccc7oc8ccccc8c67)n5)c5oc6cccc(c3o2)c6c54)cc1]

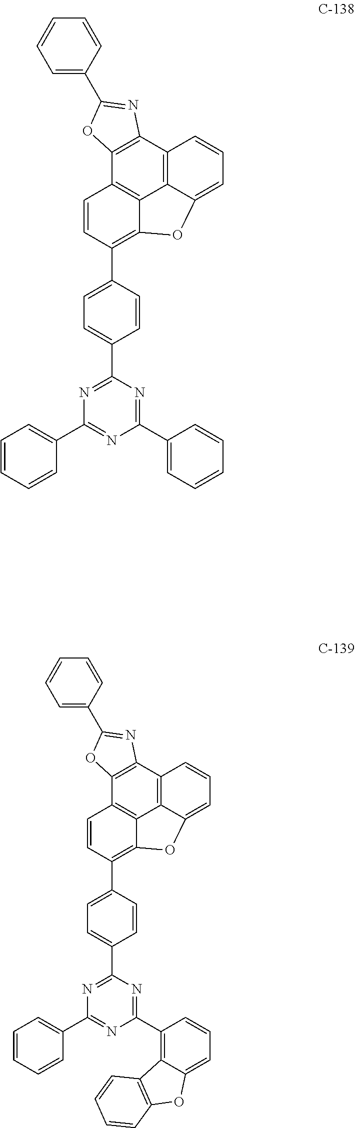 [SMILES: c1ccc(-c2nc(-c3ccccc3)nc(-c3ccc(-c4ccc5c6oc(-c7ccccc7)nc6c6cccc7oc4c5c76)cc3)n2)cc1 ; c1ccc(-c2nc(-c3ccc(-c4ccc5c6oc(-c7ccccc7)nc6c6cccc7oc4c5c76)cc3)nc(-c3cccc4oc5ccccc5c34)n2)cc1]

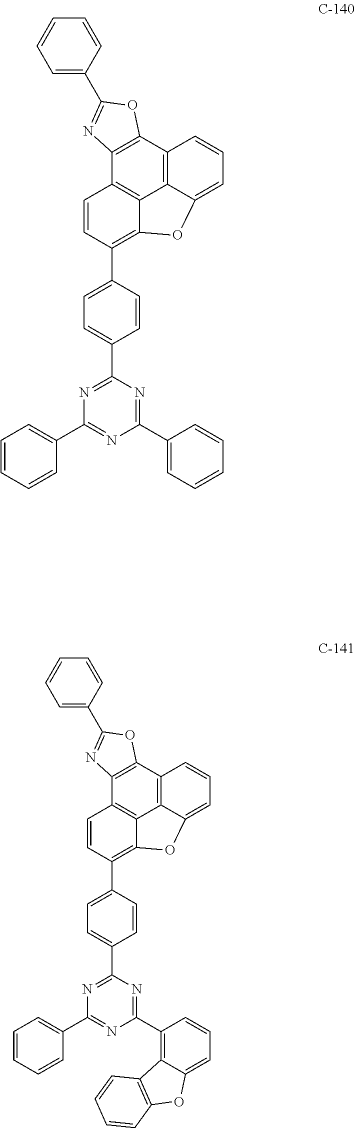 [SMILES: c1ccc(-c2nc(-c3ccccc3)nc(-c3ccc(-c4ccc5c6nc(-c7ccccc7)oc6c6cccc7oc4c5c76)cc3)n2)cc1 ; c1ccc(-c2nc(-c3ccc(-c4ccc5c6nc(-c7ccccc7)oc6c6cccc7oc4c5c76)cc3)nc(-c3cccc4oc5ccccc5c34)n2)cc1]

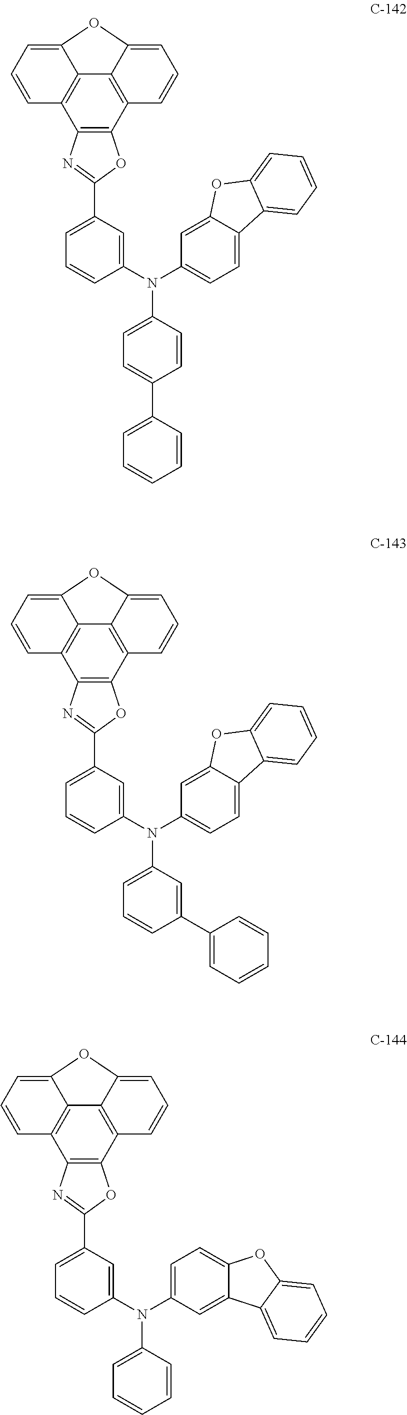 [SMILES: c1ccc(-c2ccc(N(c3cccc(-c4nc5c6cccc7oc8cccc(c5o4)c8c76)c3)c3ccc4c(c3)oc3ccccc34)cc2)cc1 ; c1ccc(-c2cccc(N(c3cccc(-c4nc5c6cccc7oc8cccc(c5o4)c8c76)c3)c3ccc4c(c3)oc3ccccc34)c2)cc1 ; c1ccc(N(c2cccc(-c3nc4c5cccc6oc7cccc(c4o3)c7c65)c2)c2ccc3oc4ccccc4c3c2)cc1]

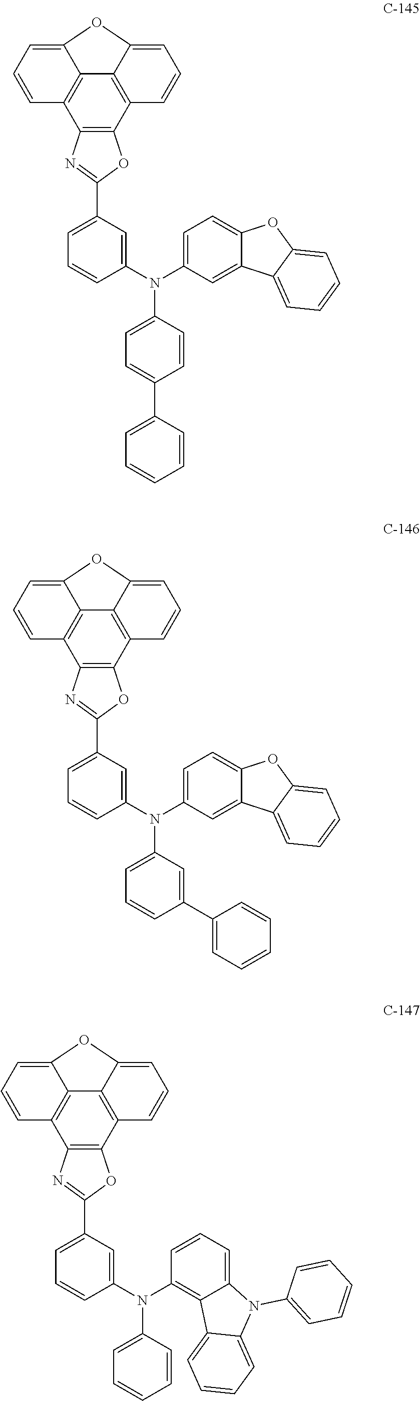 [SMILES: c1ccc(-c2ccc(N(c3cccc(-c4nc5c6cccc7oc8cccc(c5o4)c8c76)c3)c3ccc4oc5ccccc5c4c3)cc2)cc1 ; c1ccc(-c2cccc(N(c3cccc(-c4nc5c6cccc7oc8cccc(c5o4)c8c76)c3)c3ccc4oc5ccccc5c4c3)c2)cc1 ; c1ccc(N(c2cccc(-c3nc4c5cccc6oc7cccc(c4o3)c7c65)c2)c2cccc3c2c2ccccc2n3-c2ccccc2)cc1]

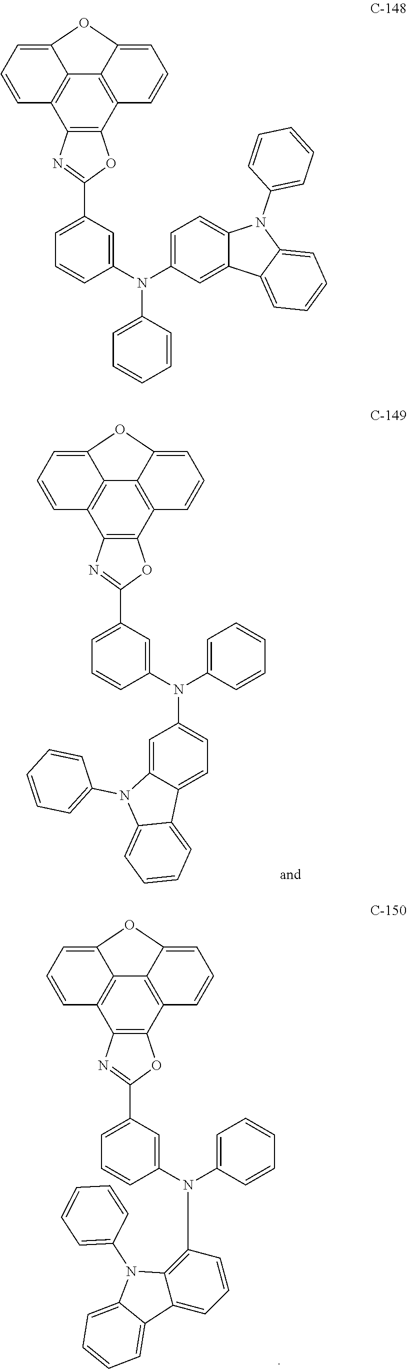

10. A plurality of host materials comprising at least one first host material comprising an organic electroluminescent compound according to claim 1 and at least one second host material different from the first host material.

11. The plurality of host materials according to claim 10, wherein second host material is comprising an organic electroluminescent compound represented by the following formula 2: 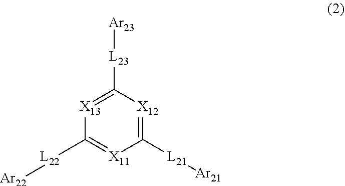
in formula 2,
X₁₁ to X₁₃ each independently represent N, CH, or CD;
L₂₁ to L₂₃ each independently represent a single bond, a substituted or unsubstituted (C6-C30)arylene, or a substituted or unsubstituted (3- to 30-membered)heteroarylene;
Ar₂₁ to Ar₂₃ each independently represent a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s).

12. The plurality of host materials according to claim 11, wherein Ar₂₁ to Ar₂₃ each independently represent a substituted or unsubstituted phenylnaphthyl, a substituted or unsubstituted naphthylphenyl, a substituted or unsubstituted phenyl, a substituted or unsubstituted biphenyl, a substituted or unsubstituted terphenyl, a substituted or unsubstituted triphenylenyl, a substituted or unsubstituted dimethylbenzofluorenyl, a substituted or unsubstituted dimethylfluorenyl, a substituted or unsubstituted phenanthrenyl, a substituted or unsubstituted benzonaphthofuranyl, a substituted or unsubstituted chrysenyl, a substituted or unsubstituted diphenylbenzofluorenyl, a substituted or unsubstituted binaphthyl, a substituted or unsubstituted dibenzofuranyl, a substituted or unsubstituted carbazolyl, a substituted or unsubstituted benzocarbazolyl, or a substituted or unsubstituted dibenzothiophenyl.

13. The plurality of host materials according to claim 11, wherein the compound represented by formula 2 is at least one selected from the following compounds.

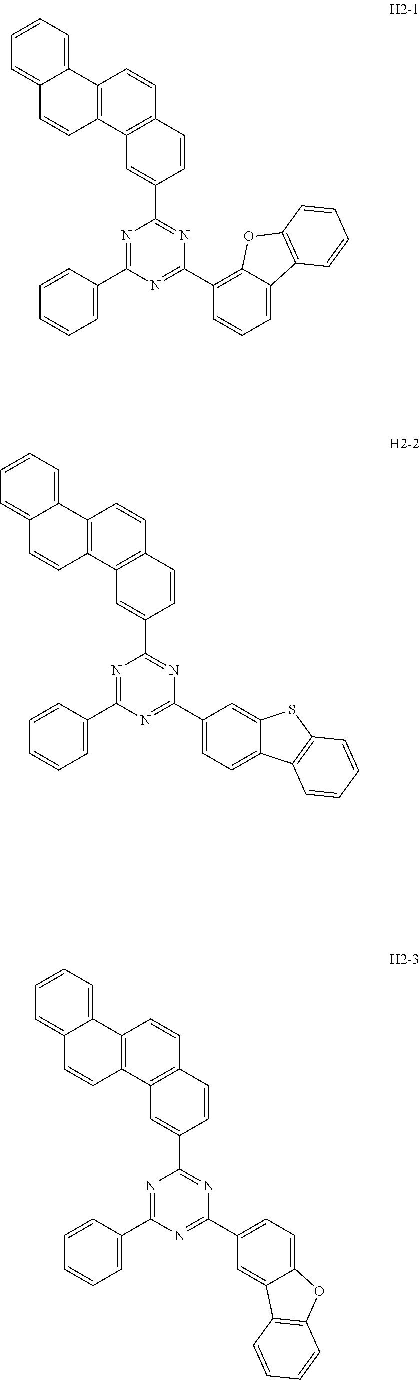 [SMILES: c1ccc(-c2nc(-c3ccc4ccc5c6ccccc6ccc5c4c3)nc(-c3cccc4c3oc3ccccc34)n2)cc1 ; c1ccc(-c2nc(-c3ccc4c(c3)sc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3ccc4oc5ccccc5c4c3)n2)cc1]

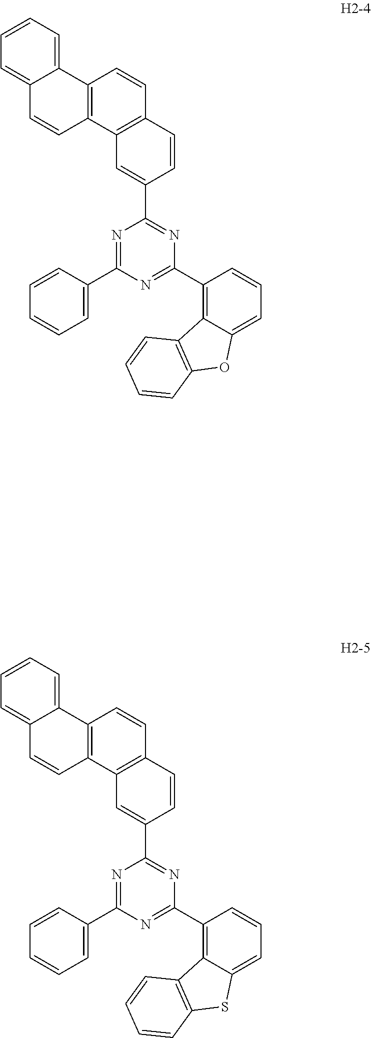 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1]

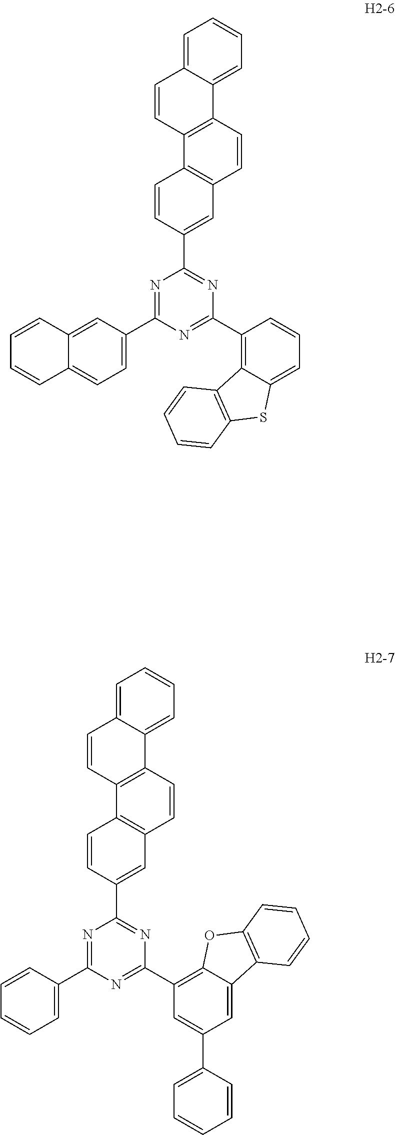 [SMILES: c1ccc2cc(-c3nc(-c4ccc5c(ccc6c7ccccc7ccc56)c4)nc(-c4cccc5sc6ccccc6c45)n3)ccc2c1 ; c1ccc(-c2cc(-c3nc(-c4ccccc4)nc(-c4ccc5c(ccc6c7ccccc7ccc56)c4)n3)c3oc4ccccc4c3c2)cc1]

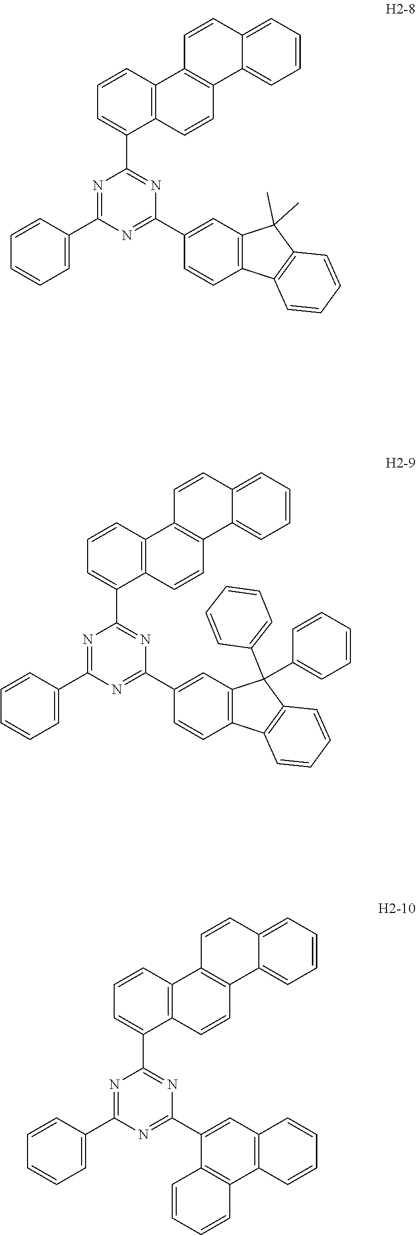 [SMILES: CC1(C)c2ccccc2-c2ccc(-c3nc(-c4ccccc4)nc(-c4cccc5c4ccc4c6ccccc6ccc54)n3)cc21 ; c1ccc(-c2nc(-c3ccc4c(c3)C(c3ccccc3)(c3ccccc3)c3ccccc3-4)nc(-c3cccc4c3ccc3c5ccccc5ccc43)n2)cc1 ; c1ccc(-c2nc(-c3cc4ccccc4c4ccccc34)nc(-c3cccc4c3ccc3c5ccccc5ccc43)n2)cc1]

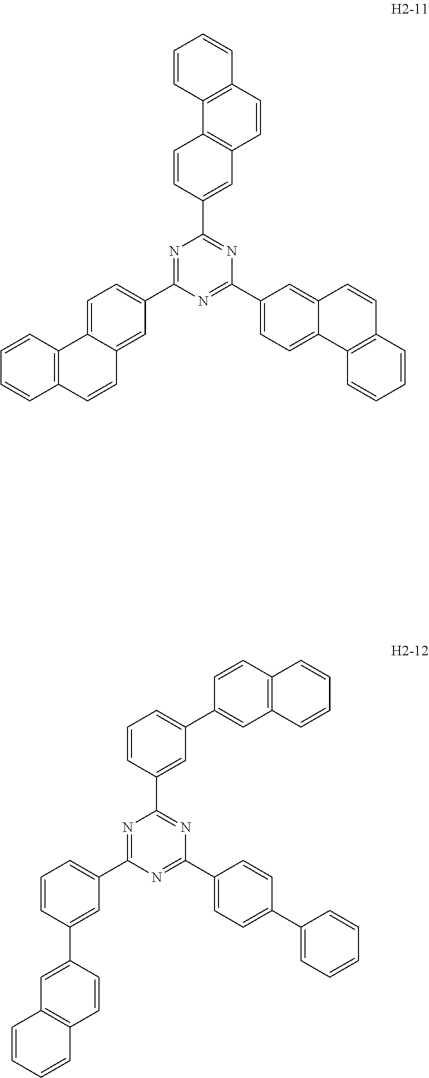 [SMILES: c1ccc2c(c1)ccc1cc(-c3nc(-c4ccc5c(ccc6ccccc65)c4)nc(-c4ccc5c(ccc6ccccc65)c4)n3)ccc12 ; c1ccc(-c2ccc(-c3nc(-c4cccc(-c5ccc6ccccc6c5)c4)nc(-c4cccc(-c5ccc6ccccc6c5)c4)n3)cc2)cc1]

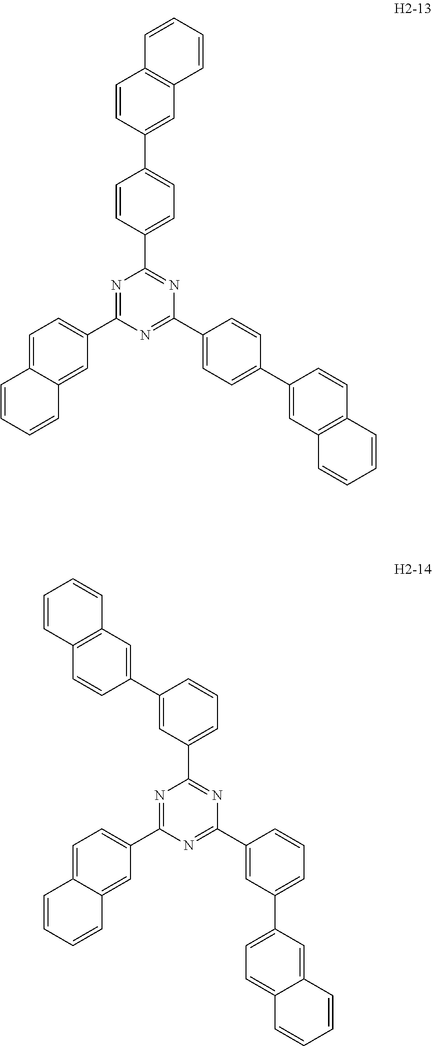 [SMILES: c1ccc2cc(-c3ccc(-c4nc(-c5ccc(-c6ccc7ccccc7c6)cc5)nc(-c5ccc6ccccc6c5)n4)cc3)ccc2c1 ; c1cc(-c2ccc3ccccc3c2)cc(-c2nc(-c3cccc(-c4ccc5ccccc5c4)c3)nc(-c3ccc4ccccc4c3)n2)c1]

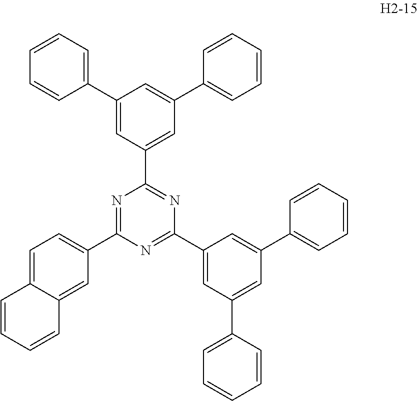 [SMILES: c1ccc(-c2cc(-c3ccccc3)cc(-c3nc(-c4cc(-c5ccccc5)cc(-c5ccccc5)c4)nc(-c4ccc5ccccc5c4)n3)c2)cc1]

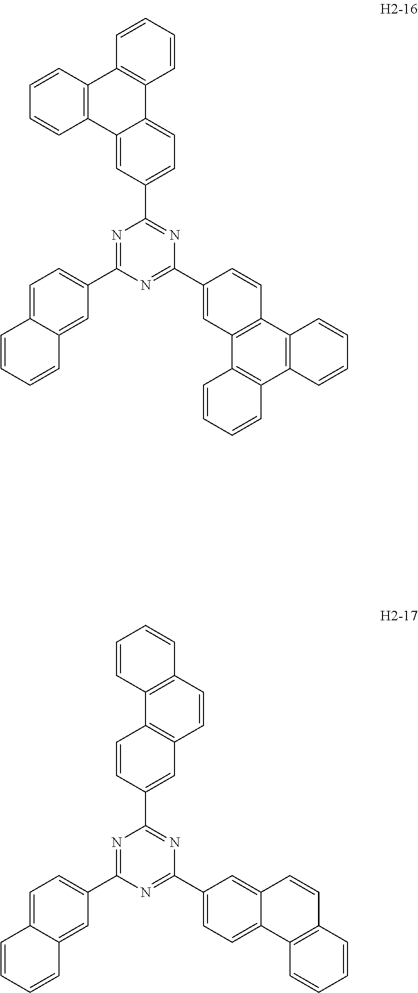 [SMILES: c1ccc2cc(-c3nc(-c4ccc5c6ccccc6c6ccccc6c5c4)nc(-c4ccc5c6ccccc6c6ccccc6c5c4)n3)ccc2c1 ; c1ccc2cc(-c3nc(-c4ccc5c(ccc6ccccc65)c4)nc(-c4ccc5c(ccc6ccccc65)c4)n3)ccc2c1]

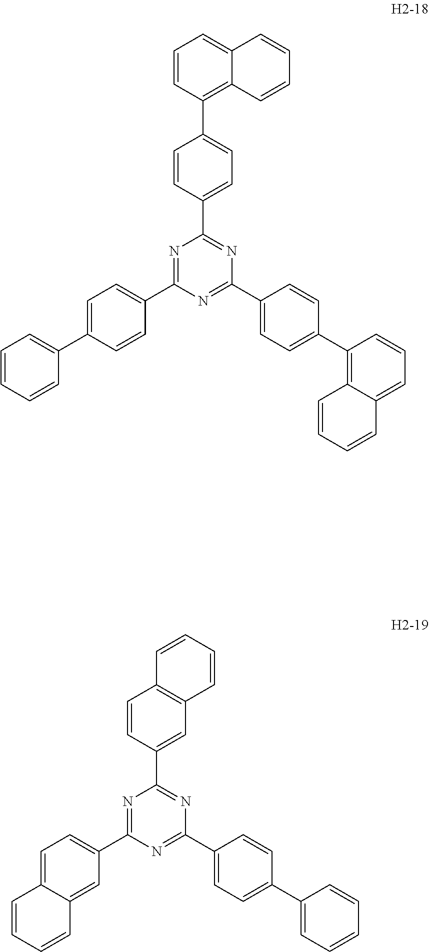 [SMILES: c1ccc(-c2ccc(-c3nc(-c4ccc(-c5cccc6ccccc56)cc4)nc(-c4ccc(-c5cccc6ccccc56)cc4)n3)cc2)cc1 ; c1ccc(-c2ccc(-c3nc(-c4ccc5ccccc5c4)nc(-c4ccc5ccccc5c4)n3)cc2)cc1]

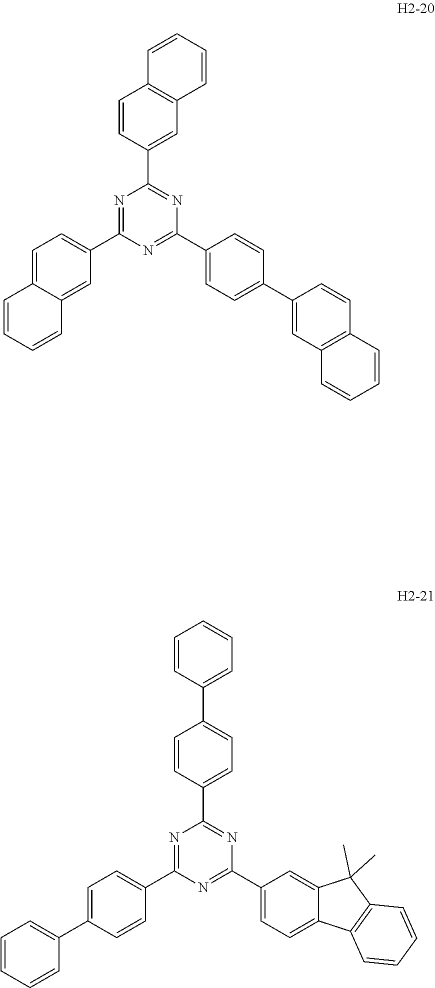 [SMILES: c1ccc2cc(-c3ccc(-c4nc(-c5ccc6ccccc6c5)nc(-c5ccc6ccccc6c5)n4)cc3)ccc2c1 ; CC1(C)c2ccccc2-c2ccc(-c3nc(-c4ccc(-c5ccccc5)cc4)nc(-c4ccc(-c5ccccc5)cc4)n3)cc21]

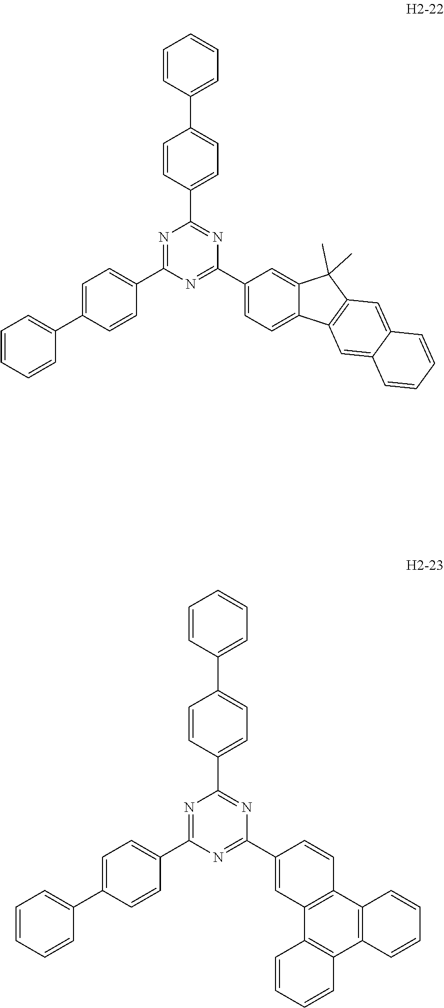 [SMILES: CC1(C)c2cc(-c3nc(-c4ccc(-c5ccccc5)cc4)nc(-c4ccc(-c5ccccc5)cc4)n3)ccc2-c2cc3ccccc3cc21 ; c1ccc(-c2ccc(-c3nc(-c4ccc(-c5ccccc5)cc4)nc(-c4ccc5c6ccccc6c6ccccc6c5c4)n3)cc2)cc1]

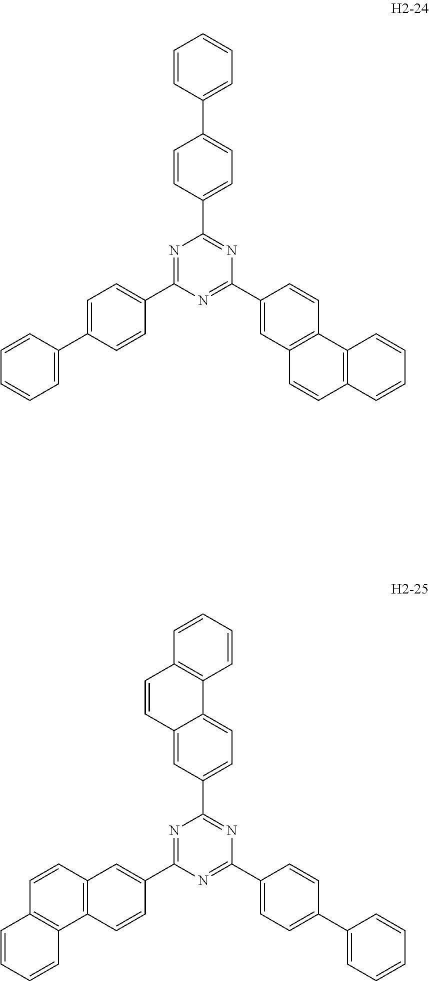 [SMILES: c1ccc(-c2ccc(-c3nc(-c4ccc(-c5ccccc5)cc4)nc(-c4ccc5c(ccc6ccccc65)c4)n3)cc2)cc1 ; c1ccc(-c2ccc(-c3nc(-c4ccc5c(ccc6ccccc65)c4)nc(-c4ccc5c(ccc6ccccc65)c4)n3)cc2)cc1]

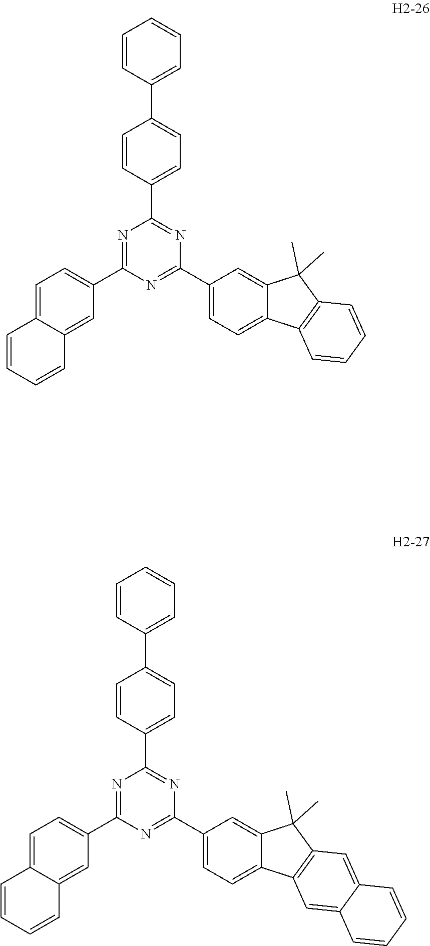 [SMILES: CC1(C)c2ccccc2-c2ccc(-c3nc(-c4ccc(-c5ccccc5)cc4)nc(-c4ccc5ccccc5c4)n3)cc21 ; CC1(C)c2cc(-c3nc(-c4ccc(-c5ccccc5)cc4)nc(-c4ccc5ccccc5c4)n3)ccc2-c2cc3ccccc3cc21]

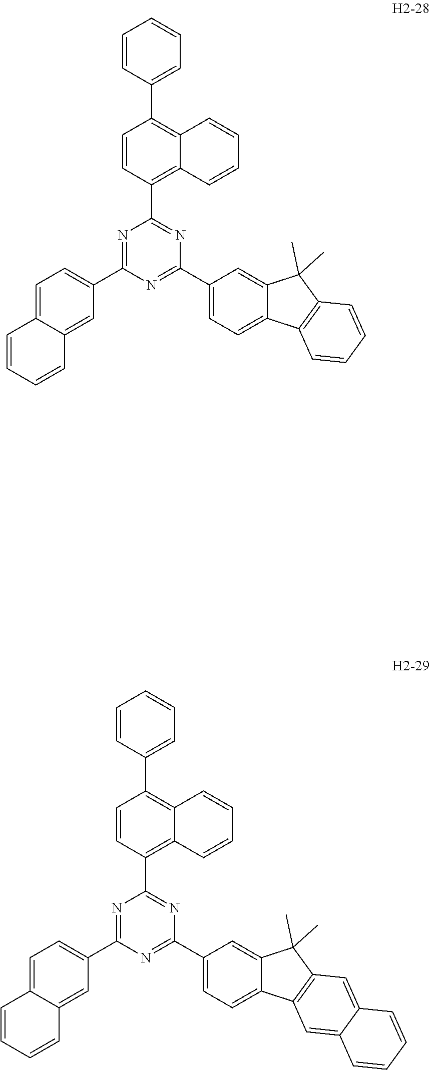 [SMILES: CC1(C)c2ccccc2-c2ccc(-c3nc(-c4ccc5ccccc5c4)nc(-c4ccc(-c5ccccc5)c5ccccc45)n3)cc21 ; CC1(C)c2cc(-c3nc(-c4ccc5ccccc5c4)nc(-c4ccc(-c5ccccc5)c5ccccc45)n3)ccc2-c2cc3ccccc3cc21]

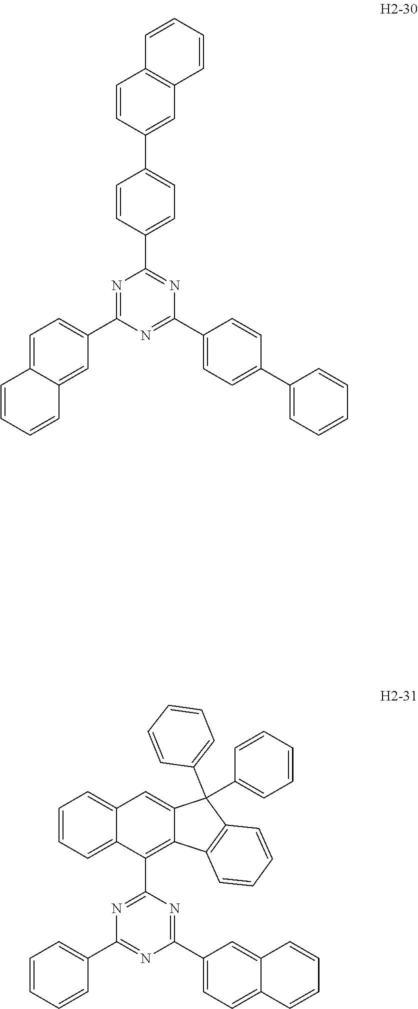 [SMILES: c1ccc(-c2ccc(-c3nc(-c4ccc(-c5ccc6ccccc6c5)cc4)nc(-c4ccc5ccccc5c4)n3)cc2)cc1 ; c1ccc(-c2nc(-c3ccc4ccccc4c3)nc(-c3c4c(cc5ccccc35)C(c3ccccc3)(c3ccccc3)c3ccccc3-4)n2)cc1]

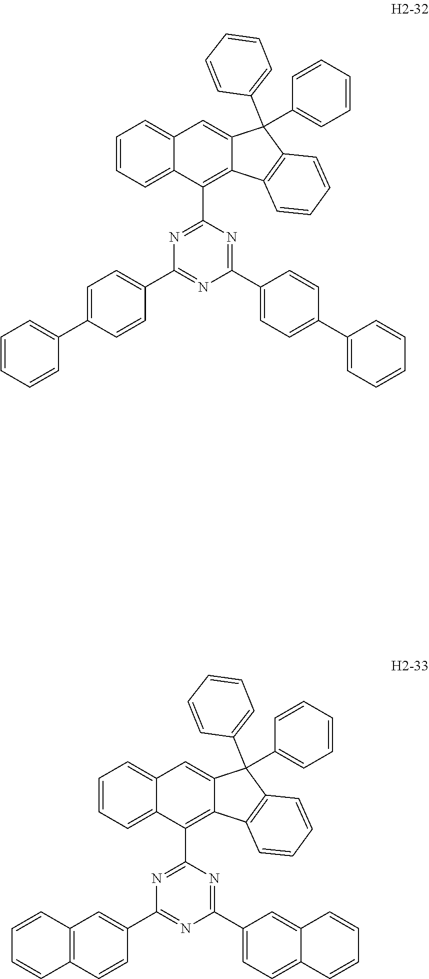 [SMILES: c1ccc(-c2ccc(-c3nc(-c4ccc(-c5ccccc5)cc4)nc(-c4c5c(cc6ccccc46)C(c4ccccc4)(c4ccccc4)c4ccccc4-5)n3)cc2)cc1 ; c1ccc(C2(c3ccccc3)c3ccccc3-c3c2cc2ccccc2c3-c2nc(-c3ccc4ccccc4c3)nc(-c3ccc4ccccc4c3)n2)cc1]

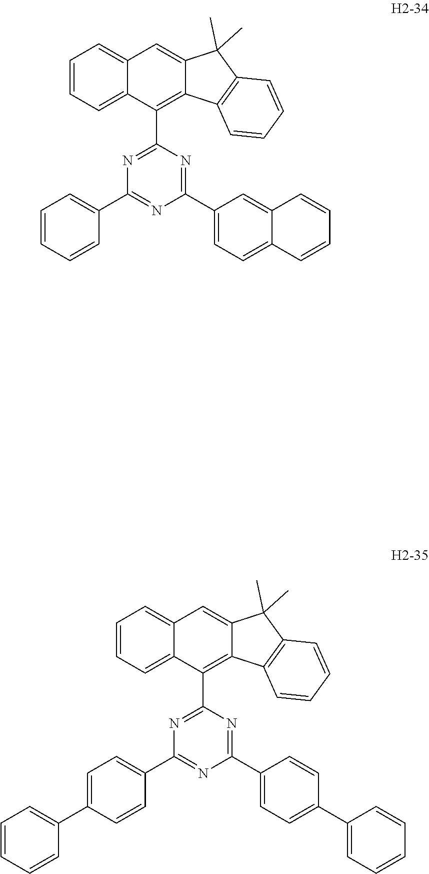 [SMILES: CC1(C)c2ccccc2-c2c1cc1ccccc1c2-c1nc(-c2ccccc2)nc(-c2ccc3ccccc3c2)n1 ; CC1(C)c2ccccc2-c2c1cc1ccccc1c2-c1nc(-c2ccc(-c3ccccc3)cc2)nc(-c2ccc(-c3ccccc3)cc2)n1]

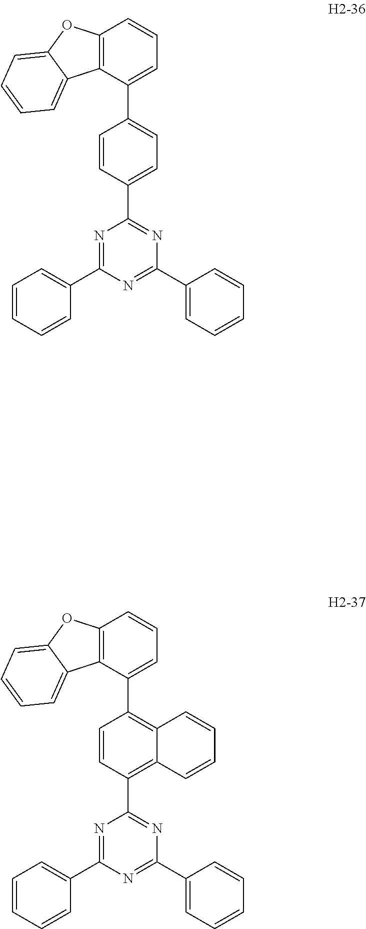 [SMILES: c1ccc(-c2nc(-c3ccccc3)nc(-c3ccc(-c4cccc5oc6ccccc6c45)cc3)n2)cc1 ; c1ccc(-c2nc(-c3ccccc3)nc(-c3ccc(-c4cccc5oc6ccccc6c45)c4ccccc34)n2)cc1]

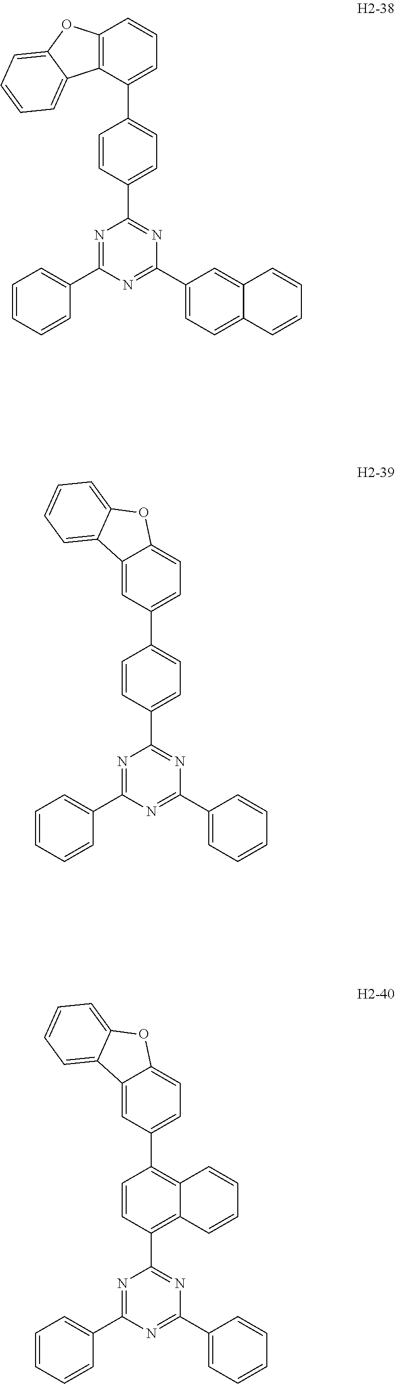 [SMILES: c1ccc(-c2nc(-c3ccc(-c4cccc5oc6ccccc6c45)cc3)nc(-c3ccc4ccccc4c3)n2)cc1 ; c1ccc(-c2nc(-c3ccccc3)nc(-c3ccc(-c4ccc5oc6ccccc6c5c4)cc3)n2)cc1 ; c1ccc(-c2nc(-c3ccccc3)nc(-c3ccc(-c4ccc5oc6ccccc6c5c4)c4ccccc34)n2)cc1]

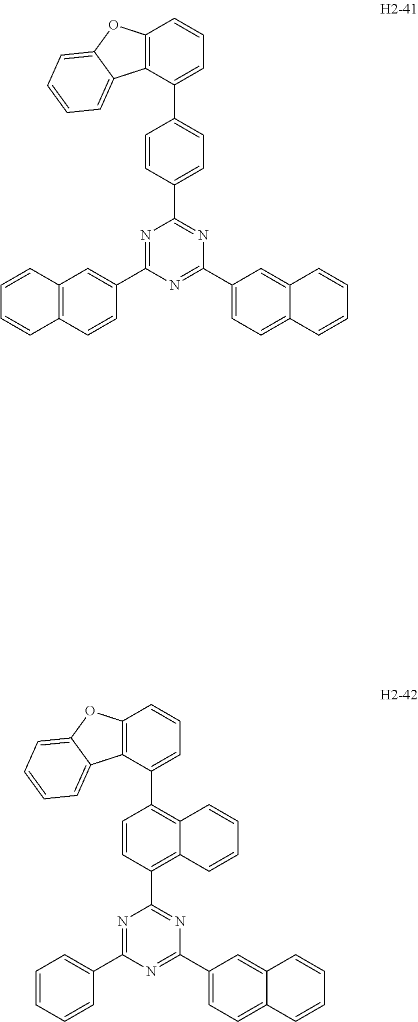 [SMILES: c1ccc2cc(-c3nc(-c4ccc(-c5cccc6oc7ccccc7c56)cc4)nc(-c4ccc5ccccc5c4)n3)ccc2c1 ; c1ccc(-c2nc(-c3ccc4ccccc4c3)nc(-c3ccc(-c4cccc5oc6ccccc6c45)c4ccccc34)n2)cc1]

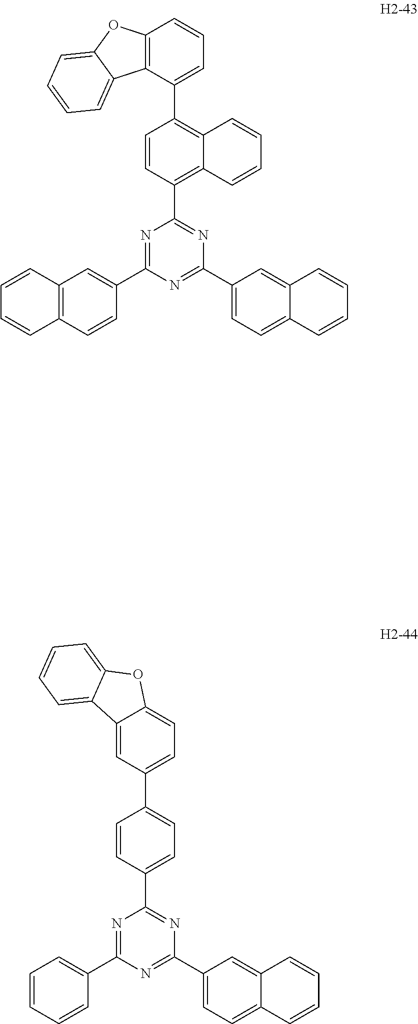 [SMILES: c1ccc2cc(-c3nc(-c4ccc5ccccc5c4)nc(-c4ccc(-c5cccc6oc7ccccc7c56)c5ccccc45)n3)ccc2c1 ; c1ccc(-c2nc(-c3ccc(-c4ccc5oc6ccccc6c5c4)cc3)nc(-c3ccc4ccccc4c3)n2)cc1]

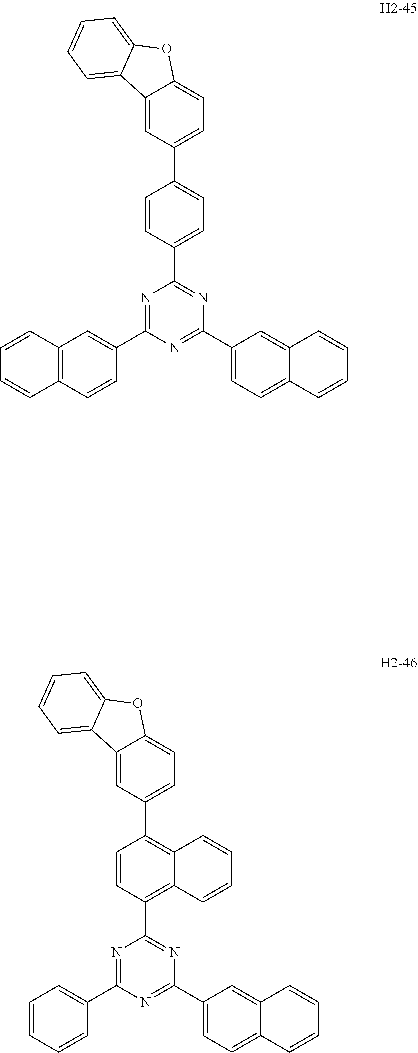 [SMILES: c1ccc2cc(-c3nc(-c4ccc(-c5ccc6oc7ccccc7c6c5)cc4)nc(-c4ccc5ccccc5c4)n3)ccc2c1 ; c1ccc(-c2nc(-c3ccc4ccccc4c3)nc(-c3ccc(-c4ccc5oc6ccccc6c5c4)c4ccccc34)n2)cc1]

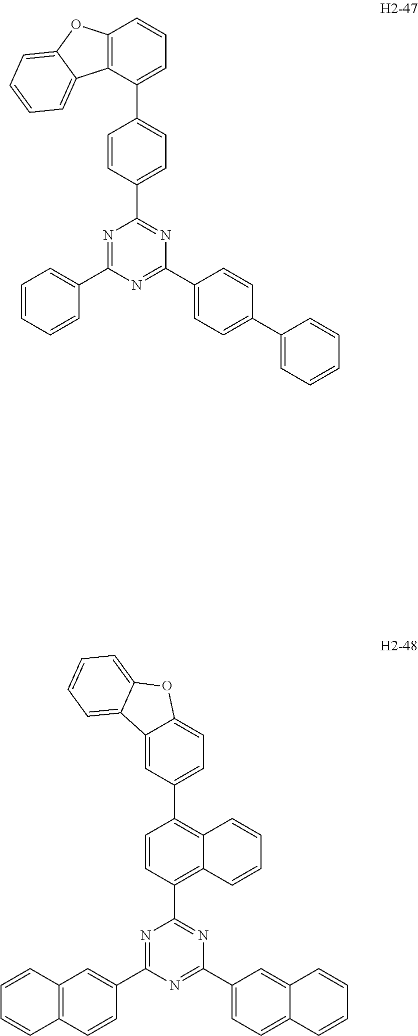 [SMILES: c1ccc(-c2ccc(-c3nc(-c4ccccc4)nc(-c4ccc(-c5cccc6oc7ccccc7c56)cc4)n3)cc2)cc1 ; c1ccc2cc(-c3nc(-c4ccc5ccccc5c4)nc(-c4ccc(-c5ccc6oc7ccccc7c6c5)c5ccccc45)n3)ccc2c1]

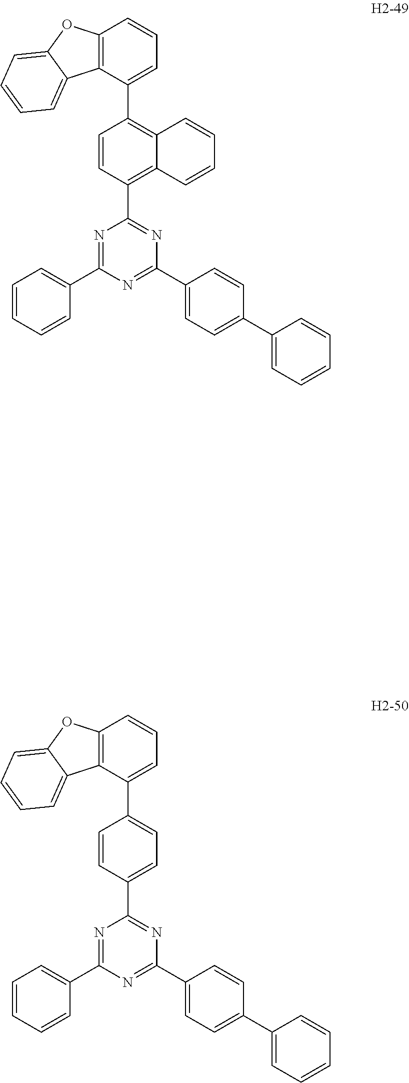 [SMILES: c1ccc(-c2ccc(-c3nc(-c4ccccc4)nc(-c4ccc(-c5cccc6oc7ccccc7c56)c5ccccc45)n3)cc2)cc1 ; c1ccc(-c2ccc(-c3nc(-c4ccccc4)nc(-c4ccc(-c5cccc6oc7ccccc7c56)cc4)n3)cc2)cc1]

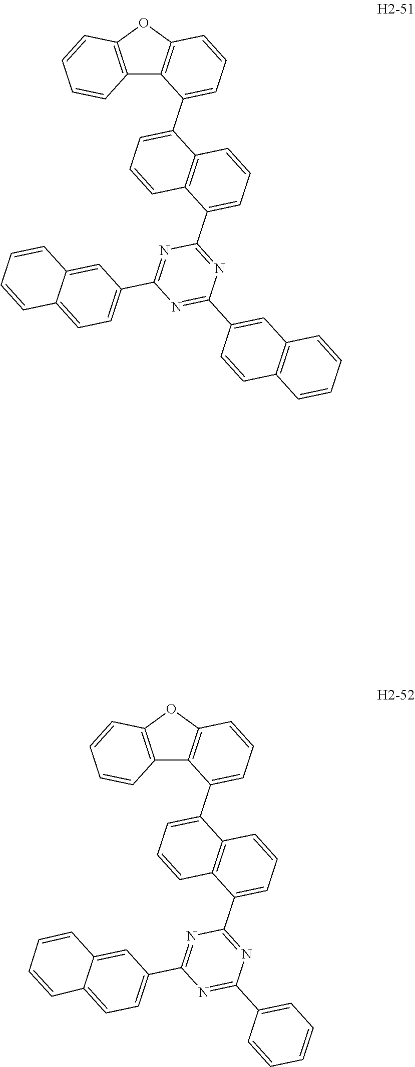 [SMILES: c1ccc2cc(-c3nc(-c4ccc5ccccc5c4)nc(-c4cccc5c(-c6cccc7oc8ccccc8c67)cccc45)n3)ccc2c1 ; c1ccc(-c2nc(-c3ccc4ccccc4c3)nc(-c3cccc4c(-c5cccc6oc7ccccc7c56)cccc34)n2)cc1]

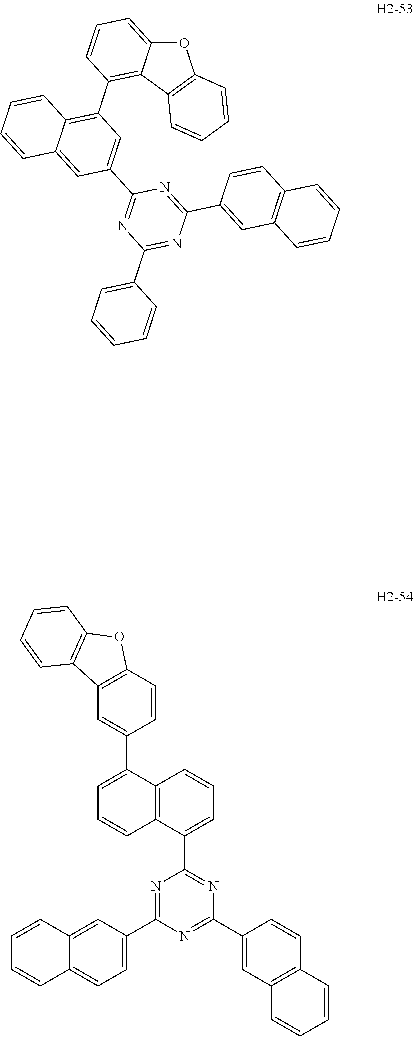 [SMILES: c1ccc(-c2nc(-c3ccc4ccccc4c3)nc(-c3cc(-c4cccc5oc6ccccc6c45)c4ccccc4c3)n2)cc1 ; c1ccc2cc(-c3nc(-c4ccc5ccccc5c4)nc(-c4cccc5c(-c6ccc7oc8ccccc8c7c6)cccc45)n3)ccc2c1]

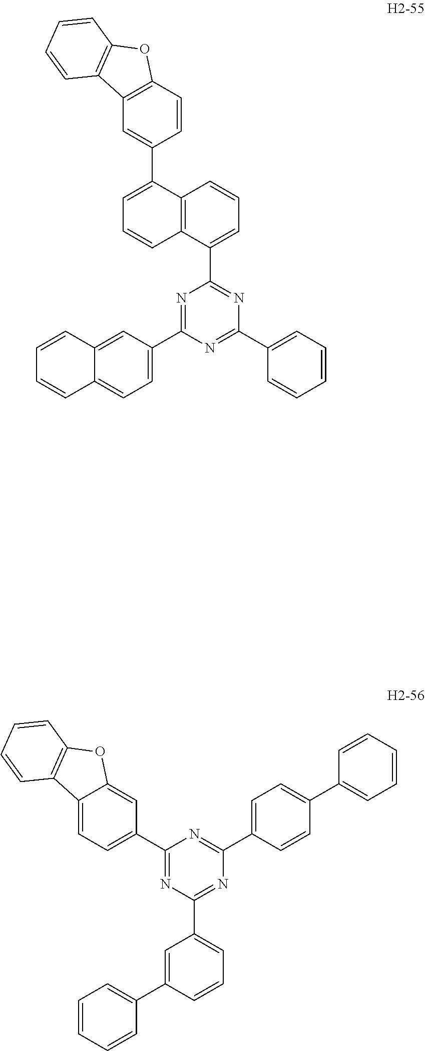 [SMILES: c1ccc(-c2nc(-c3ccc4ccccc4c3)nc(-c3cccc4c(-c5ccc6oc7ccccc7c6c5)cccc34)n2)cc1 ; c1ccc(-c2ccc(-c3nc(-c4cccc(-c5ccccc5)c4)nc(-c4ccc5c(c4)oc4ccccc45)n3)cc2)cc1]

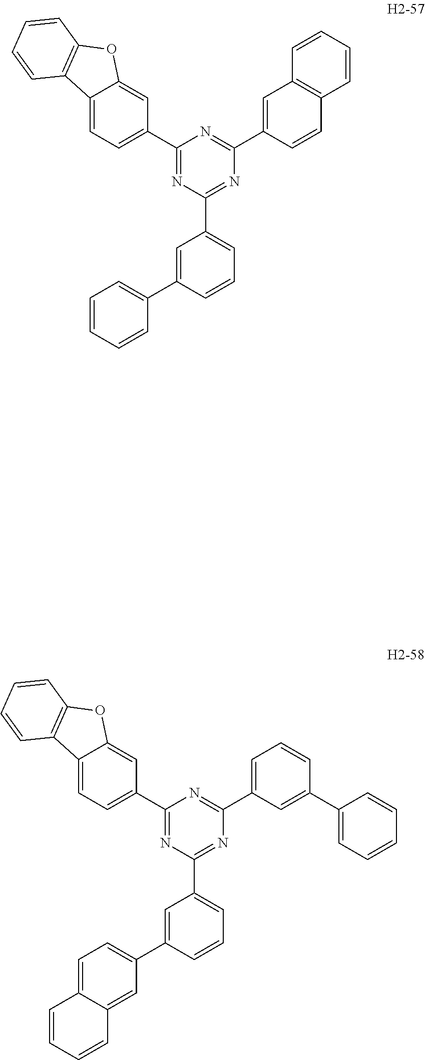 [SMILES: c1ccc(-c2cccc(-c3nc(-c4ccc5ccccc5c4)nc(-c4ccc5c(c4)oc4ccccc45)n3)c2)cc1 ; c1ccc(-c2cccc(-c3nc(-c4cccc(-c5ccc6ccccc6c5)c4)nc(-c4ccc5c(c4)oc4ccccc45)n3)c2)cc1]

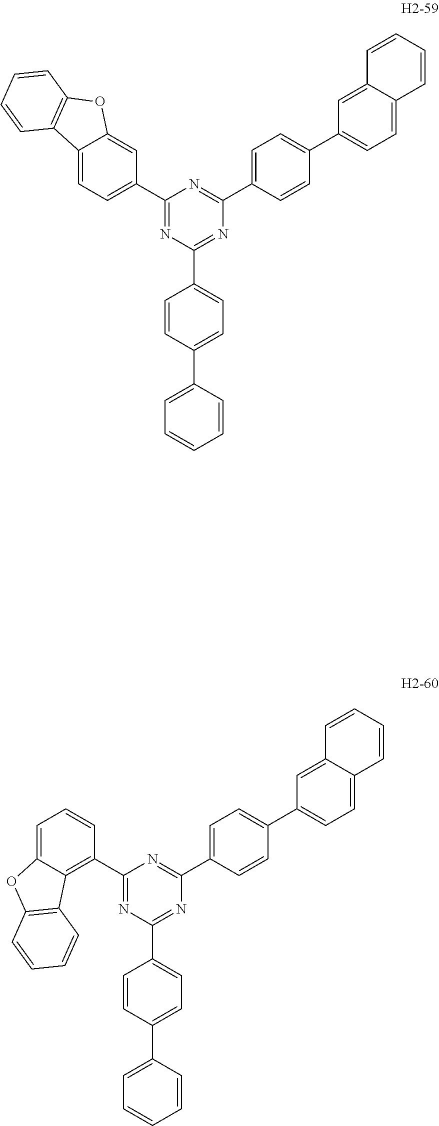 [SMILES: c1ccc(-c2ccc(-c3nc(-c4ccc(-c5ccc6ccccc6c5)cc4)nc(-c4ccc5c(c4)oc4ccccc45)n3)cc2)cc1 ; c1ccc(-c2ccc(-c3nc(-c4ccc(-c5ccc6ccccc6c5)cc4)nc(-c4cccc5oc6ccccc6c45)n3)cc2)cc1]

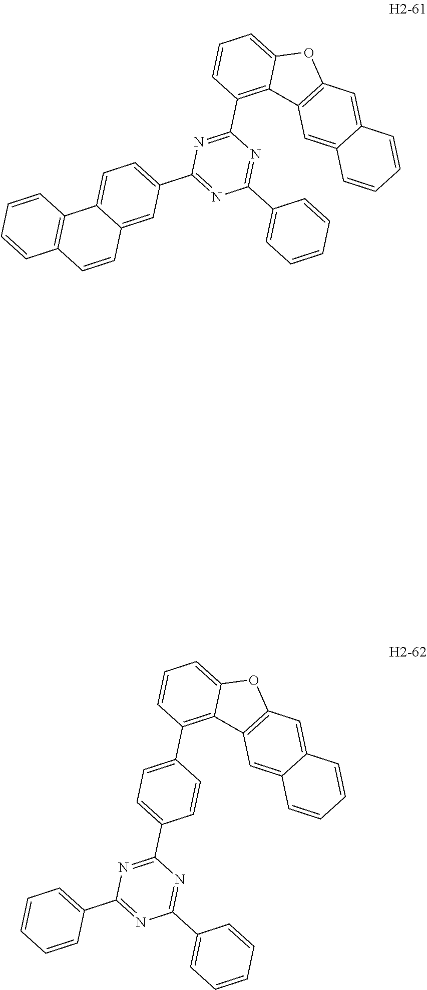 [SMILES: c1ccc(-c2nc(-c3ccc4c(ccc5ccccc54)c3)nc(-c3cccc4oc5cc6ccccc6cc5c34)n2)cc1 ; c1ccc(-c2nc(-c3ccccc3)nc(-c3ccc(-c4cccc5oc6cc7ccccc7cc6c45)cc3)n2)cc1]

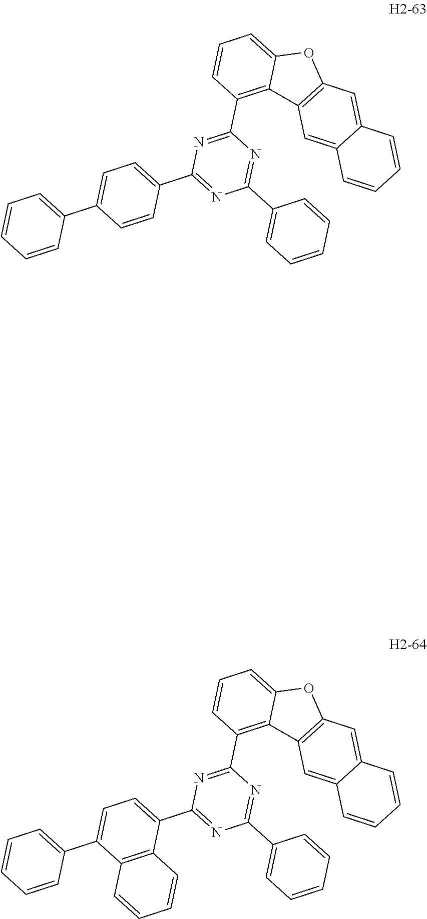 [SMILES: c1ccc(-c2ccc(-c3nc(-c4ccccc4)nc(-c4cccc5oc6cc7ccccc7cc6c45)n3)cc2)cc1 ; c1ccc(-c2nc(-c3ccc(-c4ccccc4)c4ccccc34)nc(-c3cccc4oc5cc6ccccc6cc5c34)n2)cc1]

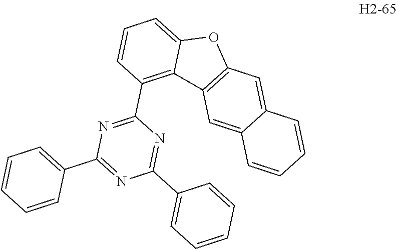 [SMILES: c1ccc(-c2nc(-c3ccccc3)nc(-c3cccc4oc5cc6ccccc6cc5c34)n2)cc1]

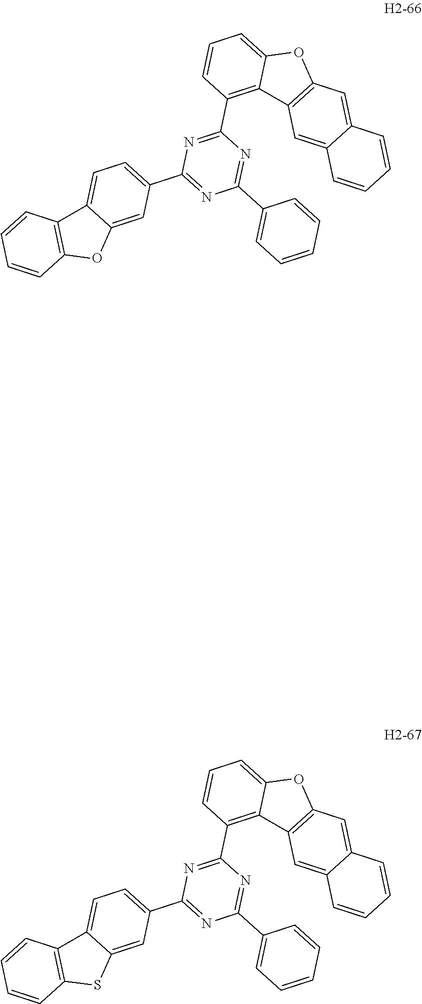 [SMILES: c1ccc(-c2nc(-c3ccc4c(c3)oc3ccccc34)nc(-c3cccc4oc5cc6ccccc6cc5c34)n2)cc1 ; c1ccc(-c2nc(-c3ccc4c(c3)sc3ccccc34)nc(-c3cccc4oc5cc6ccccc6cc5c34)n2)cc1]

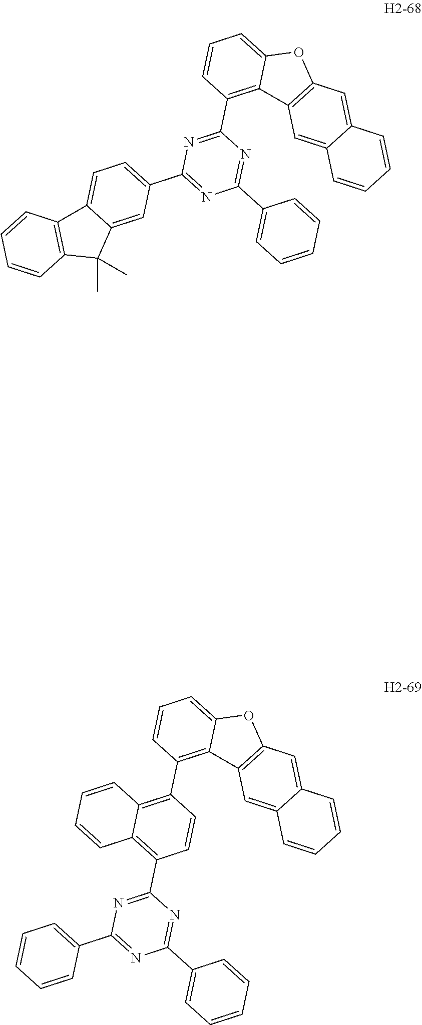 [SMILES: CC1(C)c2ccccc2-c2ccc(-c3nc(-c4ccccc4)nc(-c4cccc5oc6cc7ccccc7cc6c45)n3)cc21 ; c1ccc(-c2nc(-c3ccccc3)nc(-c3ccc(-c4cccc5oc6cc7ccccc7cc6c45)c4ccccc34)n2)cc1]

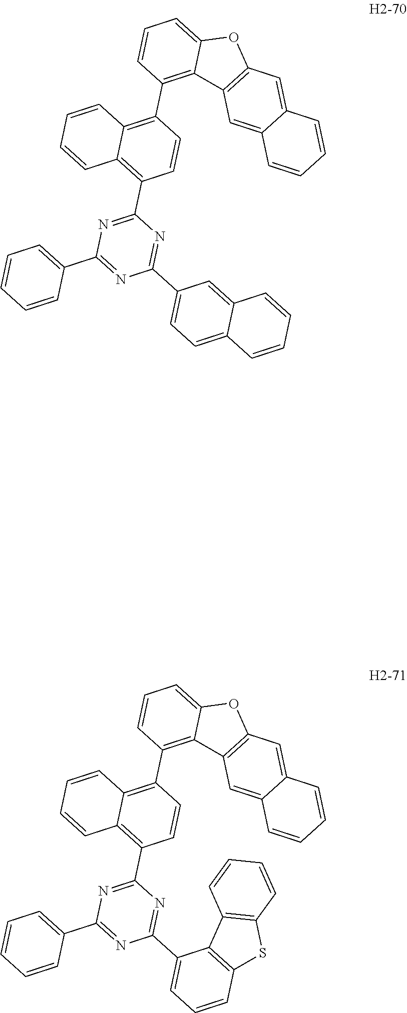 [SMILES: c1ccc(-c2nc(-c3ccc4ccccc4c3)nc(-c3ccc(-c4cccc5oc6cc7ccccc7cc6c45)c4ccccc34)n2)cc1 ; c1ccc(-c2nc(-c3ccc(-c4cccc5oc6cc7ccccc7cc6c45)c4ccccc34)nc(-c3cccc4sc5ccccc5c34)n2)cc1]

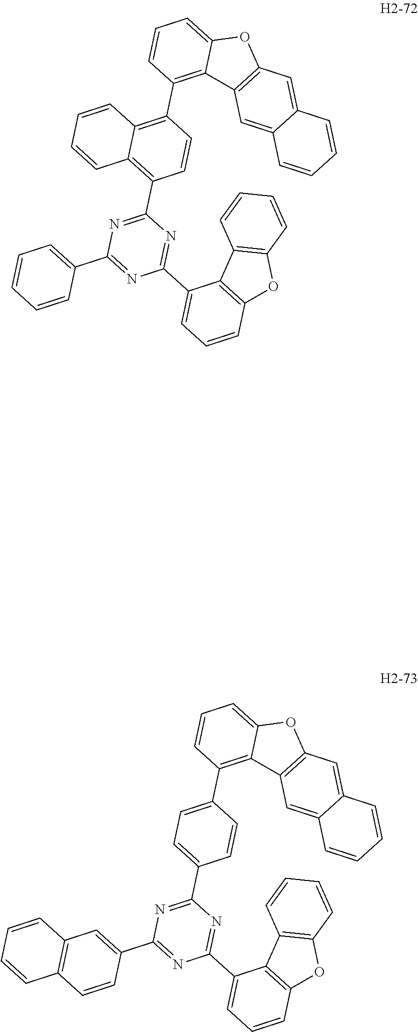 [SMILES: c1ccc(-c2nc(-c3ccc(-c4cccc5oc6cc7ccccc7cc6c45)c4ccccc34)nc(-c3cccc4oc5ccccc5c34)n2)cc1 ; c1ccc2cc(-c3nc(-c4ccc(-c5cccc6oc7cc8ccccc8cc7c56)cc4)nc(-c4cccc5oc6ccccc6c45)n3)ccc2c1]

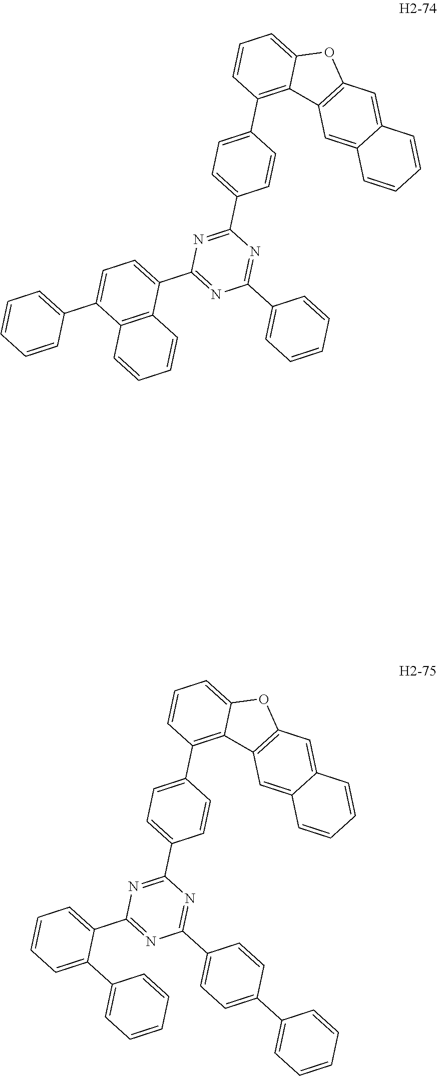 [SMILES: c1ccc(-c2nc(-c3ccc(-c4cccc5oc6cc7ccccc7cc6c45)cc3)nc(-c3ccc(-c4ccccc4)c4ccccc34)n2)cc1 ; c1ccc(-c2ccc(-c3nc(-c4ccc(-c5cccc6oc7cc8ccccc8cc7c56)cc4)nc(-c4ccccc4-c4ccccc4)n3)cc2)cc1]

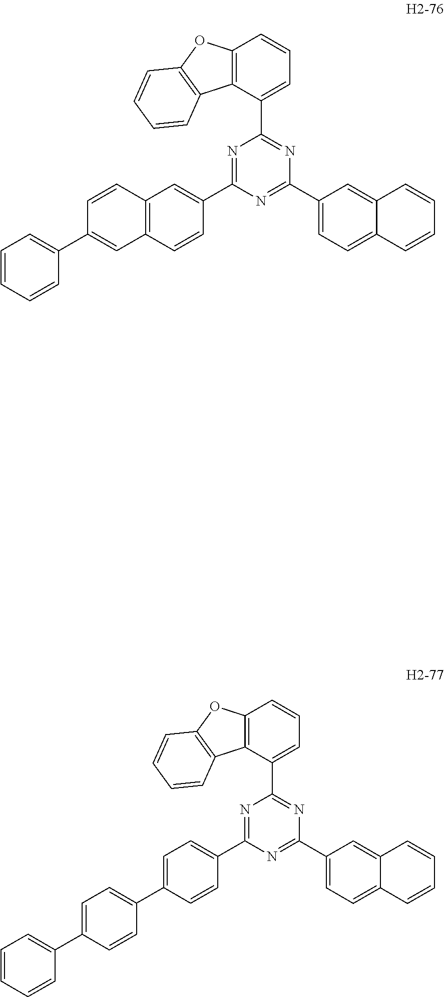 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccc6ccccc6c5)nc(-c5cccc6oc7ccccc7c56)n4)cc3)cc2)cc1]

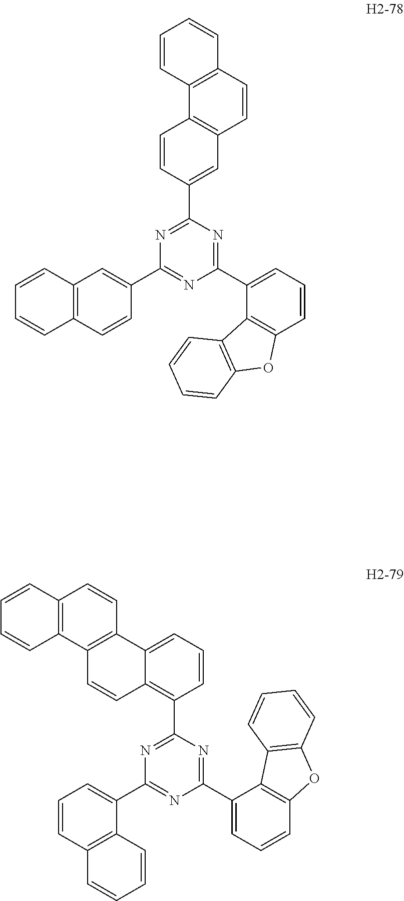 [SMILES: c1ccc2cc(-c3nc(-c4ccc5c(ccc6ccccc65)c4)nc(-c4cccc5oc6ccccc6c45)n3)ccc2c1 ; c1ccc2c(-c3nc(-c4cccc5c4ccc4c6ccccc6ccc54)nc(-c4cccc5oc6ccccc6c45)n3)cccc2c1]

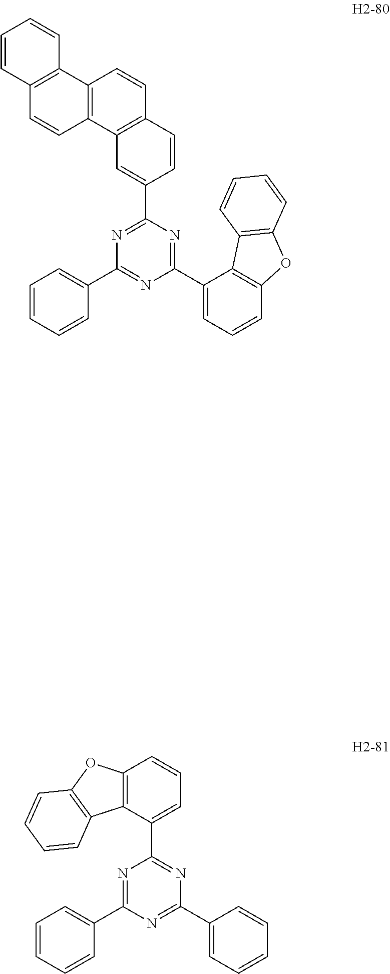 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccccc3)nc(-c3cccc4oc5ccccc5c34)n2)cc1]

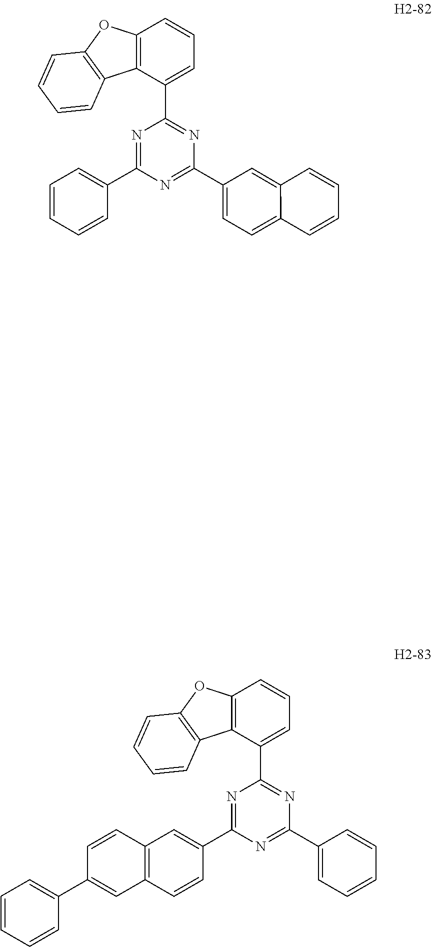 [SMILES: c1ccc(-c2nc(-c3ccc4ccccc4c3)nc(-c3cccc4oc5ccccc5c34)n2)cc1 ; c1ccc(-c2ccc3cc(-c4nc(-c5ccccc5)nc(-c5cccc6oc7ccccc7c56)n4)ccc3c2)cc1]

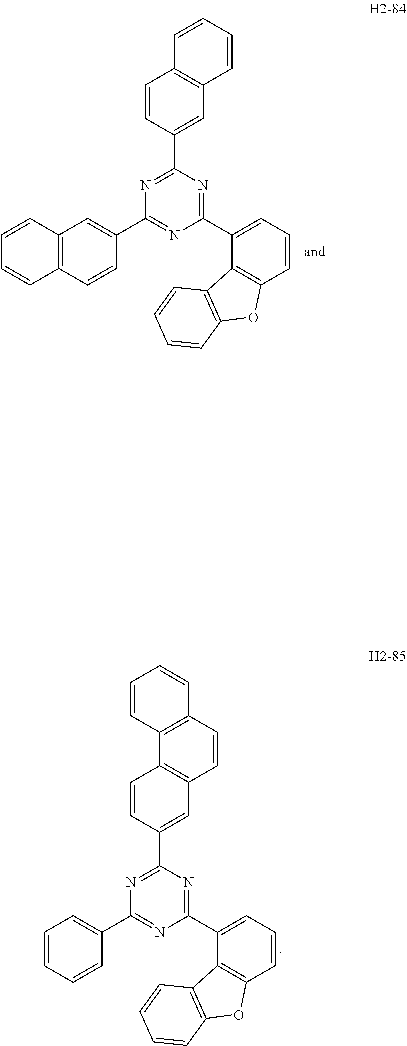 [SMILES: c1ccc2cc(-c3nc(-c4ccc5ccccc5c4)nc(-c4cccc5oc6ccccc6c45)n3)ccc2c1 ; c1ccc(-c2nc(-c3ccc4c(ccc5ccccc54)c3)nc(-c3cccc4oc5ccccc5c34)n2)cc1]

14. An organic electroluminescent device comprising the organic electroluminescent compound according to claim 1.

15. An organic electroluminescent device comprising an anode; a cathode; and at least one light-emitting layer between the anode and the cathode, wherein the at least one light-emitting layer comprises the plurality of host materials according to claim 10.
